SEQUENCE LISTING (44 sequences)

SEQ ID NO: 1 - protein, 5 aa, synthetic construct
DYYMS

SEQ ID NO: 2 - protein, 17 aa, synthetic construct
YISSSGSTIYYADSVKG

SEQ ID NO: 3 - protein, 10 aa, synthetic construct
DFYDSKIFDY

SEQ ID NO: 4 - protein, 11 aa, synthetic construct
RASQSISSYLN

SEQ ID NO: 5 - protein, 7 aa, synthetic construct
AASSLQS

SEQ ID NO: 6 - protein, 9 aa, synthetic construct
MEGLKTPFT

SEQ ID NO: 7 - protein, 119 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDFYDSKIFDYWGQGTLVTVSS

SEQ ID NO: 8 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDVGVYYCMEGLKTPFTFGPGTKLEIKR

SEQ ID NO: 9 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 10 - protein, 8 aa, synthetic construct
EPKSCDKT

SEQ ID NO: 11 - protein, 225 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDFYDSKIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 12 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDVGVYYCMEGLKTPFTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 449 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDFYDSKIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 214 aa, synthetic construct; identical to SEQ ID NO: 13

SEQ ID NO: 16 - DNA, 15 bp, synthetic construct
  source 1..15: mol_type = other DNA
gattactatatgagc

SEQ ID NO: 17 - DNA, 51 bp, synthetic construct
  source 1..51: mol_type = other DNA
tacatctcaagttccggctctacaatctactatgcagattccgtgaaaggg

SEQ ID NO: 18 - DNA, 30 bp, synthetic construct
  source 1..30: mol_type = other DNA
gatttctatgatagcaagatttttgactac

SEQ ID NO: 19 - DNA, 33 bp, synthetic construct
  source 1..33: mol_type = other DNA
cgtgcatcccaaagcatttcgagttacttgaac

SEQ ID NO: 20 - DNA, 21 bp, synthetic construct
  source 1..21: mol_type = other DNA
gctgccagtagtcttcaatct

SEQ ID NO: 21 - DNA, 27 bp, synthetic construct
  source 1..27: mol_type = other DNA
atggaaggtttaaaaactccattcact

SEQ ID NO: 22 - DNA, 357 bp, synthetic construct
  source 1..357: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggctgtgtattactgtgcgagagatttctatgatagcaagatttttgactactggggccagggaaccctggtcaccgtctcctca

SEQ ID NO: 23 - DNA, 324 bp, synthetic construct
  source 1..324: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaggatgtcggagtttattactgcatggaaggtttaaaaactccattcactttcggccctgggaccaaactggagatcaaacgt

SEQ ID NO: 24 - DNA, 294 bp, synthetic construct
  source 1..294: mol_type = other DNA
gcgtcgaccaaagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctcttcatctttagggacccaaacctatatctgcaacgtcaatcataagccttcaaataccaaggttgacaaaaaggtg

SEQ ID NO: 25 - DNA, 24 bp, synthetic construct
  source 1..24: mol_type = other DNA
gagcccaaatcttgtgacaaaact

SEQ ID NO: 26 - DNA, 675 bp, synthetic construct
  source 1..675: mol_type = other DNA
caagttcaattagtggagagcggcggcggattggttaaaccggggggcagtctgcgtttaagctgcgcagcttccgggtttactttttcggattactatatgagctggattcgtcaggctcccggtaaagggttggaatgggttagctacatctcaagttccggctctacaatctactatgcagattccgtgaaagggcgcttcaccatctctcgtgacaatgcaaagaacagtttatacttacaaatgaactcgctgcgtgcggaagatacggctgtgtattactgtgcgagagatttctatgatagcaagatttttgactactggggccagggaaccctggtcaccgtctcctcagcgtcgaccaaagggcccagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctcttcatctttagggacccaaacctatatctgcaacgtcaatcataagccttcaaataccaaggttgacaaaaaggtggagcccaaatcttgtgacaaaact

SEQ ID NO: 27 - DNA, 318 bp, synthetic construct
  source 1..318: mol_type = other DNA
acggtggctgctccgtcggtgtttatctttccaccgagtgacgaacaactgaagtcggggactgccagcgttgtttgccttctgaacaatttttacccccgcgaggcgaaggttcagtggaaagtcgataatgcgttacagagcgggaatagccaggaaagtgttactgaacaggatagtaaagacagcacttacagcttatcgtctacacttactctgagtaaagcggattatgaaaagcataaggtgtacgcttgcgaggttacacatcaagggttatcttcgccagtcacgaagagctttaatcgcggcgagtgc

SEQ ID NO: 28 - DNA, 642 bp, synthetic construct
  source 1..642: mol_type = other DNA
gatattcaaatgacccagtctccttcttccttgagcgcgtctgtgggagaccgtgtaactatcacgtgtcgtgcatcccaaagcatttcgagttacttgaactggtatcaacaaaagcctggtaaagcgcccaaactgttgatctatgctgccagtagtcttcaatctggtgtgccctcgcgcttctcaggttcggggtcaggaacggactttaccctgaccattagctctctgcaaccggaggatgtcggagtttattactgcatggaaggtttaaaaactccattcactttcggccctgggaccaaactggagatcaaacgtacggtggctgctccgtcggtgtttatctttccaccgagtgacgaacaactgaagtcggggactgccagcgttgtttgccttctgaacaatttttacccccgcgaggcgaaggttcagtggaaagtcgataatgcgttacagagcgggaatagccaggaaagtgttactgaacaggatagtaaagacagcacttacagcttatcgtctacacttactctgagtaaagcggattatgaaaagcataaggtgtacgcttgcgaggttacacatcaagggttatcttcgccagtcacgaagagctttaatcgcggcgagtgc

SEQ ID NO: 29 - DNA, 1347 bp, synthetic construct
  source 1..1347: mol_type = other DNA
caggtccagttggttgaaagtggcggcgggctcgtcaaaccaggaggctcacttcggctgagctgtgcggcgtccggtttcacttttagtgattactatatgtcctggatacggcaagcgcctgggaaaggtttggaatgggtgtcatatatttcatcaagcggatcaacaatctactatgctgactccgtgaaaggccggtttacaatcagcagagacaatgcaaaaaatagcctctatctccagatgaactctctgcgggcagaagatactgcagtgtattactgcgcccgagatttctacgatagtaagattttcgactattggggtcagggaaccttggttacagttagctcagcctccaccaagggcccaagtgtctttccgttagctccgtcttccaaaagtacgtcgggcggcacggcggcacttggctgcttagtcaaggactactttcccgaacccgttactgtctcttggaatagtggggctttgaccagtggggttcatacctttccagccgtacttcagtcttcagggctgtatagtttaagttcagtggttacagtgccctcttcatctttagggacccaaacctatatctgcaacgtcaatcataagccttcaaataccaaggttgacaaaaaggtggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 30 - DNA, 642 bp, synthetic construct
  source 1..642: mol_type = other DNA
gatatacaaatgacgcagtctcctagcagtctctccgcatccgtgggggaccgcgtaacaatcacttgtcgcgccagccagtcaatttcctcctaccttaattggtatcaacagaaaccgggaaaagccccgaaactcctgatttatgcagcttcatcactccagagtggcgtaccgtcaaggtttagtggttctggttctgggactgatttcacgctgacgatcagctctttgcaaccggaggatgtaggggtatattattgcatggaaggattgaaaacgccatttaccttcggaccggggacaaaactggaaattaaacgtacggtcgctgcgcccagtgtatttatctttcctcccagcgatgagcaactcaaaagtggaaccgccagtgtggtgtgtttgttgaataacttttatcccagagaagctaaggtacaatggaaggtcgataacgcccttcagtctggaaatagtcaggaaagtgtcacagagcaggattccaaggactccacgtatagtcttagtagtaccttgactctgagcaaagcagactatgagaagcacaaggtgtatgcttgtgaagtgacgcaccagggcctctcctcccccgtgacgaagagctttaatcgcggagagtgc

SEQ ID NO: 31 - protein, 6 aa, synthetic construct
HHHHHH

SEQ ID NO: 32 - protein, 120 aa, synthetic construct
QVQLEESGAELARPGSSVKLSCKASGYTFTNYWLQWVKQRTGQGLEWIGAIYPRDGDAKYSQKFKDKASLTVNESSSTAYMHLSALASEDSAVYYCARANYGLYYAMDRWGQGTSVTVSS

SEQ ID NO: 33 - protein, 107 aa, synthetic construct
DIFLTQSPANMSVSPGERVSFSCRASQNIGTNIHWYQQRTNGSPRLLIKYASESISGIPSRFSGSGSGTDFILSINTVESEDIAVYFCQQSNRWPFTFGSGTKLEVI

SEQ ID NO: 34 - protein, 4 aa, synthetic construct
QVQL

SEQ ID NO: 35 - protein, 4 aa, synthetic construct
GLEW

SEQ ID NO: 36 - protein, 7 aa, synthetic construct
AVYYCAR

SEQ ID NO: 37 - protein, 5 aa, synthetic construct
WGQGT

SEQ ID NO: 38 - protein, 5 aa, synthetic construct
VTVSS

SEQ ID NO: 39 - protein, 4 aa, synthetic construct
TQSP

SEQ ID NO: 40 - protein, 5 aa, synthetic construct
CRASQ

SEQ ID NO: 41 - protein, 4 aa, synthetic construct
WYQQ

SEQ ID NO: 42 - protein, 11 aa, synthetic construct
RFSGSGSGTDF

SEQ ID NO: 43 - protein, 5 aa, synthetic construct
PFTFG

SEQ ID NO: 44 - protein, 5 aa, synthetic construct
GTKLE

(19) United States
(12) Patent Application Publication
(10) Pub. No.: US 2026/0167677 A1
(43) Pub. Date: Jun. 18, 2026

(54) ANTI-ADAM10 ANTIBODIES AND USES IN TREATMENT OF CANCER

(71) Applicants: Memorial Sloan Kettering Cancer Center, New York, NY (US); Sloan Kettering Institute for Cancer Research, New York, NY (US); Memorial Hospital for Cancer and Allied Diseases, New York, NY (US); University of Pittsburgh - Of the Commonwealth System of Higher Education, Pittsburgh, PA (US)

(72) Inventors: Dimitar B. Nikolov, New York, NY (US); Dimiter Stanchev Dimitrov, St. Augustine, FL (US); Nayanendu Saha, New York, NY (US); Du-San Baek, Pittsburgh, PA (US)

(21) Appl. No.: 19/124,529

(22) PCT Filed: Oct. 28, 2023
(86) PCT No.: PCT/US2023/078131

§ 371 Date: Apr. 25, 2025

Related U.S. Application Data
(60) Provisional application No. 63/420,118, filed on Oct. 28, 2022.

(51) Int. Cl. C07K 14/005

(52) U.S. Cl. CPC C07K 14/005

(57) ABSTRACT

The present invention provides various ADAM10 binding molecules (including antibodies and fragments thereof), compositions comprising such ADAM10 binding molecules, and methods of using such ADAM10 binding molecules and compositions, for example in inhibiting binding of ADAM10 to ADAM10 substrates (such as Notch, epidermal growth factor receptor, or erythro-poietin-producing human hepatocellular receptor ligands), in inhibiting the proliferation of cancer cells, and in treating cancer.

### CROSS-REFERENCE TO RELATED APPLICATIONS

This application claims the benefit and priority of U.S. Provisional Application Ser. No. 63/420,118, filed on Oct. 28, 2022, the contents of each of which are herein incorporated by reference in their entirety.

### SEQUENCE LISTING

The instant application contains a Sequence Listing which has been submitted electronically in XML format and is hereby incorporated by reference in its entirety. The XML copy, created on Oct. 20, 2023, is named MSKCC059WO1.xml and is 45,577 bytes in size.

### BACKGROUND OF THE INVENTION

Colorectal cancer (CRC) is the third most commonly diagnosed cancer in the U.S. Current anti-colorectal treatments, predominantly relying on surgery, radiation, and conventional chemotherapeutics, are not curative and commonly result in drug-resistant disease relapse. Almost half of all colorectal cancer patients will develop recurrent disease. Surgically resected cases of CRC are known to have a 40%-60% recurrence rate in the first three years after surgery with the majority in the second year. Lymph node metastasis and/or adjacent organ involvement in stage II is said to have a recurrence of 20%-30% and stage III 50%-80% recurrence after surgery(Young et al., 2014).

A subgroup of cancer cells with stem cell-like properties, known as cancer stem cells (CSCs), initiate and sustain tumor growth and promote metastasis and chemoresistance in CRC. A key determinant of the CSC phenotype is activation of Notch receptor signaling. Ligand-activated Notch signaling involves sequential cleavage of the extracellular and intracellular domains by ADAM110 metalloprotease (functioning as alpha-secretase) and γ-secretase activity, respectively, to modulate downstream transcription of target genes (Hartmann et al., 2002). In addition to Notch receptor signaling, EGFR/erbB signaling that is essential for the development and metastasis of CRC, is also dependent on ADAM10 activity (Hartmann et al., 2013; Murphy, 2008). Notch signaling and CSCs are also associated with drug resistance, and inhibition of Notch signaling is widely reported to increase sensitivity to both chemo- and targeted therapies (Fischer et al., 2011; Li et al., 2011; Domingo-Domenech, et al. 2012; McAuliffe et al., 2012; Timme et al., 2013; Meng et al., 2009) However, targeting Notch using pan-specific γ-secretase inhibitors (Groth and Fortini, 2012) is hampered by intestinal toxicity, reflecting the diversity of γ-secretase targets (Dikic & Schmidt, 2010). Nevertheless. Notch-specific inhibitory monoclonal antibodies (mAbs), avoiding this toxicity, have validated Notch inhibition as a promising anti-cancer therapeutic approach (Wu et al., 2010).

ADAM (A Disintegrin And Metalloprotease) proteases consist of an N-terminal pro-sequence followed by metalloprotease (M), disintegrin (D), cysteine-rich (C), transmembrane, and cytoplasmic domains (Seals & Courtneidge 2003). The substrate specificity of ADAM proteases is not imparted by a typical substrate cleavage signature but relies on noncatalytic interactions between the substrate and the ADAM D+C domains (Janes et al., 2005; Wolfsberg & White, 2004; White, 2003; Reddy et al., 2000; Smith et al., 2002). ADAM10 is principally involved in activation of Notch (Andersson & Lendahl, 2014), and Eph (Janes et al., 2005; Janes et al., 2009) receptors signaling, while both ADAM10 and its close relative ADAM17 (TNFα-activating enzyme, or TACE) activate erbB/EGFR receptors via shedding of their ligands with differing specificities (Murphy, 2008). ADAM10 overexpression correlates with aberrant signaling from Notch, erbBs, and other receptors, as well as a more aggressive, metastatic phenotype in a range of cancers including colon, gastric, prostate, breast, ovarian, uterine, and leukemia (Gavert et al., 2007; Wang et al., 2011; Smith et al., 2020). As such. ADAM10 overexpression has been shown to induce metastases of human HCT116 CRC cells in mice (Gavert et al., 2007), suggesting that its inhibition may have potent antitumor effects.

However, attempts to inhibit ADAM10 with small-molecule inhibitors directed at its catalytic site failed clinical trials due to lack of specificity and efficacy, as well as dose-limiting toxicity. As such, there is a need in the art for new and improved strategies for ADAM10 inhibition. The present invention addresses this need.

### SUMMARY OF INVENTION

Some of the main aspects of the present invention are summarized below. Additional aspects are described in the Detailed Description of the Invention, Examples, Drawings, and Claims sections of this disclosure. The description in each section of this disclosure is intended to be read in conjunction with the other sections. Furthermore, the various embodiments described in each section of this disclosure can be combined in various different ways, and all such combinations are intended to fall within the scope of the present invention.

The invention provides novel protein targets that can be used in diagnostic and therapeutic applications for cancer.

In one aspect, the present invention relates to an ADAM10 binding molecule comprising: (a) a heavy chain variable region comprising: a complementarity determining region (CDR)-1 domain comprising an amino acid sequence of SEQ ID NO. 1, a CDR-2 domain comprising an amino acid sequence of SEQ ID NO. 2, and a CDR-3 domain comprising an amino acid sequence of SEQ ID NO. 3, and (b) a light chain variable region comprising: a CDR-1 domain comprising an amino acid sequence of SEQ ID NO. 4, a CDR-2 domain comprising an amino acid sequence of SEQ ID NO. 5, and a CDR-3 domain comprising an amino acid sequence of SEQ ID NO. 6.

In certain embodiments, in the heavy chain variable region, the CDR-1 domain consists of an amino acid sequence of SEQ ID NO. 1, the CDR-2 domain consists of an amino acid sequence of SEQ ID NO. 2, and the CDR-3 domain consists of an amino acid sequence of SEQ ID NO. 3; and in the light chain variable region, the CDR-1 domain consists of an amino acid sequence of SEQ ID NO. 4, the CDR-2 domain consists of an amino acid sequence of SEQ ID NO. 5, and the CDR-3 domain consists of an amino acid sequence of SEQ ID NO. 6.

In some embodiments, the heavy chain variable region comprises an amino acid sequence at least 95% identical to SEQ ID NO. 22, and the light chain variable region comprises an amino acid sequence at least 95% identical to SEQ ID NO. 23. In certain embodiments, the heavy chain variable region comprises an amino acid sequence of SEQ ID NO. 22, and the light chain variable region comprises an amino acid sequence of SEQ ID NO. 23. In particular embodiments, the heavy chain variable region consist of an amino acid sequence of SEQ ID NO. 22, and the light chain variable region consist of an amino acid sequence of SEQ ID NO. 23.

In another aspect, the present invention relates to an ADAM10 binding molecule comprising: heavy chain CDR-1, CDR-2, and CDR-3 domains that are contained within a heavy chain variable region comprising an amino acid sequence of SEQ ID NO. 22, and light chain CDR-1, CDR-2, and CDR-3 domains contained within a light chain variable region comprising an amino acid sequence of SEQ ID NO. 23 In some embodiments, the heavy chain CDR-1, CDR-2, and CDR-3 domains are contained within a heavy chain variable region consisting of an amino acid sequence of SEQ ID NO. 22, and the light chain CDR-1, CDR-2, and CDR-3 domains are contained within a light chain variable region consisting of an amino acid sequence of SEQ ID NO. 23.

In some embodiments, the ADAM 10 binding molecule comprises a heavy chain comprising an amino acid sequence of SEQ ID NO. 29, and a light chain comprising an amino acid sequence of SEQ ID NO. 30. In certain embodiments, the heavy chain consists of an amino acid sequence of SEQ ID NO. 29, and the light chain consists of an amino acid sequence of SEQ ID NO. 30.

In another aspect, the present invention relates an ADAM10 binding molecule that specifically binds to the same epitope on human ADAM10 as an ADAM10 binding molecule described herein.

In yet another aspect, the present invention relates an ADAM10 binding molecule that competes with an ADAM10 binding molecule described herein for binding to human ADAM10.

The ADAM10 binding molecule may be an antibody. In some embodiments, the antibody is a humanized antibody, a fully human antibody, a murine antibody, a chimeric antibody, a monoclonal antibody, a polyclonal antibody, a bi-specific antibody, or a multi-specific antibody. In certain embodiments, the binding molecule is a Fv, a Fab, a F(ab′)2, a Fab′, a dsFv fragment, a single chain Fv (scFV), an sc(Fv)2, a disulfide-linked (dsFv), a diabody, a triabody, a tetrabody, a minibody, or a single chain antibody.

In some embodiments, the ADAM10 binding molecule comprises a heavy chain constant region. In certain embodiments, the heavy-chain constant region is selected from the group consisting of alpha, delta, epsilon, gamma, and mu heavy chain constant regions.

In some embodiments, the binding molecule is an IgA, IgD, IgE, IgG or IgM class immunoglobulin.

In some embodiments, the ADAM10 binding molecule comprises a light chain constant region. In certain embodiments, the light chain constant region is a lambda light chain constant region or a kappa light chain constant region.

In a further aspect, the present invention relates to a composition comprising an ADAM10 binding molecule of the invention.

In another aspect, the present invention relates to a pharmaceutical composition comprising an ADAM10 binding molecule of the invention.

In another aspect, the present invention relates to an isolated nucleic acid molecule comprising a nucleotide sequence encoding an ADAM10 binding molecule of the invention.

In yet another aspect, the present invention relates to a vector comprising a nucleic acid molecule, in which the nucleic acid molecule comprises a nucleotide sequence encoding an ADAM10 binding molecule of the invention.

In a further aspect, the present invention relates to a host cell that produces an ADAM10 binding molecule of the invention, or that comprises a nucleic acid molecule of the invention, or that comprises a vector of the invention. In some embodiments, the cell is a mammalian cell. In certain embodiments, the cell is a human cell. In other embodiments, the cell is a murine cell.

In another aspect, the present invention relates to a method for inhibiting the proliferation of, or killing, tumor cells, the method comprising delivering to tumor cells an effective amount of an ADAM10 binding molecule of the invention, a composition of the invention, or a pharmaceutical composition of the invention. In some embodiments, the tumor cells are selected from the group consisting of colorectal cancer cells, colon cancer cells, breast cancer cells, ovarian cancer cells, lung cancer cells, non-small cell lung cancer cells, brain cancer cells, glioma cells, glioblastoma cells, and neuroblastoma cells. In certain embodiments, the tumor cells overexpress, exhibit over-activity of, or are dependent on signaling of, Notch, epidermal growth factor receptor (EGFR), or erythropoietin-producing human hepatocellular (Eph) receptor. The tumor cells may be in vitro or in vivo.

In another aspect, the present invention relates to a method of inhibiting a biological activity in cells or in a tissue, in which the biological activity is selected from the group consisting of: (a) binding of ADAM10 to an ADAM10 substrate, (b) proteolytic cleavage of an ADAM10 substrate by ADAM10, (c) activation of an ADAM10 substrate, and (d), signaling by an ADAM10 substrate, the method comprises delivering an effective amount of an ADAM10 binding molecule of the invention, or a composition of the invention, or a pharmaceutical composition of the invention, to cells or a tissue that expresses or contains ADAM10, thereby inhibiting the biological activity in the cells or tissue. In some embodiments, the cells or tissue are selected from the group consisting of colorectal cancer cells or tissue, colon cancer cells or tissue, breast cancer cells or tissue, ovarian cancer cells or tissue, lung cancer cells or tissue, non-small cell lung cancer cells or tissue, brain cancer cells or tissue, glioma cells or tissue, glioblastoma cells or tissue, and neuroblastoma cells or tissue. The cells or tissue are in vitro, or in vivo.

In some embodiments, the ADAM10 substrate is a ligand of Notch, EGFR, or Eph receptor.

In yet a further aspect, the present invention relates to a method of treating cancer in a subject, the method comprising administering to a subject having cancer an effective amount of an ADAM10 binding molecule of the invention, a composition of the invention, or a pharmaceutical composition of the invention. In some embodiments, the cancer is selected from the group consisting of colorectal cancer, colon cancer, breast cancer, ovarian cancer, lung cancer, non-small cell lung cancer, brain cancer, glioma, glioblastoma, and neuroblastoma. In some embodiments, the cancer comprises tumor cells that overexpress, exhibit over-activity of, or are dependent on signaling of, Notch, EGFR, or Eph receptor.

In some embodiments, the method further comprises administering an additional active agent to the subject. In some embodiments, the additional active agent is a chemotherapeutic agent. In other embodiments, the additional active agent is an antibody, or antigen binding fragment thereof. In certain embodiments, the additional active agent is selected from the group consisting of afatinib, actinomycin, azacitidine, azathioprine, bevacizumab, bleomycin, bortezomib, carboplatin, capecitabine, cetuximab, cisplatin, chlorambucil, cyclophosphamide, cytarabine, daunorubicin, docetaxel, doxifluridine, doxorubicin, epirubicin, epothilone, erlotinib, etoposide, fluorouracil, gefitinib, gemcitabine, hydroxy urea, idarubicin, imatinib, irinotecan, mechlorethamine, mercaptopurine, methotrexate, mitoxantrone, olaparib, oxaliplatin, paclitaxel, panitumab, pazopanib, pemetrexed, poly(ADP-ribose) polymerase (PARP) inhibitors, tamoxifen, teniposide, tioguanine, topotecan, trastuzumab, tretinoin, valrubicin, vinblastine, vincristine, vndesine, vinorelbine, and vintafolide.

In another aspect, the present invention relates to a method for detecting ADAM10 in a sample, the method comprising (a) contacting a sample with an ADAM10 binding molecule of the invention, a composition of the invention, or a pharmaceutical composition of the invention; and (b) detecting binding of the ADAM10 binding molecule to ADAM10, thereby detecting ADAM10 in the sample.

In yet another aspect, the present invention relates to a method of determining whether a subject with a tumor is a candidate for treatment with an ADAM10 binding molecule, the method comprising: (a) contacting a tumor sample from a subject, or cells therefrom, with an ADAM10 binding molecule of the invention, or a composition of the invention, or a pharmaceutical composition of the invention; and (b) performing an assay to determine whether the ADAM10 binding molecule binds to ADAM10 in the sample; whereby if the ADAM10 binding molecule binds to ADAM10 in the sample the subject is a candidate for treatment with an ADAM10 binding molecule of the invention, or a composition of the invention, or a pharmaceutical composition of the invention.

In a further aspect, the present invention relates to a method of determining whether a subject with a tumor is a candidate for treatment with an ADAM10 binding molecule, the method comprising: (a) contacting a tumor sample from a subject, or cells therefrom, with an ADAM10 binding molecule of the invention, or a composition of the invention, or a pharmaceutical composition of the invention; and (b) performing an assay to determine whether the ADAM10 binding molecule inhibits proteolytic cleavage of an ADAM10 substrate in the sample; whereby if the ADAM10 binding molecule inhibits proteolytic cleavage of the ADAM10 substrate in the sample the subject is a candidate for treatment with an ADAM10 binding molecule of the invention, or a composition of the invention, or a pharmaceutical composition of the invention.

In yet a further aspect, the present invention relates to a method of determining whether a subject with tumor is a candidate for treatment with an ADAM10 binding molecule, the method comprising: (a) contacting a tumor sample from a subject, or cells therefrom, with an ADAM10 binding molecule of the invention, or a composition of the invention, or a pharmaceutical composition of the invention; and (b) performing an assay to determine whether the ADAM10 binding molecule inhibits activation of or signaling of an ADAM10 substrate in the sample; whereby if the ADAM10 binding molecule inhibits activation or signaling of the ADAM10 substrate in the sample the subject is a candidate for treatment with an ADAM10 binding molecule of the invention, or a composition of the invention, or a pharmaceutical composition of the invention.

In some embodiments, the methods further comprise administering an effective amount of an ADAM10 binding molecule of the invention, or a composition of the invention, or a pharmaceutical composition of the invention, to the subject.

In some embodiments, the tumor is selected from the group consisting of colorectal tumor, colon tumor, breast tumor, ovarian tumor, lung tumor, non-small cell lung tumor, brain tumor, glioma, glioblastoma, and neuroblastoma. In certain embodiments, the ADAM10 substrate is a ligand of Notch, EGFR, or Eph receptor.

### BRIEF DESCRIPTION OF THE DRAWING FIGURES

FIG. 1 shows SDS-PAGE profile of the purified bovine (b) ADAM10 disintegrin+cysteine-rich domain construct (D+C), as described in Example 1.

FIG. 2 shows results associated with the generation of a fully human anti-ADAM10 antibody, named “1H5.” as described in Example 2. Panels A-C show characterization of the fully human 1H5 Fab. Panel A shows binding profile of individual Fab binders to ADAM10 D+C, and Panel B shows binding of 1H5 Fab to ADAM10 D+C. Panel C shows results of competitive enzyme-linked immunosorbent assay (ELISA) with the murine monoclonal antibody (mAb) “8C7.” which demonstrates that 1H5 Fab (15 nM fixed concentration) binds to a similar ADAM10 epitope. Panel D shows results of a pull-down experiment, demonstrating that ADAM10 D+C (b) binds to protein A Sepharose bead-bound 8C7 and 1H5. [Lane 1: Low molecular weight standards (Bio-Rad). Lane 2: bead bound murine 8C7 immunoglobulin G (IgG)+ADAM10 D+C. Lane 3: bead bound 1H5 IgG+ ADAM10 D+C. Lane 4: bead bound ADAM10 D+C without any pre-bound mAb. Lane 5: ADAM10 D-f-C input] Panel E shows ELISA results, which demonstrate that 1H5 IgG binds specifically to immobilized human (h) and bovine (b) ADAM10 D-C, but not to human ADAM17 D+C or human ADAM19 D+C.

FIG. 3 shows results of a competitive ELISA that gauged relative bindings of 1115 and 8C7 to immobilized ADAM10 D+C domains (antigen), as described in Example 2. “1H5/anti-murine secondary” antibody represents 1H5 binding to ADAM10 D+C detected with rabbit anti-mouse IgG cross-adsorbed secondary antibody, while “8C7/anti-human secondary” antibody represents 8C7 binding to ADAM10 D+C detected with goat anti-human IgG cross-adsorbed secondary antibody. “1H5:8C7(1:1)/anti-murine secondary” represents the binding of 8C7 to the antigen when added in a 1:1 ratio with 1H5, while “1H5:8C7(1:1)/anti-human secondary” shows the binding of 1H5 to the antigen when added in a 1:1 ratio with 8C7. “Murine 8C7/anti-murine secondary” depicts the binding of 8C7 to the antigen, while “human 1H5/anti-human secondary” refers to the 1H5 binding to the antigen, detected using anti-mouse/human secondary, when the 1H5 or 8C7 are added individually. The data represent mean of triplicate determinations and two independent experiments, mean±SEM; P<0.001 by unpaired two-tailed Student's t-test (mAbs binding to antigen vs uncoated well control).

FIG. 4 shows results of Alamar blue cell viability assays with multiple cancer cell lines, as described in Example 3. Percent growth inhibition is shown for the following cell lines treated with 1H5: colon cancer cell lines COLO205 and LIM1215 (Panel A), and HT29 and HCT116 (Panel E); breast cancer cell lines MDA-MB-231 and SKBR-3 (Panel B); ovarian cancer cell lines SKOV-3 and OVCAR-3 (Panel C); and glioblastoma cell line U87-MG and non-small cell lung cancer cell line HCC-827 (Panel D). The data represent mean of triplicate determinations and two independent experiments, and the bar plots show the effect of treatment of mAbs on cancer cells relative to the control, mean±SEM.

FIG. 5 shows the effects of 1H5 on tumor volume in a colon cancer xenograft model, as described in Example 4. In a first study, the mice were administered 1HF alone (Group 1), irinotecan (Group 2), 1H5 in combination with irinotecan (Group 3), or sterile phosphate buffer solution (control) (Group 4). Panel A shows average tumor volume (±SD) in the mice from day 7 to 35, and Panel B shows a box-and-whiskers plot of the average tumor volume at day 35 (horizontal lines indicate the average value; top and bottom of the boxes indicate the interquartile range; and whiskers indicate the range). In a second study, the mice were administered 1HF at a 20 mg/kg dose (Group 1) or at a 10 mg/kg dose (Group 2), or sterile phosphate buffer solution (control) (Group 3). Panel C shows average tumor volume in the mice from day 9 to 35, and Panel D shows the weight of the mice from day 9 to day 35.

FIG. 6 shows a crystal structure of the 1H5/ADAM10 D+C complex, as described in Example 5. Panel A shows the overall structure of the ADAM10 D+C/1H5 complex shown in ribbon view, as well as binding interface comparison (zoom-in insets) with the ADAM10 D+C/8C7 structure showing a similar epitope region with distinct recognition strategies (see also FIG. 7 for epitope comparison). Panel B shows superimposition of 1H5 and 8C7, in complex with the ADAM10 D+C domain, illustrating the different antibody approaching angles. ADAM10 is shown as ribbons (green: 1H5 bound; grey: 8C7 bound). The antibodies are shown as molecular surfaces (1H5: nontransparent orange and blue; 8C7: semi-transparent yellow and cyan). Panel C shows an overlay of the ADAM10 D+C (in green)/1H5 (in orange and blue surface representation) and the ADAM10 extracellular domain (ECD) (in magenta) structures showing a partial overlay (indicated with the black circle) of 1H5 with the M domain in the autoinhibited ADAM10 conformation. (Sec also Saha et al., 2023, which is incorporated herein by reference).

FIG. 7 shows antibody epitope and sequence analysis of 1H5 and 8C7, as described in Example 5. Panel A shows antibody epitope (in red) on ADAM10. The ADAM10 D+C domain structures are illustrated in ribbons with transparent surfaces. [1H5 (top, green), 8C7 (bottom, grey)] Panel B (SEQ ID NOS: 7, 8, and 32-44) shows sequence alignment between 1H5 and 8C7. [Heavy chain (top), light chain (bottom)]. (See also Saha et al., 2023, which is incorporated herein by reference).

FIG. 8 shows results of cellular ELISA assay that gauged the binding of 1H5, relative to the binding of the control anti-ADAM10 mAb1427 (“mAb1427”) (Panel A) or 4A11 (Panel B), to ADAM10 expressed on the cell surface of colon cancer cell lines LIM1215. COLO205, as well as HEK293 cells and HEK293 cells transfected with human ADAM10, as described in Example 6. The graph show the 1H5/mAb1427 signal ratio observed for the noted cell line relative to the 1H5/mAb1427 signal ratio observed for the untransfected HEK293 cells. Specifically, on the Y axes is plotted the value of:

$$\frac{A ( 1 H 5 ) / A ( mAb 1427 )}{A ( 1 H 5 - HEK ) / A ( mAb 1427 - HEK )}$$

where A(1H5-HEK) is the signal for 1H5 using the untransfected HEK293 cells; A(mAb1427-HEK) is the signal for the mAb1427 using the untransfected HEK293 cells; A(1H5) is the signal for 1H5 using the cells that are being evaluated; and A(mAb1427) is the signal for mAb1427 using the cells being evaluated. The data represent triplicate determinations and two independent experiments, mean±SEM; P<0.001 by unpaired two-tailed Student's t-test (cancer cell lines vs HEK293 cells).

FIG. 9 shows the effect of 1H5 on ADAM10 catalytic activity using a fluorogenic peptide cleavage assay, as described in Example 7. Panel A shows SDS-PAGE profile of purified human (h) and bovine (b) catalytically active ADAM10 ECD Panels B and C show FRET-based peptide cleavage assays. The data represent mean of triplicate determinations and two independent experiments. Representative results of triplicate determinations and two independent experiments show the effects of mAbs or the metalloprotease inhibitor GM6001 on substrate cleavage relative to the control (ADAM10 without the mAbs/inhibitor); mean±SEM; P<0.001 by unpaired two-tailed Student's t test, (ADAM10 with mAb/inhibitor vs ADAM10 without mAb/inhibitor).

FIG. 10 shows schematic representation of a proposed mechanism for ADAM10 activation and interactions with substrates and the 1H5 antibody, as described in Example 7.

FIG. 11 shows results of Sandwich ELISA that was used to measure the levels of total (Panel A) and cleaved (Panel B) Notch1 in COLO205 cells upon treatment with 1H5, and described in Example 8. The data represent mean of triplicate experiments, and the bar plots show the effect of treatment with 1H5 relative to untreated control, mean±SEM. Comparison of notch levels between treated and untreated groups was performed using independent t test.

FIG. 12 shows results of Western blot analysis of 1H5 or its Fab fragment on amyloid precursor protein (APP) shedding. Panels A and B show images of the Western blot gels used to analyze 1H5 (Panel A) of its Fab fragment (Panel B). Lanes 1-8 of each gel are as follows: Lane 1=vector control; Lane 2=APP transfected, untreated; Lane 3=31 nM 1H5/Fab fragment; Lane 4=62 nM 1H5/Fab fragment; Lane 5=125 nM 1H5/Fab fragment; Lane 6=250 nM 1H5/Fab fragment; Lane 7=500 nM 1H5/Fab fragment; Lane 8=1 μM 1H5/Fab fragment. Pane C shows quantitation of sAPPα (˜100 kDa) shedding in the presence of the 11H5 and its Fab fragment.

### DETAILED DESCRIPTION OF THE INVENTION

The practice of the present invention can employ, unless otherwise indicated, conventional techniques of cell biology, cell culture, molecular biology, transgenic biology, microbiology, recombinant DNA, and immunology, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their Systeme International d'Unités (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value (i.e., intermediate) encompassed by the range, including integers and fractions. For example, a stated range of 5-10 is also a disclosure of 5, 6, 7, 8, 9, and 10 individually, and of 5.2, 7.5, 8.7, and so forth.

Unless otherwise indicated, the terms “at least” or “about” preceding a series of elements is to be understood to refer to every element in the series. The term “about” preceding a numerical value includes t 10% of the recited value. For example, a concentration of about 1 mg/mL includes 0.9 mg/mL to 1.1 mg/mL. Likewise, a concentration range of about 1% to 10% (w/v) includes 0.9% (w/v) to 11% (w/v).

Amino acids are referred to herein by their commonly known three-letter symbols or by the one-letter symbols recommended by the IUPAC-IUB Biochemical Nomenclature Commission. Nucleotides, likewise, are referred to by their commonly accepted single-letter codes.

The term “antibody” refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. The terms “antibody” or “immunoglobulin” are used interchangeably herein.

A typical antibody comprises at least two heavy chains and two light chains interconnected by disulfide bonds. Each heavy chain is comprised of a heavy chain variable region and a heavy chain constant region. The heavy chain constant region is comprised of three domains. CH1, CH2, and CH3. Each light chain is comprised of a light chain variable region and a light chain constant region (CL). The light chain constant region is comprised of one domain, Cl. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

Antibodies can be of any the five major classes of immunoglobulins: IgA, IgD, IgE, IgG and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. There are two classes of mammalian light chains, lambda and kappa.

The heavy and light chain variable regions can be further subdivided into regions of hypervariability, termed complementarity-determining regions (CDRs), interspersed with regions that are more conserved, termed framework (FW) regions. The CDRs in each chain are held together in close proximity by the FW regions and, with the CDRs from the other chain, contribute to the formation of the antigen-binding site of antibodies. Each heavy and light chain variable region is composed of three CDRs and four FW regions, arranged from amino-terminus to carboxy-terminus in the following order: FW-1, CDR-1, FW-2, CDR-2, FW-3, CDR-3, FW-4.

There are at least two techniques for determining CDRs: (1) an approach based on cross-species sequence variability (Kabat et al., 1991); and (2) an approach based on crystallographic studies of antigen-antibody complexes (Al-Lazikani et al., 1997). In addition, combinations of these two approaches are sometimes used in the art to determine CDRs.

The amino acid position numbering as in Kabat, refers to the numbering system used for heavy chain variable domains or light chain variable domains (approximately residues 1-107 of the light chain and residues 1-113 of the heavy chain). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a, according to Kabat) after residue 52 of H2 and inserted residues (eg., residues 82a, 82b, and 82c, etc., according to Kabat) after heavy chain FW residue 82.

The Kabat numbering of residues can be determined for a given antibody by alignment at regions of homology of the sequence of the antibody with a “standard” Kabat numbered sequence. Chothia refers instead to the location of the structural loops (Chothia & Lesk, 1987). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

IMGT (ImMunoGeneTics) also provides a numbering system for the immunoglobulin variable regions, including the CDRs (see. e.g., Lefranc et al., 2003). The IMGT numbering system was based on an alignment of more than 5,000 sequences, structural data, and characterization of hypervariable loops and allows for easy comparison of the variable and CDR regions for all species. According to the IMGT numbering schema heavy chain variable region CDR-1 is at positions 26 to 35, heavy chain variable region CDR-2 is at positions 51 to 57, heavy chain variable region CDR-3 is at positions 93 to 102, light chain variable region CDR-1 is at positions 27 to 32, light chain variable region CDR-2 is at positions 50 to 52, and light chain variable region CDR-3 is at positions 89 to 97.

As used herein, the term “antibody” encompasses polyclonal antibodies; monoclonal antibodies; multispecific antibodies, such as bispecific antibodies generated from at least two intact antibodies; humanized antibodies; human antibodies; chimeric antibodies; fusion proteins comprising an antigen-determination portion of an antibody; and any other modified immunoglobulin molecule comprising an antigen recognition site, so long as the antibodies exhibit the desired biological activity.

A “monoclonal antibody” (mAb) refers to a homogeneous antibody population that is involved in the highly specific recognition and binding of a single antigenic determinant, or epitope. This is in contrast to polyclonal antibodies, which typically include different antibodies directed against different antigenic determinants. The term “monoclonal” can apply to both intact and full-length monoclonal antibodies, as well as to antibody fragments (such as Fab. Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, by hybridoma, phage selection, recombinant expression, and transgenic animals.

The term “humanized antibody” refers to an antibody derived from a non-human (e.g., murine) immunoglobulin, which has been engineered to contain minimal non-human (e.g., murine) sequences. Typically, humanized antibodies are human immunoglobulins in which residues from the CDR are replaced by residues from the CDR of a non-human species (e.g., mouse, rat, rabbit, or hamster) that have the desired specificity, affinity, and capability (Jones et al., 1986; Riechmann et al., 1998; Verhoeyen et al., 1988). In some instances, the Fv FW residues of a human immunoglobulin are replaced with the corresponding residues in an antibody from a non-human species that has the desired specificity, affinity, and capability.

Humanized antibodies can be further modified by the substitution of additional residues either in the Fv framework region and/or within the replaced non-human residues to refine and optimize antibody specificity, affinity, and/or capability. In general, humanized antibodies will comprise substantially all of at least one, and typically two or three, variable domains containing all or substantially all of the CDR regions that correspond to the non-human immunoglobulin whereas all or substantially all of the FR regions are those of a human immunoglobulin consensus sequence. Humanized antibody can also comprise at least a portion of an immunoglobulin constant region or domain (Fc), typically that of a human immunoglobulin. Examples of methods used to generate humanized antibodies are described in U.S. Pat. Nos. 5,225,539 and 5,639,641.

The term “human antibody” means an antibody produced by a human or an antibody having an amino acid sequence corresponding to an antibody produced by a human made using any technique known in the art. The definition of a human antibody includes intact or full-length antibodies comprising at least one human heavy and/or light chain polypeptide such as, for example, an antibody comprising murine light chain and human heavy chain polypeptides.

The term “chimeric antibodies” refers to antibodies wherein the amino acid sequence of the immunoglobulin molecule is derived from two or more species Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

The term “antigen-binding fragment” refers to a portion of an intact antibody comprising the complementarity determining variable regions of the antibody. Examples of antibody fragments that can constitute an “antigen-binding fragment” include, but are not limited to. Fab. Fab′, F(ab′)2, and Fv fragments, linear antibodies, single chain antibodies (e.g., ScFvs), and multi-specific antibodies formed from antibody fragments.

A “blocking” antibody or an “antagonist” antibody is one that inhibits or reduces biological activity of the antigen it binds, such as ADAM10. In certain embodiments, blocking antibodies or antagonist antibodies substantially or completely inhibit the biological activity of the antigen. Desirably, the biological activity is reduced by 10%, 20%, 30%, 50%, 70%, 80%, 90%, 95%, or even 100%.

The term “germlining” means that amino acids at specific positions in an antibody are mutated back to those in the germ line.

The “IgG1 triple mutant” or “IgG1-TM” antibody format is a human IgG1 isotype containing three single amino acid substitutions, L234F/L235E/P331 S, within the lower hinge and CH2 domain (Oganesyan et al., 2008). The TM causes a profound decrease in binding to human FcγRI, FcγRII, FcγRIII, and Clq, resulting in a human isotype with very low effector function.

The terms “YTE” or “YTE mutant” or “YTE mutation” refer to a mutation in IgG1 Fc that results in an increase in the binding to human FcRn and improves the serum half-life of the antibody having the mutation. A YTE mutant comprises a combination of three mutations, M252Y/S254T/T256E (EU numbering Kabat et al., 1991), introduced into the heavy chain of an IgG1 (see U.S. Pat. No. 7,658,921, which is incorporated by reference herein). The YTE mutant has been shown to increase the serum half-life of antibodies approximately four-times as compared to wild-type versions of the same antibody (Dall' Acqua et al., 2002; Dall'Acqua et al., 2006; Robbie et al., 2013; see also U.S. Pat. No. 7,083,784, which is hereby incorporated by reference in its entirety).

The term “chimeric antigen receptor T cell” or “CART T cell” refers to a T cell that is genetically modified by adding a gene for a chimeric antigen receptor (CAR). The CAR helps the T cell target and attach to a specific antigen. A CAR is composed of four regions: an antigen recognition domain, which is responsible for targeting the CAR T cell to any cell expressing a specific molecule; a transmembrane domain, which is a structural component that spans the cell membrane of the T cell and anchors the CAR to the membrane; an extracellular hinge region, which is a spacer domain between the antigen recognition domain and the transmembrane domain; and an intracellular T cell signaling domain, which lies inside the T cell and perpetuates signaling in the T cell when the antigen recognition domain binds to an antigen.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer.

The affinity or avidity of an antibody for an antigen can be determined experimentally using an suitable method known in the art, e.g., flow cytometry, enzyme-linked immunosorbent assay (ELISA), or radioimmunoassay (RIA), or kinetics (e.g., KINEXA® or BIACORE™ or OCTET® analysis). Direct binding assays as well as competitive binding assay formats can be readily employed (see, e.g., Berzofsky et al., 1984; Kuby, 1992). The measured affinity of a particular antibody-antigen interaction can vary if measured under different conditions (e.g., salt concentration. pH, temperature). Thus, measurements of affinity and other antigen-binding parameters (e.g., K_{D} or K_{d}, Kₒₙ, K_{off}) are made with standardized solutions of antibody and antigen, and a standardized buffer, as known in the art.

The terms “inhibit,” “block,” and “suppress” are used interchangeably and refer to any statistically significant decrease in a given biological activity, including full blocking of the activity. For example, “inhibition” can refer to a decrease of about 10%, 20%, 30%, 40%, 50%, 60%, 70%, 80%, 90% or 100% in biological activity. Accordingly, when the terms “inhibition” or “suppression” are applied to describe, e.g., an effect of an ADAM10 binding molecule, the terms may refer to the ability of an ADAM10 binding molecule to statistically significantly decrease: (a) binding of ADAM10 to an ADAM10 substrate (such as Notch, ephrin, and EGFR ligands), or (b) proteolytic cleavage of an ADAM10 substrate by ADAM10, or (c) activation of, or signaling by, an ADAM10 substrate, or (d) proliferation or survival of a tumor cell whose proliferation or survival is driven, in part, by an ADAM10 substrate, and the like. Inhibition may be determined relative to an untreated control—for example, a control not treated with the ADAM10 binding molecule. In some embodiments, an ADAM10 binding molecule can inhibit an activity of ADAM10 (such as those listed above) by at least 10%, or at least 20%, or at least 30%, or at least 40%, or at least 50%, or at least 60%, or at least 70%, or at least 80%, or at least 90% or about 100%, as determined, for example, by flow cytometry. Western blotting, ELISA, proliferation assays, or other assays known to those of skill in the art.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, bears, and so on.

The term “pharmaceutical composition” refers to a preparation that is in such form as to permit the biological activity of the active ingredient to be effective and which contains no additional components that are unacceptably toxic to a subject to which the composition would be administered. Such composition can be sterile and can comprise a pharmaceutically acceptable carrier, such as physiological saline. Suitable pharmaceutical compositions can comprise one or more of a buffer (e.g., acetate, phosphate or citrate buffer), a surfactant (e.g., polysorbate), a stabilizing agent (e.g., human albumin), a preservative (e.g., benzyl alcohol), an absorption promoter to enhance bioavailability and/or other conventional solubilizing or dispersing agents.

An “effective amount” of a binding molecule as disclosed herein is an amount sufficient to carry out a specifically stated purpose. An “effective amount” can be determined empirically and in a routine manner, in relation to the stated purpose.

The ADAM10 binding molecules of the invention can be naked or conjugated to other molecules such as toxins, labels, etc. The term “label” when used herein refers to a detectable compound or composition that is conjugated directly or indirectly to a binding molecule, so as to generate a “labeled” binding molecule. The label can be detectable by itself (e.g., radioisotope labels or fluorescent labels) or, as in the case of, for instance, an enzymatic label, can catalyze chemical alteration of a substrate compound or composition that is detectable.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already with the disorder. In certain embodiments, a subject is successfully “treated” for a disease or disorder according to the methods provided herein if the patient shows, e.g., total, partial, or transient alleviation or elimination of symptoms associated with the disease or disorder.

“Prevent” or “prevention” refer to prophylactic or preventative measures that prevent and/or slow the development or recurrence of a targeted pathologic condition or disorder. Thus, those in need of prevention include those prone to have or susceptible to the disorder, including those who have had the disorder and are susceptible to recurrence. In certain embodiments, a disease or disorder is successfully prevented according to the methods provided herein if the patient develops, transiently or permanently, e.g., fewer or less severe symptoms or pathology associated with the disease or disorder, or a later onset of symptoms or pathology associated with the disease or disorder, than a patient who has not been subject to the methods of the invention. In some embodiments, recurrence of cancer is prevented for at least about 3, 6, 9, 12, 18, or 24 months after the start of treatment with an ADAM10 binding molecule of the invention.

The terms “polypeptide,” “peptide.” and “protein” are used interchangeably herein to refer to polymers of amino acids of any length. The polymer can be linear or branched, it can comprise modified amino acids and non-amino acids can interrupt it. The terms also encompass an amino acid polymer that has been modified naturally or by intervention; for example, disulfide bond formation, glycosylation, lipidation, acetylation, phosphorylation or any other manipulation or modification such as conjugation with a labeling component. Also included within the definition are, for example, polypeptides containing one or more analogs of an amino acid (including, for example, unnatural amino acids, etc.), as well as other modifications known in the art. In certain embodiments, the polypeptides can occur as single chains or associated chains.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., asparagme, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the amino acid sequences of the binding molecules of the invention do not abrogate the binding of the binding molecule to the antigen(s), i.e., ADAM10, to which the binding molecule binds. Methods of identifying conservative nucleotide and amino acid substitutions which do not eliminate antigen-binding are well-known in the art (see, e.g., Brummell et al, 1993; Kobayashi et al, 1999; Burks et al., 1997).

A “polynucleotide,” as used herein can include one or more “nucleic acids,” “nucleic acid molecules,” or “nucleic acid sequences,” and refers to a polymer of nucleotides of any length, and includes DNA and RNA. The polynucleotides can be deoxyribonucleotides, ribonucleotides, modified nucleotides or bases, and/or their analogs, or any substrate that can be incorporated into a polymer by DNA or RNA polymerase A polynucleotide can comprise modified nucleotides, such as methylated nucleotides and their analogs. The preceding description applies to all polynucleotides referred to herein, including RNA and DNA.

The term “vector” means a construct, which is capable of delivering and, in some embodiments expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

An “isolated” polypeptide, antibody, binding molecule, polynucleotide, vector, or cell is in a form not found in nature. Isolated polypeptides, antibodies, binding molecules, polynucleotides, vectors, or cells include those which have been purified to a degree that they are no longer in a form in which they are found in nature. In some embodiments, a polypeptide, antibody, binding molecule, polynucleotide, vector, or cell that is isolated is substantially pure. When used herein, the term “substantially pure” refers to purity of greater than 75%, preferably greater than 80% or 90%, and most preferably greater than 95%.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

One such non-limiting example of a sequence alignment algorithm is the algorithm described in Karlin & Altschul (1990), as modified in Karlin et al. (1993), and incorporated into the NBLAST and XBLAST programs (Altschul et al., 1997). In certain embodiments, Gapped BLAST can be used as described in Altschul et al. (1997). BLAST-2, WU-BLAST-2 (Altschul & Gish, 1996). ALIGN, ALIGN-2 (Genentech, South San Francisco, California) or Megalign (DNASTAR) are additional publicly available software programs that can be used to align sequences. In certain embodiments, the percent identity between two nucleotide sequences is determined using the GAP program in the GCG software package (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (1970), can be used to determine the percent identity between two amino acid sequences (e.g., using either a BLOSUM 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS 4:11-17 (1989)). For example, the percent identity can be determined using the ALIGN program (version 2.0) and using a PAM120 with residue table, a gap length penalty of 12 and a gap penalty of 4. One skilled in the art can determine appropriate parameters for maximal alignment by particular alignment software. In certain embodiments, the default parameters of the alignment software are used.

In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

The term “ALAMARBLUE” as used herein refers to the cell viability reagent that is commercially available from ThermoFischer Scientific (catalog no. DALI 100) and that is described in Back et al. (1999).

Other terms are defined elsewhere in this patent disclosure, or else are used in accordance with their usual meaning in the art.

### ADAM10 Binding Molecules

The acronym “ADAM“refers to” a disintegrin and Metalloproteinase” enzyme. ADAMs are Zn²⁺-dependent, modular cell surface proteins that belong to the adamalysin protein family. ADAM10 is also referred to using synonyms: CD156c, CDw156, AD10, AD18, HsT18717, MADM, RAK, kuz). The ADAM10 protein, and the nucleotide sequences that encode it are well known in the art, and both the nucleotide and amino acid sequences of ADAM10s from several different species (including humans and mice) are publicly available, for example in the GenBank/NCBI database.

The present invention provides ADAM10 binding molecules, e.g., anti-ADAM10 antibodies, and antigen-binding fragments thereof, which specifically bind ADAM10.

The terms “ADAM10 binding molecule” or “binding molecule that binds to ADAM10” or “anti-ADAM10” refer to a binding molecule that is capable of binding ADAM10 with sufficient affinity such that the binding molecule is useful for one of the applications described herein, including, but not limited to, in inhibiting binding of ADAM10 to ADAM10 substrates (such as Notch, ephrin, and EGFR ligands), inhibiting ADAM10-dependent proteolytic cleavage of ADAM10 substrates (such as Notch, ephrin, and EGFR ligands), inhibiting activation of ADAM10 substrates (such as Notch, ephrin, and EGFR ligands, or inhibiting tumor cell proliferation in vitro or in vivo, for example in therapeutic applications. Typically, a binding molecule that “specifically binds” to ADAM10 binds to an unrelated, non-ADAM10 protein to an extent of less than about 10% of the binding of the binding molecule to ADAM10, as measured, for instance, by a radioimmunoassay (RIA), BIACORE™ (e.g., using recombinant ADAM10 as the analyte and binding molecule as the ligand, or vice versa), KINEXA®, OCTET®, or other binding assays known in the art. In certain embodiments, binding molecule that binds to ADAM10 has a dissociation constant (K_{D}) of ≤1 μM, ≤100 nM, 510 nM, 51 nM, ≤0.1 nM, ≤10 μM, 1 μM, or ≤0.1 μM.

Exemplary ADAM10 binding molecules of the present invention include the antibody clone referred to herein as “1H5” and antigen binding fragments thereof, such as antigen binding fragments that comprise the CDRs of these lead antibody clones. The amino acid sequences for the CDRs, heavy chain and light chain variable regions, and full heavy and light chains of 1H5 are provided in Table 1, which also provides SEQ ID NOs for each amino acid sequence.

TABLE 1
Amino acid sequences of 1H5 CDRs (according to Kabat numbering), heavy and light chain
variable regions. and full heavy and light chains.

| Region/Component | SEQ ID NO | Amino Acid Sequence |
|---|---|---|
| Heavy Chain CDR-1 | SEQ ID NO: 1 | DYYMS |
| Heavy Chain CDR-2 | SEQ ID NO: 2 | YISSSGSTIYYADSVKG |
| Heavy Chain CDR-3 | SEQ ID NO: 3 | DFYDSKIFDY |
| Heavy Chain | SEQ ID NO: 7 | QVQLVESGGGLVKPGGSLRLSCAASGFTF |
| Variable Region | | SDYYMSWIRQAPGKGLEWVSYISSSGSTIY |
| | | YADSVKGRFTISRDNAKNSLYLQMNSLRA |
| | | EDTAVYYCARDFYDSKIFDYWGQGTLVT |
| | | VSS |
| Heavy Chain | SEQ ID NO: 9 | ASTKGPSVFPLAPSSKSTSGGTAALGCLVK |
| Constant Domain 1 | | DYFPEPVTVSWNSGALTSGVHTFPAVLQS |
| of Fab Region | | SGLYSLSSVVTVPSSSLGTQTYICNVNHKP |
| | | SNTKVDKKV |
| Heavy Chain Hinge | SEQ ID NO: 10 | EPKSCDKT |
| region (partial) of | | |
| Fab Region | | |
| Heavy Chain Fab | SEQ ID NO: 11 | QVQLVESGGGLVKPGGSLRLSCAASGFTF |
| Region (full) | | SDYYMSWIRQAPGKGLEWVSYISSSGSTIY |
| | | YADSVKGRFTISRDNAKNSLYLQMNSLRA |
| | | EDTAVYYCARDFYDSKIFDYWGQGTLVT |
| | | VSSASTKGPSVFPLAPSSKSTSGGTAALGC |
| | | LVKDYFPEPVTVSWNSGALTSGVHTFPAV |
| | | LQSSGLYSLSSVVTVPSSSLGTQTYICNVN |
| | | HKPSNTKVDKKVEPKSCDKT |
| Heavy Chain | SEQ ID NO: 14 | QVQLVESGGGLVKPGGSLRLSCAASGFTF |
| (full) | | SDYYMSWIRQAPGKGLEWVSYISSSGSTIY |
| | | YADSVKGRFTISRDNAKNSLYLQMNSLRA |
| | | EDTAVYYCARDFYDSKIFDYWGQGTLVT |
| | | VSSASTKGPSVFPLAPSSKSTSGGTAALGC |
| | | LVKDYFPEPVTVSWNSGALTSGVHTFPAV |
| | | LQSSGLYSLSSVVTVPSSSLGTQTYICNVN |
| | | HKPSNTKVDKKVEPKSCDKTHTCPPCPAP |
| | | ELLGGPSVFLFPPKPKDTLMISRTPEVTCV |
| | | VVDVSHEDPEVKFNWYVDGVEVHNAKTK |
| | | PREEQYNSTYRVVSVLTVLHQDWLNGKE |
| | | YKCKVSNKALPAPIEKTISKAKGQPREPQV |
| | | YTLPPSRDELTKNQVSLTCLVKGFYPSDIA |
| | | VEWESNGQPENNYKTTPPVLDSDGSFFLY |
| | | SKLTVDKSRWQQGNVFSCSVMHEALHNH |
| | | YTQKSLSLSPGK |
| Light Chain CDR-1 | SEQ ID NO: 4 | RASQSISSYLN |
| Light Chain CDR-2 | SEQ ID NO: 5 | AASSLQS |
| Light Chain CDR-3 | SEQ ID NO: 6 | MEGLKTPFT |
| Light Chain Variable | SEQ ID NO: 8 | DIQMTQSPSSLSASVGDRVTITCRASQSISS |
| Region | | YLNWYQQKPGKAPKLLIYAASSLQSGVPS |
| | | RFSGSGSGTDFTLTISSLQPEDVGVYYCME |
| | | GLKTPFTFGPGTKLEIKR |
| Light Chain Constant | SEQ ID NO: 12 | TVAAPSVFIFPPSDEQLKSGTASVVCLLNN |
| Domain of Fab | | FYPREAKVQWKVDNALQSGNSQESVTEQ |
| | | DSKDSTYSLSSTLTLSKADYEKHKVYACE |
| | | VTHQGLSSPVTKSFNRGEC |
| Light Chain Fab | SEQ ID NO: 13 | DIQMTQSPSSLSASVGDRVTITCRASQSISS |
| Region (full) | | YLNWYQQKPGKAPKLLIYAASSLQSGVPS |
| | | RFSGSGSGTDFTLTISSLQPEDVGVYYCME |
| | | GLKTPFTFGPGTKLEIKRTVAAPSVFIFPPS |
| | | DEQLKSGTASVVCLLNNFYPREAKVQWK |
| | | VDNALQSGNSQESVTEQDSKDSTYSLSSTL |
| | | TLSKADYEKHKVYACEVTHQGLSSPVTKS |
| | | FNRGEC |
| Light Chain (full) | SEQ ID NO: 15 | DIQMTQSPSSLSASVGDRVTITCRASQSISS |
| | | YLNWYQQKPGKAPKLLIYAASSLQSGVPS |
| | | RFSGSGSGTDFTLTISSLQPEDVGVYYCME |
| | | GLKTPFTFGPGTKLEIKRTVAAPSVFIFPPS |
| | | DEQLKSGTASVVCLLNNFYPREAKVQWK |
| | | VDNALQSGNSQESVTEQDSKDSTYSLSSTL |
| | | TLSKADYEKHKVYACEVTHQGLSSPVTKS |
| | | FNRGEC |

In Table 1, each of these amino acid sequences is in an IgG format. However, one of skill in the art will recognize, as described elsewhere herein, that these sequences can be engineered to different immunoglobulin formats, and/or to produce antigen binding fragments, and/or otherwise engineered (for example by humanization), while retaining the key determinants for ADAM10—i.e., the CDRs.

The nucleotide sequences for the CDRs, heavy and light chain variable regions, and full heavy and light chains of 1H5 are provided in Table 2, which also provides SEQ ID NOS for each nucleotide sequence.

TABLE 2
Nucleotide sequences of 1H5 CDRs (according to Kabat numbering), heavy and
light chain variable regions, and full heavy and light chains.

| Region/Domain | SEQ ID NO | Nucleotide Sequence |
|---|---|---|
| Heavy Chain CDR-1 | SEQ ID NO: 16 | GATTACTATATGAGC |
| Heavy Chain CDR-2 | SEQ ID NO: 17 | TACATCTCAAGTTCCGGCTCTACAATCTA |
| | | CTATGCAGATTCCGTGAAAGGG |
| Heavy Chain CDR-3 | SEQ ID NO: 18 | GATTTCTATGATAGCAAGATTTTTGACTA |
| | | C |
| Heavy Chain | SEQ ID NO: 22 | CAAGTTCAATTAGTGGAGAGCGGCGGCG |
| Variable Region | | GATTGGTTAAACCGGGGGGCAGTCTGCG |
| | | TTTAAGCTGCGCAGCTTCCGGGTTTACTT |
| | | TTTCGGATTACTATATGAGCTGGATTCGT |
| | | CAGGCTCCCGGTAAAGGGTTGGAATGGG |
| | | TTAGCTACATCTCAAGTTCCGGCTCTACA |
| | | ATCTACTATGCAGATTCCGTGAAAGGGC |
| | | GCTTCACCATCTCTCGTGACAATGCAAA |
| | | GAACAGTTTATACTTACAAATGAACTCG |
| | | CTGCGTGCGGAAGATACGGCTGTGTATT |
| | | ACTGTGCGAGAGATTTCTATGATAGCAA |
| | | GATTTTTGACTACTGGGGCCAGGGAACC |
| | | CTGGTCACCGTCTCCTCA |
| Heavy Chain | SEQ ID NO: 24 | GCGTCGACCAAAGGGCCCAGTGTCTTTC |
| Constant Domain 1 | | CGTTAGCTCCGTCTTCCAAAAGTACGTC |
| of Fab Region | | GGGCGGCACGGCGGCACTTGGCTGCTTA |
| | | GTCAAGGACTACTTTCCCGAACCCGTTA |
| | | CTGTCTCTTGGAATAGTGGGGCTTTGACC |
| | | AGTGGGGTTCATACCTTTCCAGCCGTACT |
| | | TCAGTCTTCAGGGCTGTATAGTTTAAGTT |
| | | CAGTGGTTACAGTGCCCTCTTCATCTTTA |
| | | GGGACCCAAACCTATATCTGCAACGTCA |
| | | ATCATAAGCCTTCAAATACCAAGGTTGA |
| | | CAAAAAGGTG |
| Heavy Chain Hinge | SEQ ID NO: 25 | GAGCCCAAATCTTGTGACAAAACT |
| region (partial) of | | |
| Fab Region | | |
| Heavy Chain Fab | SEQ ID NO: 26 | CAAGTTCAATTAGTGGAGAGCGGCGGCG |
| Region (full) | | GATTGGTTAAACCGGGGGGCAGTCTGCG |
| | | TTTAAGCTGCGCAGCTTCCGGGTTTACTT |
| | | TTTCGGATTACTATATGAGCTGGATTCGT |
| | | CAGGCTCCCGGTAAAGGGTTGGAATGGG |
| | | TTAGCTACATCTCAAGTTCCGGCTCTACA |
| | | ATCTACTATGCAGATTCCGTGAAAGGGC |
| | | GCTTCACCATCTCTCGTGACAATGCAAA |
| | | GAACAGTTTATACTTACAAATGAACTCG |
| | | CTGCGTGCGGAAGATACGGCTGTGTATT |
| | | ACTGTGCGAGAGATTTCTATGATAGCAA |
| | | GATTTTTGACTACTGGGGCCAGGGAACC |
| | | CTGGTCACCGTCTCCTCAGCGTCGACCA |
| | | AAGGGCCCAGTGTCTTTCCGTTAGCTCC |
| | | GTCTTCCAAAAGTACGTCGGGCGGCACG |
| | | GCGGCACTTGGCTGCTTAGTCAAGGACT |
| | | ACTTTCCCGAACCCGTTACTGTCTCTTGG |
| | | AATAGTGGGGCTTTGACCAGTGGGGTTC |
| | | ATACCTTTCCAGCCGTACTTCAGTCTTCA |
| | | GGGCTGTATAGTTTAAGTTCAGTGGTTA |
| | | CAGTGCCCTCTTCATCTTTAGGGACCCA |
| | | AACCTATATCTGCAACGTCAATCATAAG |
| | | CCTTCAAATACCAAGGTTGACAAAAAGG |
| | | TGGAGCCCAAATCTTGTGACAAAACT |
| Heavy Chain | SEQ ID NO: 29 | CAGGTCCAGTTGGTTGAAAGTGGCGGCG |
| (full) | | GGCTCGTCAAACCAGGAGGCTCACTTCG |
| | | GCTGAGCTGTGCGGCGTCCGGTTTCACTT |
| | | TTAGTGATTACTATATGTCCTGGATACGG |
| | | CAAGCGCCTGGGAAAGGTTTGGAATGGG |
| | | TGTCATATATTTCATCAAGCGGATCAAC |
| | | AATCTACTATGCTGACTCCGTGAAAGGC |
| | | CGGTTTACAATCAGCAGAGACAATGCAA |
| | | AAAATAGCCTCTATCTCCAGATGAACTC |
| | | TCTGCGGGCAGAAGATACTGCAGTGTAT |
| | | TACTGCGCCCGAGATTTCTACGATAGTA |
| | | AGATTTTCGACTATTGGGGTCAGGGAAC |
| | | CTTGGTTACAGTTAGCTCAGCCTCCACC |
| | | AAGGGCCCAAGTGTCTTTCCGTTAGCTC |
| | | CGTCTTCCAAAAGTACGTCGGGCGGCAC |
| | | GGCGGCACTTGGCTGCTTAGTCAAGGAC |
| | | TACTTTCCCGAACCCGTTACTGTCTCTTG |
| | | GAATAGTGGGGCTTTGACCAGTGGGGTT |
| | | CATACCTTTCCAGCCGTACTTCAGTCTTC |
| | | AGGGCTGTATAGTTTAAGTTCAGTGGTT |
| | | ACAGTGCCCTCTTCATCTTTAGGGACCC |
| | | AAACCTATATCTGCAACGTCAATCATAA |
| | | GCCTTCAAATACCAAGGTTGACAAAAAG |
| | | GTGGAGCCCAAATCTTGTGACAAAACTC |
| | | ACACATGCCCACCGTGCCCAGCACCTGA |
| | | ACTCCTGGGGGGACCGTCAGTCTTCCTC |
| | | TTCCCCCCAAAACCCAAGGACACCCTCA |
| | | TGATCTCCCGGACCCCTGAGGTCACATG |
| | | CGTGGTGGTGGACGTGAGCCACGAAGAC |
| | | CCTGAGGTCAAGTTCAACTGGTACGTGG |
| | | ACGGCGTGGAGGTGCATAATGCCAAGAC |
| | | AAAGCCGCGGGAGGAGCAGTACAACAG |
| | | CACGTACCGTGTGGTCAGCGTCCTCACC |
| | | GTCCTGCACCAGGACTGGCTGAATGGCA |
| | | AGGAGTACAAGTGCAAGGTCTCCAACAA |
| | | AGCCCTCCCAGCCCCCATCGAGAAAACC |
| | | ATCTCCAAAGCCAAAGGGCAGCCCCGAG |
| | | AACCACAGGTGTACACCCTGCCCCCATC |
| | | CCGGGATGAGCTGACCAAGAACCAGGTC |
| | | AGCCTGACCTGCCTGGTCAAAGGCTTCT |
| | | ATCCCAGCGACATCGCCGTGGAGTGGGA |
| | | GAGCAATGGGCAGCCGGAGAACAACTA |
| | | CAAGACCACGCCTCCCGTGCTGGACTCC |
| | | GACGGCTCCTTCTTCCTCTACAGCAAGCT |
| | | CACCGTGGACAAGAGCAGGTGGCAGCA |
| | | GGGGAACGTCTTCTCATGCTCCGTGATG |
| | | CATGAGGCTCTGCACAACCACTACACGC |
| | | AGAAGAGCCTCTCCCTGTCTCCGGGTAA |
| | | A |
| Light Chain CDR-1 | SEQ ID NO: 19 | CGTGCATCCCAAAGCATTTCGAGTTACTT |
| | | GAAC |
| Light Chain CDR-2 | SEQ ID NO: 20 | GCTGCCAGTAGTCTTCAATCT |
| Light Chain CDR-3 | SEQ ID NO: 21 | ATGGAAGGTTTAAAAACTCCATTCACT |
| Light Chain Variable | SEQ ID NO: 23 | GATATTCAAATGACCCAGTCTCCTTCTTC |
| Region | | CTTGAGCGCGTCTGTGGGAGACCGTGTA |
| | | ACTATCACGTGTCGTGCATCCCAAAGCA |
| | | TTTCGAGTTACTTGAACTGGTATCAACA |
| | | AAAGCCTGGTAAAGCGCCCAAACTGTTG |
| | | ATCTATGCTGCCAGTAGTCTTCAATCTGG |
| | | TGTGCCCTCGCGCTTCTCAGGTTCGGGGT |
| | | CAGGAACGGACTTTACCCTGACCATTAG |
| | | CTCTCTGCAACCGGAGGATGTCGGAGTT |
| | | TATTACTGCATGGAAGGTTTAAAAACTC |
| | | CATTCACTTTCGGCCCTGGGACCAAACT |
| | | GGAGATCAAACGT |
| Light Chain Constant | SEQ ID NO: 27 | ACGGTGGCTGCTCCGTCGGTGTTTATCTT |
| Domain of Fab | | TCCACCGAGTGACGAACAACTGAAGTCG |
| Region | | GGGACTGCCAGCGTTGTTTGCCTTCTGA |
| | | ACAATTTTTACCCCCGCGAGGCGAAGGT |
| | | TCAGTGGAAAGTCGATAATGCGTTACAG |
| | | AGCGGGAATAGCCAGGAAAGTGTTACTG |
| | | AACAGGATAGTAAAGACAGCACTTACAG |
| | | CTTATCGTCTACACTTACTCTGAGTAAAG |
| | | CGGATTATGAAAAGCATAAGGTGTACGC |
| | | TTGCGAGGTTACACATCAAGGGTTATCT |
| | | TCGCCAGTCACGAAGAGCTTTAATCGCG |
| | | GCGAGTGC |
| Light Chain Fab | SEQ ID NO: 28 | GATATTCAAATGACCCAGTCTCCTTCTTC |
| Region (full) | | CTTGAGCGCGTCTGTGGGAGACCGTGTA |
| | | ACTATCACGTGTCGTGCATCCCAAAGCA |
| | | TTTCGAGTTACTTGAACTGGTATCAACA |
| | | AAAGCCTGGTAAAGCGCCCAAACTGTTG |
| | | ATCTATGCTGCCAGTAGTCTTCAATCTGG |
| | | TGTGCCCTCGCGCTTCTCAGGTTCGGGGT |
| | | CAGGAACGGACTTTACCCTGACCATTAG |
| | | CTCTCTGCAACCGGAGGATGTCGGAGTT |
| | | TATTACTGCATGGAAGGTTTAAAAACTC |
| | | CATTCACTTTCGGCCCTGGGACCAAACT |
| | | GGAGATCAAACGTACGGTGGCTGCTCCG |
| | | TCGGTGTTTATCTTTCCACCGAGTGACGA |
| | | ACAACTGAAGTCGGGGACTGCCAGCGTT |
| | | GTTTGCCTTCTGAACAATTTTTACCCCCG |
| | | CGAGGCGAAGGTTCAGTGGAAAGTCGAT |
| | | AATGCGTTACAGAGCGGGAATAGCCAGG |
| | | AAAGTGTTACTGAACAGGATAGTAAAGA |
| | | CAGCACTTACAGCTTATCGTCTACACTTA |
| | | CTCTGAGTAAAGCGGATTATGAAAAGCA |
| | | TAAGGTGTACGCTTGCGAGGTTACACAT |
| | | CAAGGGTTATCTTCGCCAGTCACGAAGA |
| | | GCTTTAATCGCGGCGAGTGC |
| Light Chain | SEQ ID NO: 30 | GATATACAAATGACGCAGTCTCCTAGCA |
| (full) | | GTCTCTCCGCATCCGTGGGGGACCGCGT |
| | | AACAATCACTTGTCGCGCCAGCCAGTCA |
| | | ATTTCCTCCTACCTTAATTGGTATCAACA |
| | | GAAACCGGGAAAAGCCCCGAAACTCCT |
| | | GATTTATGCAGCTTCATCACTCCAGAGT |
| | | GGCGTACCGTCAAGGTTTAGTGGTTCTG |
| | | GTTCTGGGACTGATTTCACGCTGACGAT |
| | | CAGCTCTTTGCAACCGGAGGATGTAGGG |
| | | GTATATTATTGCATGGAAGGATTGAAAA |
| | | CGCCATTTACCTTCGGACCGGGGACAAA |
| | | ACTGGAAATTAAACGTACGGTCGCTGCG |
| | | CCCAGTGTATTTATCTTTCCTCCCAGCGA |
| | | TGAGCAACTCAAAAGTGGAACCGCCAGT |
| | | GTGGTGTGTTTGTTGAATAACTTTTATCC |
| | | CAGAGAAGCTAAGGTACAATGGAAGGT |
| | | CGATAACGCCCTTCAGTCTGGAAATAGT |
| | | CAGGAAAGTGTCACAGAGCAGGATTCCA |
| | | AGGACTCCACGTATAGTCTTAGTAGTAC |
| | | CTTGACTCTGAGCAAAGCAGACTATGAG |
| | | AAGCACAAGGTGTATGCTTGTGAAGTGA |
| | | CGCACCAGGGCCTCTCCTCCCCCGTGAC |
| | | GAAGAGCTTTAATCGCGGAGAGTGC |

A summary of the sequences provided herein are presented in Table 3.

TABLE 3
1H5 antibody sequence summary.

| Sequence Summary | SEQ ID NO. |
|---|---|
| Amino acid sequence of heavy chain CDR-1 | SEQ ID NO. 1 |
| Amino acid sequence of heavy chain CDR-2 | SEQ ID NO. 2 |
| Amino acid sequence of heavy chain CDR-3 | SEQ ID NO. 3 |
| Amino acid sequence of heavy chain CDR-1 | SEQ ID NO. 4 |
| Amino acid sequence of heavy chain CDR-2 | SEQ ID NO. 5 |
| Amino acid sequence of heavy chain CDR-3 | SEQ ID NO. 6 |
| Amino acid sequence of heavy chain variable region | SEQ ID NO. 7 |
| Amino acid sequence of light chain variable region | SEQ ID NO. 8 |
| Amino acid sequence of heavy chain constant domain | SEQ ID NO. 9 |
| 1 of Fab region | |
| Amino acid sequence of heavy chain hinge region | SEQ ID NO. 10 |
| (partial) of Fab region | |
| Amino acid sequence of heavy chain Fab region (full) | SEQ ID NO. 11 |
| Amino acid sequence of light chain constant domain of | SEQ ID NO. 12 |
| Fab region | |
| Amino acid sequence of light chain Fab region (full) | SEQ ID NO. 13 |
| Amino acid sequence of heavy chain (full) | SEQ ID NO. 14 |
| Amino acid sequence of light chain (full) | SEQ ID NO. 15 |
| Nucleotide sequence of heavy chain CDR-1 | SEQ ID NO. 16 |
| Nucleotide sequence of heavy chain CDR-2 | SEQ ID NO. 17 |
| Nucleotide sequence of heavy chain CDR-3 | SEQ ID NO. 18 |
| Nucleotide sequence of light chain CDR-1 | SEQ ID NO. 19 |
| Nucleotide sequence of light chain CDR-2 | SEQ ID NO. 20 |
| Nucleotide sequence of light chain CDR-3 | SEQ ID NO. 21 |
| Nucleotide sequence of heavy chain variable region | SEQ ID NO. 22 |
| Nucleotide sequence of light chain variable region | SEQ ID NO. 23 |
| Nucleotide sequence of heavy chain constant domain | SEQ ID NO. 24 |
| 1 of Fab region | |
| Nucleotide sequence of heavy chain hinge region | SEQ ID NO. 25 |
| (partial) of Fab region | |
| Nucleotide sequence of heavy chain Fab region (full) | SEQ ID NO. 26 |
| Nucleotide sequence of light chain constant domain of | SEQ ID NO. 27 |
| Fab region | |
| Nucleotide sequence of light chain Fab region (full) | SEQ ID NO. 28 |
| Nucleotide sequence of heavy chain (full) | SEQ ID NO. 29 |
| Nucleotide sequence of light chain (full) | SEQ ID NO. 30 |

In addition to providing the specific ADAM10 antibodies, and fragments thereof, whose sequences are provided in Table 1 and Table 2 above, the present invention also encompasses variants and equivalents of these ADAM10 antibodies and antibody fragments. For example, such variants include humanized, chimeric, optimized, germlined, and/or other versions of any of the anti-ADAM10 antibodies, or fragments thereof, disclosed herein. Likewise, in some embodiments variants of the specific sequences disclosed herein that comprise one or more substitutions, additions, deletions, or other mutations may be used. A heavy chain variable region and/or light chain variable region amino acid sequence or portion thereof, including a CDR sequence, can be, e.g., 85%, 90%, 95%, 96%, 97%, 98% or 99% similar to a sequence set forth herein, and/or comprise 1, 2, 3, 4, 5 or more substitutions, e.g., conservative substitutions, relative to a sequence set forth herein. In some embodiments an ADAM10 binding molecule according to the present invention comprises a heavy chain variable region and/or light chain variable region amino acid sequence, or portion thereof, that is 85%, 90%, 95%, 96%, 97%, 98% or 99% similar to that present in the specific sequences provided herein (e.g., SEQ ID NO. 7 and/or SEQ ID NO. 8) set forth herein, and/or comprise 1, 2, 3, 4, 5 or more substitutions. e.g., conservative substitutions, relative to that sequence, but comprises the specific CDR sequences found within such heavy chain and/or light chain variable regions—i.e., any mutations (such as substitutions, additions, deletions, etc.) are outside of the CDRs Such ADAM10 binding molecules, i.e., having heavy chain and light chain variable regions with a certain percent similarity to a heavy chain variable region or light chain variable region, or having one or more substitutions, e.g., conservative substitutions, can be obtained by mutagenesis (e.g., site-directed or PCR-mediated mutagenesis) of nucleic acid molecules encoding heavy chain and/or variable light chain variable regions described herein, followed by testing of the encoded altered binding molecule for binding to ADAM10, and optionally testing for retained function, such as: (a) inhibition of binding of ADAM10 to ADAM10 substrates (such as Notch, ephrin, and EGFR ligands), (b) inhibition of ADAM10-dependent proteolytic cleavage of ADAM10 substrates (such as Notch, ephrin, and EGFR ligands), (c) inhibition of activation (e.g., transactivation) of ADAM10 substrates (such as Notch, ephrin, and EGFR ligands), (d) inhibition of signaling by ADAM10 substrates, and/or (e) inhibition of proliferation of tumor cells in vitro or in vivo, for example using the functional assays described herein.

Subsequent sections of this patent disclosure provide further details regarding different variants of the specific ADAM10 binding molecules described herein that are within the scope of the present invention, and how to make and use such variants.

In some embodiments, the ADAM10 binding molecule is a humanized antibody, a chimeric antibody, a monoclonal antibody, a polyclonal antibody, a recombinant antibody, a bi-specific antibody, a multispecific antibody, or any combination thereof. In some embodiments, ADAM10 binding molecules comprise a Fab, a Fab′, a F(ab′)2, a Fd, a Fv, a scFv, a disulfide linked Fv, a V-NAR domain, an IgNar, an intrabody, an IgGΔCH2, a minibody, a F(ab′)₃, a tetrabody, a triabody, a diabody, a single-domain antibody, DVD-Ig, Fcab. mAb², a (scFv)₂, or a scFv-Fc.

An ADAM10 binding molecule provided herein can include, in addition to a heavy chain variable region and a light chain variable region, a heavy chain constant region or fragment thereof. In certain embodiments the heavy chain constant region is a human heavy chain constant region, e.g., a human IgG constant region, e.g., a human IgG1 constant region.

In certain embodiments, binding molecules of the invention are produced to comprise an altered Fc region, in which one or more alterations have been made in the Fc region in order to change functional and/or pharmacokinetic properties of the binding molecule. Such alterations may result in altered effector function, reduced immunogenicity, and/or an increased serum half-life. The Fc region interacts with a number of ligands, including Fc receptors, the complement protein Clq, and other molecules, such as proteins A and G These interactions are essential for a variety of effector functions and downstream signaling events including antibody dependent cell-mediated cytotoxicity (ADCC) and complement dependent cytotoxicity (CDC). Accordingly, in certain embodiments the ADAM10 binding molecules of the invention have reduced or ablated affinity for an Fc ligand responsible for facilitating effector function, compared to an ADAM10 binding molecule not comprising the modification in the Fc region. In particular embodiments, the ADAM10 binding molecule has no ADCC activity and/or no CDC activity. In certain embodiments, the ADAM10 binding molecule does not bind to an Fc receptor and/or complement factors. In certain embodiments, the ADAM10 binding molecule has no effector function. Selecting particular constant domains to optimize desired effector functions is within the ordinary skill in the art. In some embodiments, the binding molecule is of the IgG1 subtype, and optionally comprises the TM format (L234F/L235E/P331S), as disclosed above in the Definitions section.

In some embodiments, a heavy chain constant region or fragment thereof can include one or more amino acid substitutions relative to a wild-type IgG constant domain, wherein the modified IgG has an increased half-life compared to the half-life of an IgG having the wild-type IgG constant domain. For example, the IgG constant domain can contain one or more amino acid substitutions of amino acid residues at positions 251-257, 285-290, 308-314, 385-389, and 428-436, wherein the amino acid position numbering is according to the EU index as set forth in Kabat. In certain embodiments the IgG constant domain can contain one or more of a substitution of the amino acid at Kabat position 252 with Tyrosine (Y), Phenylalanine (F), Tryptophan (W), or Threonine (T), a substitution of the amino acid at Kabat position 254 with Threonine (T), a substitution of the amino acid at Kabat position 256 with Serine (S), Arginine (R), Glutamine (Q), Glutamic acid (E), Aspartic acid (D), or Threonine (T), a substitution of the amino acid at Kabat position 257 with Leucine (L), a substitution of the amino acid at Kabat position 309 with Proline (P), a substitution of the amino acid at Kabat position 311 with Serine (S), a substitution of the amino acid at Kabat position 428 with Threonine (T), Leucine (L), Phenylalanine (F), or Serine (S), a substitution of the amino acid at Kabat position 433 with Arginine (R), Serine (S), Isoleucine (I), Proline (P), or Glutamine (Q), or a substitution of the amino acid at Kabat position 434 with Tryptophan (W), Methionine (M), Seine (S), Histidine (H), Phenylalanine (F), or Tyrosine. More specifically, the IgG constant domain can contain amino acid substitutions relative to a wild-type human IgG constant domain including as substitution of the amino acid at Kabat position 252 with Tyrosine (Y), a substitution of the amino acid at Kabat position 254 with Threonine (T), and a substitution of the amino acid at Kabat position 256 with Glutamic acid (E). In some embodiments, the binding molecule is of the IgG1 subtype, and optionally comprises the triple mutant YTE, as disclosed supra in the Definitions section.

An ADAM10 binding molecule provided herein can include a light chain constant region or fragment thereof. In certain embodiments the light chain constant region is a kappa constant region or a lambda constant region. e.g., a human kappa constant region or a human lambda constant region.

In some embodiments, this disclosure provides ADAM10 binding molecules that can specifically bind to the same ADAM10 epitope as a binding molecule comprising the heavy chain variable region and light chain variable region of 1H5. The term “epitope” refers to a target protein determinant capable of binding to a binding molecule of the invention.

Epitopes usually consist of chemically active surface groupings of molecules such as amino acids or sugar side chains, and usually have specific three-dimensional structural characteristics, as well as specific charge characteristics. Conformational and non-conformational epitopes are distinguished in that the binding to the former but not the latter is lost in the presence of denaturing solvents. Such binding molecules can be identified based on their ability to cross-compete (e.g., to competitively inhibit the binding of, in a statistically significant manner) with binding molecules, such as 1H5, in standard ADAM10 binding or activity assays.

Accordingly, in one embodiment, the invention provides ADAM10 binding molecules that compete for binding to ADAM10 with another ADAM10 binding molecule of the invention, such as 1H5. The ability of a binding molecule to inhibit the binding of, e.g., 1H5, demonstrates that the test binding molecule can compete with 1H5 for binding to ADAM10, such a binding molecule can, according to non-limiting theory, bind to the same or a related (e.g., a structurally similar or spatially proximal) epitope on ADAM10 as the ADAM10 binding molecule with which it competes. In one embodiment, an anti-ADAM10 antibody or antigen-binding fragment thereof binds to the same epitope on ADAM10 as 1H5. The term “competes” indicates that a binding molecule competes unidirectionally for binding to ADAM10 with 1H5. The term “cross-competes” indicates that a binding molecule competes bidirectionally for binding to ADAM10 with 1H5.

ADAM10 binding molecules provided herein can have beneficial properties. For example, the binding molecule can inhibit, suppress, or block various ADAM10-mediated activities. e.g., proteolytic cleavage of cell surface EGFR molecules, and the associated transactivation thereof, which can be measured by assays known in the art.

In some embodiments, the binding molecules provided herein can bind to ADAM10 with a binding affinity characterized by a dissociation constant (K_{D}) of about 100 pM to about 0.5 nM as measured by a Biacore™ assay or on a Kinetic Exclusion Assay (KinExA) 3000 platform or on an Octet® instrument.

In certain embodiments, an anti-ADAM10 antibody or antigen-binding fragment thereof can specifically bind to ADAM10, e.g., human ADAM10, or an antigenic fragment thereof, with a dissociation constant or K_{D} of less than 10⁻⁶ M, or of less than 10⁻⁷ M, or of less than 10⁻⁸ M, or of less than 10⁻⁹ M as measured, e.g., by Biacore™ or KinExA® or Octet®.

The disclosure further provides an ADAM10 binding molecule that is conjugated to a heterologous agent, such as an antibody-drug conjugate. In certain embodiments, the agent can be an antimicrobial agent, a therapeutic agent, a prodrug, a peptide, a protein, an enzyme, a lipid, a biological response modifier, a pharmaceutical agent, a lymphokine, a heterologous antibody or fragment thereof, a detectable label, a polyethylene glycol (PEG), or a combination of two or more of any said agents.

In addition, the disclosure provides CAR T cells, in which the antigen recognition domain of the chimeric antigen receptor is derived from one or more portions of an ADAM10 binding molecule defined herein. For instance, the antigen recognition domain may comprise a heavy chain variable region that comprises a CDR-1 domain comprising or having an amino acid sequence of SEQ ID NO. 1, a CDR-2 domain comprising or having an amino acid sequence of SEQ ID NO. 2, and a CDR-3 domain comprising or having an amino acid sequence of SEQ ID NO. 3, and a light chain variable region that comprises a CDR-1 domain comprising or having an amino acid sequence of SEQ ID NO. 4, a CDR-2 domain comprising or having an amino acid sequence of SEQ ID NO. 5, and a CDR-3 domain comprising or having, an amino acid sequence of SEQ ID NO. 6. In some embodiments, the antigen recognition domain may comprise a heavy chain variable region comprising an amino acid sequence at least 95% identical to SEQ ID NO. 22, and a light chain variable region comprising an amino acid sequence at least 95% identical to SEQ ID NO. 23. In certain embodiments, the antigen recognition domain may comprise a heavy chain variable region comprising an amino acid sequence of SEQ ID NO. 22, and a light chain variable region comprising an amino acid sequence of SEQ ID NO. 23.

In some embodiments, the disclosure provides a composition, e.g., a pharmaceutical composition, comprising an ADAM10 binding molecule of the invention, optionally further comprising one or more carriers, diluents, excipients, or other additives.

### Preparation of ADAM10 Binding Molecules

Monoclonal anti-ADAM10 antibodies can be prepared using hybridoma methods, such as those described by Kohler & Milstein (1975). Using the hybridoma method, a mouse, hamster, or other appropriate host animal, is immunized to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol (PEG), to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g., RIA or ELISA) can then be propagated either in in vitro culture using standard methods (Goding, 1986) or in vivo as ascites tumors in an animal. The monoclonal antibodies can then be purified from the culture medium or ascites fluid.

ADAM10 binding molecules can also be made using recombinant DNA methods, for example, as described in U.S. Pat. No. 4,816,567. In some instances, the polynucleotides encoding a monoclonal antibody are isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains or antigen-binding fragments thereof are then cloned into suitable expression vectors, which when transfected into host cells such as E. coli cells, simian COS cells, Chinese hamster ovary (CHO) cells, or myeloma cells that do not otherwise produce immunoglobulin protein, binding molecules are generated by the host cells. Also, recombinant ADAM10 binding molecules can be isolated from phage display libraries expressing CDRs of the desired species, as described by McCafferty et al. (1990). Clackson et al. (1991), and Marks et al. (1991). Production and expression of nucleic acids comprising nucleotide sequences encoding ADAM110 binding molecules are discussed in more detail in the next section.

The polynucleotide(s) encoding a binding molecule can further be modified in a number of different manners using recombinant DNA technology to generate alternative binding molecules. In some embodiments, the constant domains of the light and heavy chains of, for example, a mouse monoclonal antibody can be substituted (1) for those regions of, for example, a human antibody to generate a chimeric antibody or (2) for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

In certain embodiments, the ADAM10 binding molecule is a human antibody or antigen-binding fragment thereof. Human antibodies can be directly prepared using various techniques known in the art. Immortalized human B lymphocytes immunized in vitro or isolated from an immunized individual that produce an antibody directed against a target antigen can be generated (See. e.g., Cole et al., 1985; Boemer et al., 1991; and U.S. Pat. No. 5,750,373).

The ADAM10 binding molecule can be selected from a phage library, where the phage library expresses human antibodies, as described, for example, by Vaughan et al. (1996), Sheets et al. (1998), and Marks et al. (1991). Techniques for the generation and use of antibody phage libraries are also described in U.S. Pat. Nos. 5,969,108, 6,172,197, 5,885,793, 6,521,404, 6,544,731; 6,555,313; 6,582,915; 6,593,081; 6,300,064; 6,653,068; 6,706,484; and 7,264,963; and in Rothe et al. (2007). An additional method is described herein in Example 1.

Affinity maturation strategies and chain shuffling strategies are known in the art and can be employed to generate high affinity human antibodies or antigen-binding fragments thereof. (See Marks et al., 1992).

In some embodiments, an ADAM 10 binding molecule can be a humanized antibody or antigen-binding fragment thereof. Methods for enginemen, humanizing, or resurfacing non-human or human antibodies can also be used and are well known in the art. A humanized, resurfaced, or similarly engineered antibody can have one or more amino acid residues from a source that is non-human. e.g., mouse, rat, rabbit, non-human primate, or other mammal. These non-human amino acid residues are replaced by residues that are often referred to as “import” residues, which are typically taken from an “import” variable, constant, or other domain of a known human sequence. Such imported sequences can be used to reduce immunogenicity or reduce, enhance, or modify binding, affinity, on-rate, off-rate, avidity, specificity, half-life, or any other suitable characteristic, as known in the art. In general, the CDR residues are directly and most substantially involved in influencing ADAM10 binding. Accordingly, part or all of the non-human or human CDR sequences are maintained while the non-human sequences of the variable and constant regions can be replaced with human or other amino acids. Humanization, resurfacing, or engineering of ADAM10 antibodies or antigen-binding fragments thereof can be performed using any known method, such as, but not limited to, those described in, Jones et al. (1986); Riechmann et al. (1988); Verhoeyen et al. (1988); Sims et al. (1993); Chothia & Lesk (1987); Carter et al. (1992); Presta et al. (1993); U.S. Pat. Nos. 5,639,641, 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567, 7,557,189; 7,538,195; and 7,342,110; International Application Nos. PCT/US98/16280: PCT/US96/18978; PCT/US91/09630; PCT/US91/05939; PCT/US94/01234; PCT/GB89/01334; PCT/GB91/01134; PCT/GB92/01755; International Patent Application Publication Nos. WO90/14443; WO90/14424; WO90/14430; and European Patent Publication No. EP 229246.

Anti-ADAM10 humanized antibodies and antigen-binding fragments thereof can also be made in transgenic mice containing human immunoglobulin loci that are capable, upon immunization, of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In certain embodiments the ADAM10 binding molecule is an anti-ADAM10 antibody fragment. Various techniques are known for the production of antibody fragments. Traditionally, these fragments are derived via proteolytic digestion of intact antibodies (see. e.g., Morimoto & Inouye (1993); Brennan et al. (1985)). In certain embodiments, anti-ADAM10 antibody fragments are produced recombinantly. Fab, Fv, and scFv antibody fragments can all be expressed in and secreted from E co/i or other host cells, thus allowing the production of large amounts of these fragments. Such anti-ADAM10 antibody fragments can also be isolated from the antibody phage libraries discussed above. Anti-ADAM10 antibody fragments can also be linear antibodies, as described in U.S. Pat. No. 5,641,870. Other techniques for the production of antibody fragments will be apparent to the skilled practitioner.

According to the present invention, techniques can be adapted for the production of single-chain antibodies specific to ADAM10 (see. e.g., U.S. Pat. No. 4,946,778). In addition, methods can be adapted for the construction of Fab expression libraries (see. e.g., Huse et al., 1989) to allow rapid and effective identification of monoclonal Fab fragments with the desired specificity for ADAM10. Antibody fragments can also be produced by techniques in the art including, but not limited to: (a) a F(ab′)2 fragment produced by pepsin digestion of an antibody molecule; (b) a Fab fragment generated by reducing the disulfide bridges of an F(ab′)2 fragment, (c) a Fab fragment generated by the treatment of the antibody molecule with papain and a reducing agent, and (d) Fv fragments.

In some embodiments, the ADAM10 binding molecules can be modified in order to reduce or eliminate effector function. This can be achieved, for example, by the triple mutation (TM) L234F/L235E/P331S in the Fc domain of IgG1. Other mutations that reduce effector function are known in the art (see. e.g., Armour et al, 1999; Shields et al., 2001).

In certain embodiments, an ADAM10 binding molecule can be modified to increase its serum half-life. This can be achieved, for example, by incorporation of a salvage receptor binding epitope into the binding molecule by mutation of the appropriate region, or by incorporating the epitope into a peptide tag that is then fused to the binding molecule at either end or in the middle (e.g., by DNA or peptide synthesis), or by YTE mutation. Other methods to increase the serum half-life of an antibody or antigen-binding fragment thereof, e.g., conjugation to a heterologous molecule such as PEG, are known in the art.

Heteroconjugate ADAM10 antibodies and antigen-binding fragments thereof are also within the scope of the present invention. Heteroconjugate antibodies are composed of two covalently joined antibodies. Such antibodies have, for example, been proposed to target immune cells to unwanted cells (see, e.g., U.S. Pat. No. 4,676,980). It is contemplated that heteroconjugate anti-ADAM10 antibodies and antigen-binding fragments thereof can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

An ADAM10 binding molecule can be modified to contain additional chemical moieties not normally part of the protein. Such moieties can improve the characteristics of the binding molecule, for example, solubility, biological half-life, or absorption. The moieties can also reduce or eliminate any undesirable side effects of the binding molecule. An overview of those moieties can be found in Remington's Pharmaceutical Sciences (2000).

### Polynucleotides Encoding ADAM10 Binding Molecules, Preparation and Expression Thereof

This disclosure provides certain polynucleotides comprising nucleic acid sequences that encode ADAM10 binding molecules. The polynucleotides of the invention can be in the form of RNA or in the form of DNA. DNA includes cDNA, genomic DNA, and synthetic DNA; and can be double-stranded or single-stranded, and, if single stranded, can be the coding strand or non-coding (anti-sense) strand.

In certain embodiments, the polynucleotide can be isolated. In certain embodiments, the polynucleotide can be substantially pure. In certain embodiments, the polynucleotide can be cDNA or are derived from cDNA. In certain embodiments, the polynucleotide can be recombinantly produced. In certain embodiments, the polynucleotide can comprise the coding sequence for a mature polypeptide, fused in the same reading frame to a polynucleotide which aids, for example, in expression and optionally, secretion, of a polypeptide from a host cell (e.g., a promoter or other regulatory sequence, a leader sequence that functions as a secretory sequence for controlling transport of a polypeptide from the cell). The polypeptide having a leader sequence is a pre-protein and can have the leader sequence cleaved by the host cell to form the mature form of the polypeptide. The polynucleotide can also encode an ADAM10 binding pro-protein which is the mature protein plus additional 5′ amino acid residues.

The disclosure provides an isolated polynucleotide comprising a nucleic acid encoding an ADAM10 binding molecule comprising an amino acid sequence from a heavy chain and/or light chain variable region having 85%, 90%, 95%, 96%, 97%, 98% or 99% similarity to an amino acid sequence set forth herein, and/or comprising 1, 2, 3, 4, 5 or more amino acid substitutions. e.g., conservative substitutions, relative to an amino acid sequence set forth herein, such as a sequence from 1H5.

The disclosure provides an isolated polynucleotide comprising a nucleic acid comprising a nucleotide sequence from a heavy chain and/or light chain variable region having 85%, 90%, 95%, 96%, 97%, 98% or 99% similarity to a nucleotide sequence set forth herein, and/or comprising 1, 2, 3, 4, 5 or more nucleotide substitutions, relative to a nucleotide sequence set forth herein, such as a sequence from 1H5 (see Table 2).

In addition, the disclosure provides an isolated polynucleotide comprising a nucleic acid having a nucleotide sequence from a heavy chain CDR-1, CDR-2, and CDR-3 having nucleotide sequences of SEQ ID NOs. 16, 17, and 18, respectively; and having a nucleotide sequence from a light chain CDR-1, CDR-2, and CDR-3 having nucleotide sequences of SEQ ID NOs. 19, 20, and 21, respectively.

Further, the disclosure provides an isolated polynucleotide comprising a nucleic acid having a nucleotide sequence from a heavy chain having nucleotide sequence of SEQ ID NO. 29, and having a nucleotide sequence from a light chain having nucleotide sequence of SEQ ID NO. 30.

In certain embodiments the polynucleotide that comprises the coding sequence for the ADAM10 binding molecule is fused in the same reading frame as a marker sequence that allows, for example, for purification of the encoded polypeptide. For example, the marker sequence can be a hexa-histidine tag (HHHHHH (SEQ ID NO: 31)) supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used.

Polynucleotide variants are also provided. Polynucleotide variants can contain alterations in the coding regions, non-coding regions, or both. In some embodiments, polynucleotide variants contain alterations that produce silent substitutions, additions, or deletions, but do not alter the properties or activities of the encoded polypeptide. In some embodiments, polynucleotide variants are produced by silent substitutions due to the degeneracy of the genetic code. Polynucleotide variants can be produced for a variety of reasons, e.g., to optimize codon expression for a particular host (change codons in the human mRNA to those preferred by a bacterial host such as E. coli).

The invention includes vectors comprising the polynucleotides described above. Suitable vectors are described elsewhere herein, and are known to those of ordinary skill in the art. In some embodiments, a polynucleotide comprising a nucleic acid encoding a heavy chain variable region or portion thereof, and a polynucleotide comprising a nucleic acid encoding a light chain variable region or portion thereof, can reside in a single vector, or can be on separate vectors. In some embodiments, polynucleotides comprising nucleic acids encoding heavy and light chain CDR-1, CDR-2, and CDR-3, or portions thereof, can reside in a single vector, or can be on separate vectors. In some embodiments, polynucleotides comprising nucleic acids encoding a heavy chain and a light chain, or portions thereof, can reside in a single vector, or can be on separate vectors. Accordingly, the disclosure provides one or more vectors comprising the polynucleotides described above.

In certain embodiments, the disclosure provides a composition, e.g., a pharmaceutical composition, comprising a polynucleotide or vector as described above, optionally further comprising one or more carriers, diluents, excipients, or other additives.

The disclosure further provides a host cell comprising a polynucleotide or vector of the invention, wherein the host cell can, in some instances, express a binding molecule that specifically binds to ADAM10. Such a host cell can be utilized in a method of making an ADAM10 binding molecule, where the method includes (a) culturing the host cell and (b) isolating the binding molecule from the host cell or from the culture medium, if the binding molecule is secreted by the host cell.

In some embodiments a nucleotide sequence encoding an ADAM10 binding molecule can be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest. For example, a complete amino acid sequence can be used to construct a back-translated gene. Further, a nucleotide oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed, e.g., by nucleotide sequencing, restriction mapping, and/or expression of a biologically active polypeptide in a suitable host. In order to obtain high expression levels of a transfected gene in a host, the gene can be operatively linked to or associated with transcriptional and translational expression control sequences that are functional in the chosen expression host.

In certain embodiments, recombinant expression vectors are used to amplify and express DNA encoding ADAM10 binding molecules. Recombinant expression vectors are replicable DNA constructs that have synthetic or cDNA-derived DNA fragments encoding a polypeptide chain of an ADAM10 binding molecule, operatively linked to suitable transcriptional or translational regulatory elements derived from mammalian, microbial, viral or insect genes A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail below. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide, a promoter is operatively linked to a coding sequence if it controls the transcription of the sequence; or a ribosome binding site is operatively linked to a coding sequence if it is positioned so as to permit translation. Structural elements intended for use in yeast expression systems include a leader sequence enabling extracellular secretion of translated protein by a host cell. Alternatively, where a recombinant protein is expressed without a leader or transport sequence, the protein can include an N-terminal methionine residue. This residue can optionally be subsequently cleaved from the expressed recombinant protein to provide a final product.

The choice of expression control sequence and expression vector will depend upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR 1, pBR322, pMB9 and their derivatives, wider host range plasmids, such as M13, and filamentous single-stranded DNA phages.

Suitable host cells for expression of an ADAM10 binding molecule include prokaryotes, yeast, insect, or higher eukaryotic cells under the control of appropriate promoters. Prokaryotes include gram negative or gram-positive organisms, for example E coli or bacilli. Higher eukaryotic cells include established cell lines of mammalian origin as described below. Cell-free translation systems could also be employed. Additional information regarding methods of protein production, including antibody production, can be found in, e.g., U.S. Patent Publication No. 200810187954, U.S. Pat. Nos. 6,413,746 and 6,660,501, and International Patent Publication No. WO 04009823.

Various mammalian or insect cell culture systems can be advantageously employed to express recombinant ADAM10 binding molecules. Expression of recombinant proteins in mammalian cells can be performed because such proteins are generally correctly folded, appropriately modified, and completely functional. Examples of suitable mammalian host cell lines include 293 cells (e.g., HEK-293, HEK-293T, AD293), the COS-7 lines of monkey kidney cells described by Gluzman (1981), and other cell lines including, for example, L cells. C127, 313. Chinese hamster ovary (CHO). HeLa, and BHK cell lines. Mammalian expression vectors can comprise non-transcribed elements, such as an origin of replication, a suitable promoter and enhancer linked to the gene to be expressed, and other 5′ or 3′ flanking non-transcribed sequences, and 5′ or 3 non-translated sequences, such as necessary ribosome binding sites, a polyadenylation site, splice donor and acceptor sites, and transcriptional termination sequences. Baculovirus systems for production of heterologous proteins in insect cells are reviewed by Luckow and Summers (1988).

ADAM10 binding molecules produced by a transformed host can be purified according to any suitable method. Such standard methods include chromatography (e.g., ion exchange, affinity, and sizing column chromatography), centrifugation, differential solubility, or by any other standard technique for protein purification. Affinity tags such as hexahistidine, maltose binding domain, influenza coat sequence, and glutathione-S-transferase can be attached to the protein to allow easy purification by passage over an appropriate affinity column. Isolated proteins can also be physically characterized using such techniques as proteolysis, nuclear magnetic resonance and x-ray crystallography.

For example, supernatants from systems that secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose, or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify an ADAM10 binding molecule. Some or all of the foregoing purification steps, in various combinations, can also be employed to provide a homogeneous recombinant protein.

A recombinant ADAM10 binding molecule produced in bacterial culture can be isolated, for example, by initial extraction from cell pellets, followed by one or more concentration, salting-out, aqueous ion exchange, or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U S Patent Publication Nos. 2008/0312425, 2008/0177048, and 2009/0187005.

### Use of ADAM10 Binding Molecules

The present invention provides various methods of using the ADAM10 binding molecules described herein. Such methods include, but are not limited to, use of the ADAM10 binding molecules described herein for: (a) inhibition of binding of ADAM10 to ADAM10 substrates (such as Notch, ephrin, and EGFR ligands), (b) inhibition of ADAM10-dependent proteolytic cleavage of ADAM10 substrates (such as Notch, ephrin, and EGFR ligands), (c) inhibition of activation (e.g., transactivation) of ADAM10 substrates (such as Notch, ephrin, and EGFR ligands), (d) inhibition of signaling by ADAM10 substrates, and (e) inhibition of proliferation of tumor cells in vitro or in vivo, such as tumor cells whose proliferation is driven, at least in part, by ADAM10-dependent proteolytic cleavage of ADAM10 substrates (such as Notch, ephrin, and EGFR ligands). Such tumor cells include, but are not limited to, colon, breast, ovarian, glioma (including glioblastoma), and lung adenocarcinoma (non-small cell lung cancer or NSCLC).

In some embodiments, the ADAM10 binding molecules provided herein are useful for the treatment of, and/or prevention of recurrence of, cancer. Examples of cancers that may be treated, or the recurrence of which may be prevented, using the ADAM10 binding molecules of the invention include colon, breast, ovarian, glioma, and lung cancers. For example, in one embodiment, the present invention provides a method of treatment, the method comprising administering to a subject in need thereof an ADAM10 binding molecule, or a composition comprising an ADAM10 binding molecule, such as, for example, a pharmaceutical composition. In some such embodiments, the subject has colorectal cancer, colon cancer, breast cancer, ovarian cancer, lung cancer, non-small cell lung cancer, brain cancer, glioma, glioblastoma, or neuroblastoma. In certain such embodiments, the subject has colon cancer, breast cancer, ovarian cancer, a glioma, or lung cancer.

Similarly, the ADAM10 binding molecules of the invention are also useful for inhibiting the proliferation of, or killing tumor cells. For example, in one embodiment, the present invention provides a method of inhibiting the proliferation of tumor cells, the method comprising contacting tumor cells with an ADAM10 binding molecule, or a composition comprising an ADAM10 binding molecule, such as, for example, a pharmaceutical composition. In some such embodiments, the tumor cells are colorectal tumor cells, colon tumor cells, breast tumor cells, ovarian tumor cells, lung tumor cells, brain tumor cells, glioma cells, glioblastoma cells, or neuroblastoma cells. In some embodiments the cells are in vitro. In some embodiments the cells are in vivo.

In other embodiments, the present in the present invention provides methods of inhibiting (a) ADAM10-dependent proteolytic cleavage of an ADAM10 substrate, and/or (b) binding of ADAM10 to an ADAM10 substrate, and/or (c) activation (e.g., transactivation) of and ADAM10 substrate, and/or (d) signaling by an ADAM10 substrate whose signaling activity is modulated by ADAM10-dependent proteolytic activity, such methods comprising contacting cells with an ADAM10 binding molecule, or a composition comprising an ADAM10 binding molecule. In some such embodiments the cells are tumor cells, such as, for example, colorectal tumor cells, colon tumor cells, breast tumor cells, ovarian tumor cells, lung tumor cells, brain tumor cells, glioma cells, glioblastoma cells, or neuroblastoma cells. In some embodiments the cells are in vitro. In some embodiments the cells are in vivo.

In the case of in vivo therapeutic applications, clinical response to administration of an ADAM10 binding molecule can be assessed using standard screening techniques known in the art, such as magnetic resonance imaging (MRI), x-radiographic imaging, computed tomographic (CT) scan, flow cytometry or fluorescence-activated cell sorter (FACS) analysis, histology, gross pathology, and blood chemistry, including but not limited to changes detectable by ELISA, ELISPOT, RIA, chromatography, and the like. Further, the subject undergoing therapy with the ADAM10 binding molecule can experience improvement in the symptoms associated with the disease being treated.

Methods of preparing ADAM10 binding molecules for administration to a subject, and methods of administering an ADAM10 binding molecule to a subject, are well-known to those of ordinary skill in the art, or can be readily determined by those of ordinary skill in the art. For example, the route of administration of the ADAM10 binding molecule can be, for example, oral, parenteral, by inhalation, or topical. The term “parenteral” as used herein includes, e.g., intravenous, intraarterial, intraperitoneal, intramuscular, subcutaneous, rectal, and vaginal administration. Oral dosage forms include, e.g., capsules, tablets, aqueous suspensions, and solutions. Nasal aerosol or inhalation dosage forms can be prepared, for example, as solutions in saline, employing benzyl alcohol or other suitable preservatives, absorption promoters to enhance bioavailability, and/or other conventional solubilizing or dispersing agents.

Usually, a suitable pharmaceutical composition can comprise a buffer (e.g., acetate, phosphate or citrate buffer), optionally a surfactant (e.g., polysorbate), optionally a stabilizer agent (e.g., human albumin), etc. The form and character of the pharmaceutically acceptable carrier or diluent can be dictated by the amount of active ingredient with which it is to be combined, the route of administration and other well-known variables. A cocktail comprising one or more species of ADAM10 binding molecules, e.g., anti-ADAM10 antibodies, or antigen-binding fragments or variants thereof, can also be used. In other methods, ADAM10 binding molecules can be delivered directly to the site where its action is required, thereby increasing the exposure of the target cells (e.g., tumor cells) to the therapeutic agent. In one embodiment, the administration is directly into a tumor.

As discussed herein, ADAM10 binding molecules can be administered in a therapeutically effective amount for the in vivo treatment of certain cancers, such as breast cancer, colon cancer, lung cancer, and gliomas. In this regard, it will be appreciated that the disclosed binding molecules can be formulated to facilitate administration and promote stability of the ADAM10 binding molecules. Pharmaceutical compositions in accordance with the present invention can comprise a pharmaceutically acceptable, non-toxic, sterile carrier such as physiological saline, non-toxic buffers, preservatives and the like. For the purposes of the instant application, a “therapeutically effective amount” of an ADAM10 binding molecule means an amount sufficient to achieve a benefit, e.g., to ameliorate symptoms of a disease or condition (e.g., a cancer) or to inhibit proliferation of a cancer cell. Suitable formulations for use in the therapeutic methods disclosed herein are described in Remington's Pharmaceutical Sciences (2000).

The composition can be administered as a single dose, multiple doses, or over an established period of time in an infusion. Dosage regimens also can be adjusted to provide the optimum desired response (e.g., a therapeutic or prophylactic response). The amount of an ADAM10 binding molecule that can be combined with carrier materials to produce a dosage form will vary depending upon many different factors, including means of administration, target site, physiological state of the patient (i.e., the severity of the disease, the history of the disease, and the age, height, weight, health, and physical condition of the individual undergoing therapy), whether treatment is prophylactic or therapeutic, other medications administered, and whether the subject is a human or an animal. Usually, the subject is a human, but non-human mammals, including transgenic mammals, can also be treated. The amount of an ADAM10 binding molecule to be administered is readily determined by one of ordinary skill in the art without undue experimentation, given this disclosure. Treatment dosages can be titrated using routine methods known to those of skill in the art to optimize safety and efficacy.

The ADAM10 binding molecules of the invention can be administered in combination with one or more additional active agents. For example, in the treatment or prevention of recurrence of cancer, the ADAM10 binding molecule can be administered in conjunction with a standard-of-care (SOC) agent. In some instances, the ADAM10 binding molecule is administered in combination with one or more chemotherapeutic agents or other therapeutic agents, including immunotherapeutic agents. Examples of other agents that can be co-administered with an ADAM10 binding molecule include, but are not limited to, afatinib, actinomycin, azacitidine, azathioprine, bevacizumab, bleomycin, bortezomib, carboplatin, capecitabine, cetuximab, cisplatin, chlorambucil, cyclophosphamide, cytarabine, daunorubicin, docetaxel, doxifluridine, doxorubicin, epirubicin, epothilone, erlotinib, etoposide, fluorouracil, gefitinib, gemcitabine, hydroxyurea, idarubicin, imatinib, irinotecan, mechlorethamine, mercaptopurine, methotrexate, mitoxantrone, olaparib, oxaliplatin, paclitaxel, panitumab, pazopanib, pemetrexed, poly(ADP-ribose) polymerase (PARP) inhibitors, tamoxifen, teniposide, tioguanine, topotecan, trastuzumab, tretinoin, valrubicin, vinblastine, vincristine, vindesine, vinorelbine, and vintafolide.

In embodiments in which more than one active agent is administered, the agents can be administered together (for example, in the same formulation and/or at the same time), or separately (for example, in different formulations and/or at different times). In some such embodiments, the agents are administered systemically. In some such embodiments, the agents are administered locally. In some such embodiments, one (or more) agent is administered systemically, and one (or more) agent is administered locally. Where two such agents are used, it may be possible to use lower dosages or amounts of each agent, as compared to the dosages necessary when each agent is used alone.

This disclosure also provides for the use of an ADAM10 binding molecule as described herein to treat or prevent recurrence of cancer, such as colon cancer, breast cancer, ovarian cancer, glioma, or lung cancer.

This disclosure also provides for the use of an ADAM10 binding molecule as described herein in the manufacture of a medicament for treating, or preventing recurrence of, a cancer, such as colon cancer, breast cancer, ovarian cancer, glioma, or lung cancer.

### Assays and Diagnostics

The ADAM10 binding molecules of the invention can also be used for a variety of different applications, including those that involve detecting ADAM10 Such methods typically involve assaying the expression level ADAM10, for example by qualitatively or quantitatively measuring or estimating the level of ADAM10 in a first biological sample either directly (e.g., by determining or estimating absolute protein level) or relatively (e.g., by comparison to a second biological sample). For example, the ADAM10 expression level in a first biological sample can be measured or estimated and compared to a that of a standard or control taken from a second biological sample A “biological sample” is a sample obtained from an individual, cell line, tissue culture, or other source of cells potentially expressing ADAM10. Methods for obtaining tissue biopsies and body fluids from mammals are known in the art. The ADAM10 binding molecules of the invention can be used to assay ADAM10 protein levels in a biological sample using classical immunohistological methods known to those of skill in the art (see, e.g., Jalkanen et al., (1985); Jalkanen et al., 1987). Immunoassays that can be used include but are not limited to competitive and non-competitive assay systems using techniques such as Western blots, radioimmunoassays, ELISA (enzyme linked immunosorbent assay), ELISPOT, “sandwich” immunoassays, immunoprecipitation assays, precipitin reactions, gel diffusion precipitin reactions, immunodiffusion assays, agglutination assays, complement-fixation assay s, immunoradiometric assays, fluorescent immunoassays, protein A immunoassays, and immunoelectron microscopy, to name some examples. Such assays are routine and well known in the art. Those skilled in the art will be able to determine operative and optimal assay conditions for each determination by employing routine experimentation.

Detection of ADAM10 can be facilitated by coupling the binding molecule to a detectable substance or label. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin. An example of a luminescent material is luminol. Examples of bioluminescent materials include luciferase, luciferin, and aequorin. Examples of suitable radioactive material include 125I, 131I, 35S, or 3H.

In situ detection can be accomplished by removing a histological specimen, for example a tumor sample, from a subject, and contacting the specimen with a labeled ADAM10 binding molecule, or with an ADAM10 antibody and a labeled secondary antibody. Through the use of such a procedure, it is possible to determine not only the presence of ADAM10, but also its distribution in the examined tissue. Using the present invention, those of ordinary skill will readily perceive that any of a wide variety of histological methods (such as staining procedures) can be modified in order to achieve such in situ detection.

### Kits Comprising ADAM10 Binding Molecules

This disclosure further provides kits that comprise an ADAM10 binding molecule, which can be used to perform the methods described herein. In certain embodiments, a kit comprises at least one purified ADAM10 binding molecule in one or more containers. In some embodiments, the kit contains one or more of the components necessary and/or sufficient to perform a detection assay, including controls, directions for performing assays, and any necessary software for analysis and presentation of results. One skilled in the art will readily recognize that the disclosed ADAM10 binding molecules can be readily incorporated into any of the established kit formats that are well known in the art.

Embodiments of the present disclosure can be further described and understood by reference to the following non-limiting “Examples,” which describe in the preparation of certain exemplary ADAM10 binding molecules, some exemplary characterization of such molecules, and some exemplary methods for using such binding molecules. It will be apparent to those skilled in the art that many modifications to the specific description provided in the Examples can be practiced without undue experimentation and without departing from the scope of the present disclosure.

### EXAMPLES

### Example 1: Expression and Purification of ADAM10 Constructs

ADAM10 constructs used in these Examples were expressed and purified as follows.

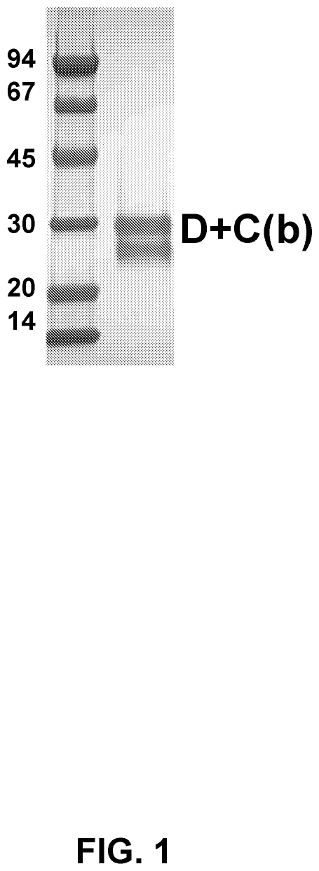

Bovine ADAM10 ECD (20-646) and the D+C domains (455-646) were cloned, expressed and purified from HEK293 cells using a custom made pcDNA™ 3.1+ vector (Janes et al, 2005). The constructs were fused to a removable Fc-tag at the C-terminus. The C-terminal Fc-tag was used to facilitate protein-A affinity chromatography and removed by thrombin cleavage afterwards. SDS-PAGE profile of the purified bovine ADAM10 D+C is shown in FIG. 1. The human ADAM19 D+C construct (438-646) was likewise purified using the same mammalian expression system.

Human ADAM10 ECD (20-650), human ADAM10 D+C (455-650), human ADAM17 ECD (20-655) and the human ADAM17 D+C (482-648) were cloned into a custom-made pMA152a baculovirus vector (Xu et al., 2008). pMA152a is based on the pAcGP67B vector (BD Biosciences) with an incorporated removable Fc-tag (human). Secreted recombinant proteins were produced by baculovirus-infected Hi5 insect cell following the protocol provided by BD Biosciences. The proteins were purified from the culture supernatants of the insect cells using protein-A. Size-exclusion chromatography was performed as the final step to obtain all the purified recombinant proteins.

### Example 2: Generation of Fully Human Anti-ADAM10 Antibody 1H5

The bovine ADAM10 D+C was used as an antigen to pan a large naïve human Fab library. Three rounds of phage panning were conducted.

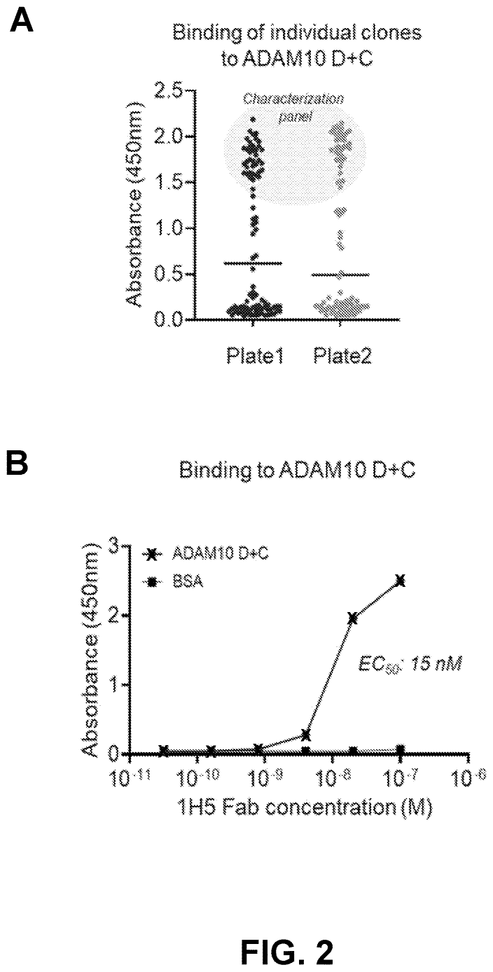

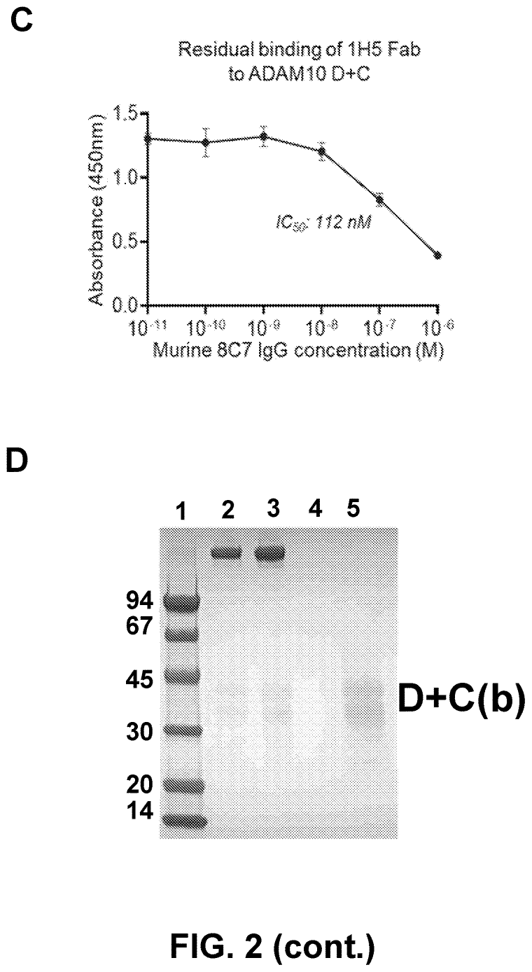

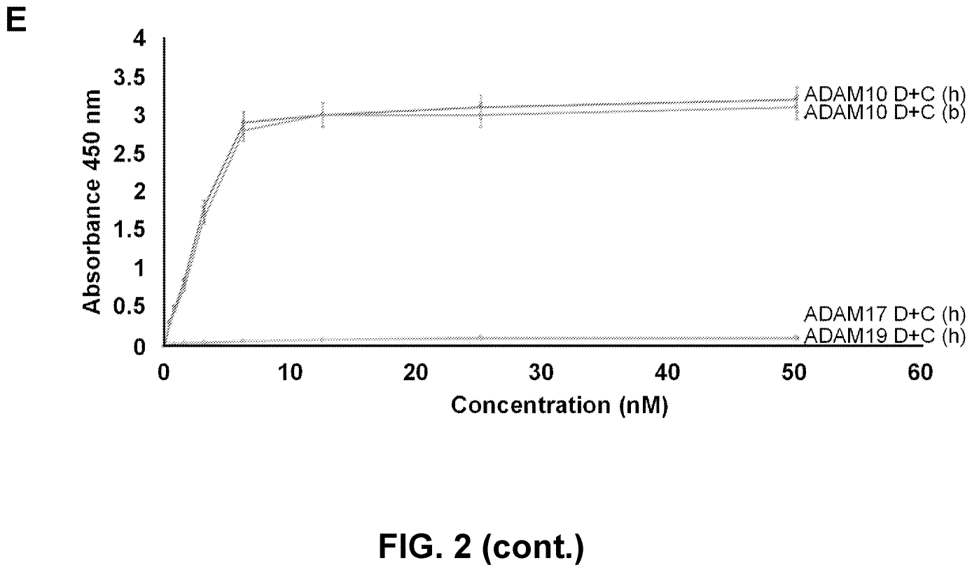

For panning, two ICAT5 and ICAT5-1 phage libraries were pre-blocked with 3% skim-milk in PBS (w/v) for 1 hour at 25° C. Blocked phages were incubated with 100 nM biotinylated human ADAM10 D+C for 1 hour at 25° C. Bound phages were separated by streptavidin coated magnetic beads and washed with PBS pH 7.4 containing 0.1% Tween-20 (w/v). Elution of bound phages was conducted by adding 1 μM of either non-biotinylated antigen for O/N at 4° C. For the second and third rounds of panning, reduced concentration (50 nM and 5 nM respectively) of biotinylated ADAM10 D+C was applied to pan out high-affinity Fab binders. After the three rounds of panning, binding of 192 individual clones were analyzed in ELISA, and the selected clones after rescue of pCAT2 plasmid DNA were sequenced. A dominant clone named 1H5 was identified (FIG. 2, Panel A). 1H5 Fab and IgG were purified as described previously (Baek et al., 2022).

Competitive ELISA was used to gauge relative bindings of 1H5 and murine mAb 8C7 to immobilized ADAM10 D+C domains construct (antigen). To perform the competitive ELISA, wells were coated with 100 μl of ADAM10 D+C (concentration 2 μg/ml). After washes with phosphate buffer solution (PBS), the wells were blocked with 4% non-fat dry milk. Varying concentrations of 1H5 or 8C7 were added to the wells and incubated for 1 hour. Goat anti-human IgG cross-adsorbed secondary antibody conjugated to horseradish peroxidase (HRP) (1:2000 dilution) was used to detect the binding of 1H5 to immobilized ADAM10 D+C antigen. Rabbit anti-mouse IgG cross-adsorbed secondary antibody conjugated to HRP (1:2000 dilution) was used to detect binding of 8C7 to ADAM10 D+C antigen. Cross-adsorption ensured that the secondary antibodies maintained their desired species reactivity as shown in the graph (1H5/anti-murine secondary or 8C7/anti-human secondary). Color was developed using a TMB substrate kit and data was recorded at 450 nm. For the competition assay, a mixture of 11H5 and 8C7 at a 1:1 molar ratio was used. The final concentration of each mAb in the mixture was the same as when added individually. The mixture was added to the wells and incubated for 1 hour. Relative binding of 1H5 or 8C7 (in the mixture) to ADAM10 D+C was detected using goat anti-human IgG or rabbit anti-mouse IgG secondary (cross-adsorbed, 1:2000 dilution) conjugated to HRP. The ELISA confirmed that 1H5 bound to ADAM10 D+C in a concentration dependent manner. In addition, competitive ELISA experiments with the murine 8C7 IgG demonstrated that 1H5 binds similar epitope region in ADAM10 (see FIG. 2, Panels A-C).

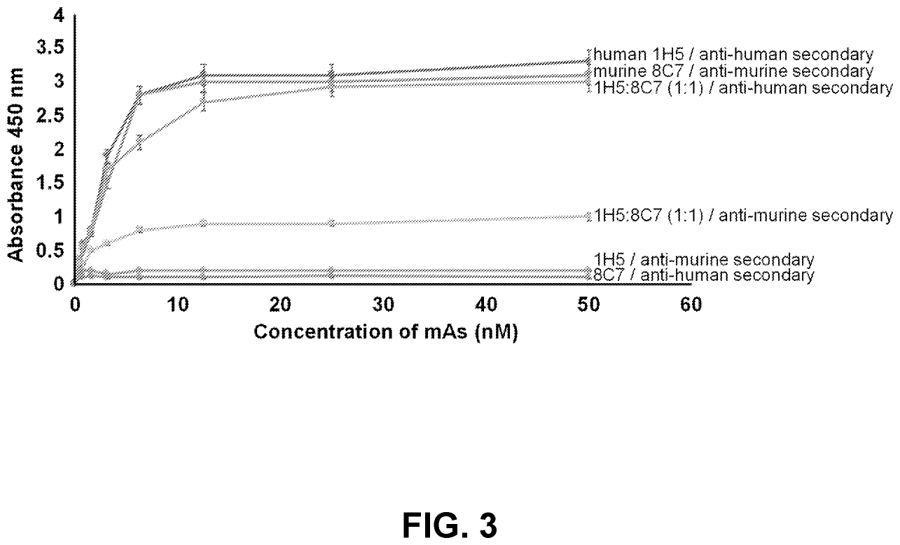

Additional ELISA experiments were performed to confirm the binding competition of the human 1H5 and the murine 8C7 to immobilized ADAM10 D+C antigen. The results shown in FIG. 3 document that both 1H5 and 8C7 efficiently bind to ADAM10 D+C when added alone, and that 1H5 outcompetes 8C7 when added at a 1:1 molar ratio mixture. Using cross-adsorbed secondary antibodies, it was ascertained that there were no cross-species reactivity as evidenced from the 1H5/anti-murine secondary or 8C7/anti-human secondary plots.

### Example 3: 1H5 Inhibits Proliferation of Colon Cancer Cell Lines

Alamar blue cell viability assays were used to evaluate the anti-proliferative potential of the fully human mAb 1H5 using a variety of cancer cell lines that include colon, breast, ovarian, glioma and lung adenocarcinoma (non-small cell lung cancer or NSCLC).

The colon cancer cell lines LIM1215 and COLO205 display high levels of Notch receptors (Pal et al., 2015) The breast cancer cell line MDA-MB-231 is triple-negative expressing EGFR while the SKBR-3 overexpresses HER (Subik et al., 2010). The ovarian cancer cell line OVCAR-3 represent high-grade serous carcinoma (HGSC) type, while SKOV-3 belongs to the non-HGSC type (Potts et al., 2019). Though high-grade serous carcinoma is the most prevalent amongst ovarian cancer patients, the non-serous types are known to migrate and invade more aggressively (Potts et al., 2019). Notch1 is known to be activated in glioblastoma and it has been shown that targeting Notch1 suppressed growth and proliferation of U-87 MG in vitro and in xenograft models (Long e al., 2018). EGFR signaling plays a crucial role in non-small cell lung cancer (NSCLC) occurrence and progression and is increased in over 45% of the tumor lesions from NSCLC patients (Rusch et al., 1993). The objective was to evaluate if the 1H5 can inhibit the proliferation of these cancer cell lines that are either Notch or EGFR/HER2 dependent.

For the Alamar blue cell viability assays, the cells were harvested in the log phase of growth (after 3 days of culturing). The cell count was determined and was adjusted to 5×10⁴ cells/ml. The cells were allowed to adhere and grow for 24 hours in 96-well cell culture plates, treated with test agent, in this case, 1H5. The cells were allowed to grow for an additional 38 hours. Cells not treated with 1H5 were used as a control. Alamar Blue (10% of the well volume) was added aseptically. Cultures containing Alamar Blue were incubated for 6 hours, and cell proliferation was measured spectrophotometrically by absorbance at 570 and 600 nm. Cell viability was calculated using the following formula:

$$Percentage difference between treated and control cells = \frac{( O 2 \times A 1 ) - ( O 1 \times A 2 )}{( O 2 \times P 1 ) - ( O 1 \times P 2 )} \times 100$$

O1=molar extinction coefficient (E) of oxidized alamarBlue® (Blue) at 570 nm, O2=E of oxidized alamarBlue® at 600 nm, A1=absorbance of test wells at 570 nm, A2=absorbance of test wells at 600 nm, P1=absorbance of positive growth control well (cells plus AamarBlue® but no test agent) at 570 nm, P2=absorbance of positive growth control well (cells plus AlamarBlue® but no test agent) at 600 nm (O'Brien et al., 2000).

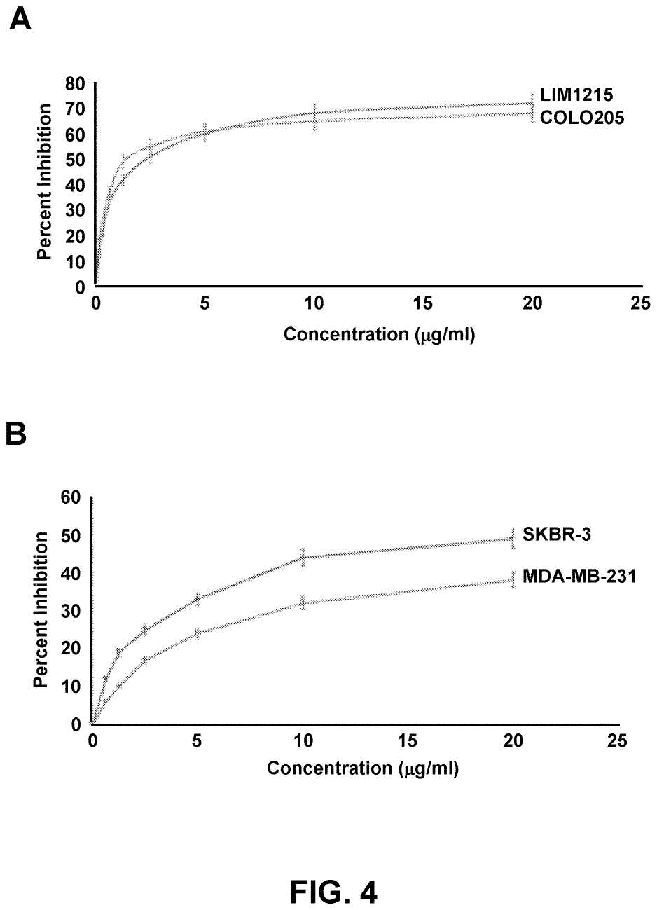

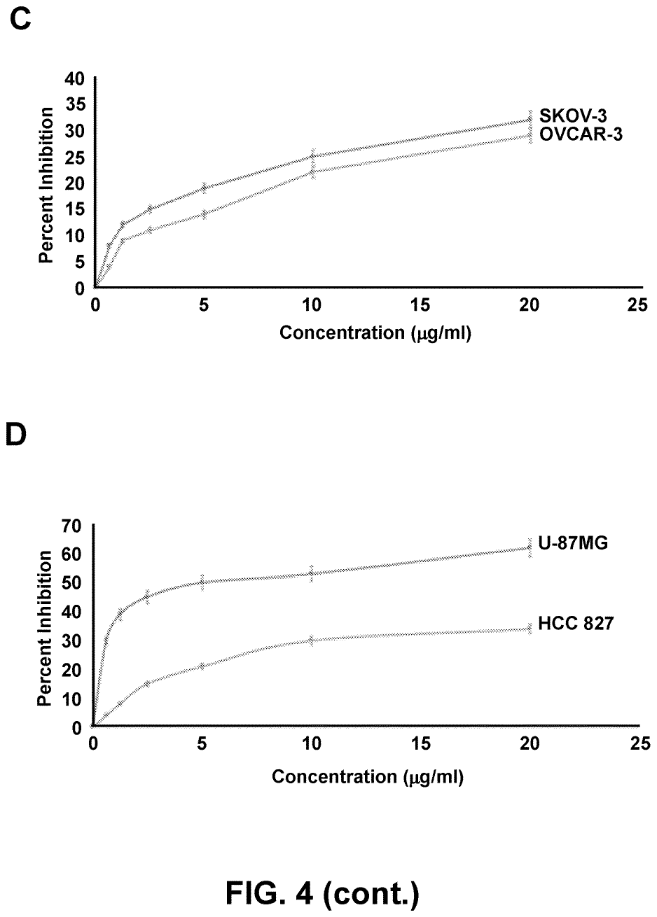

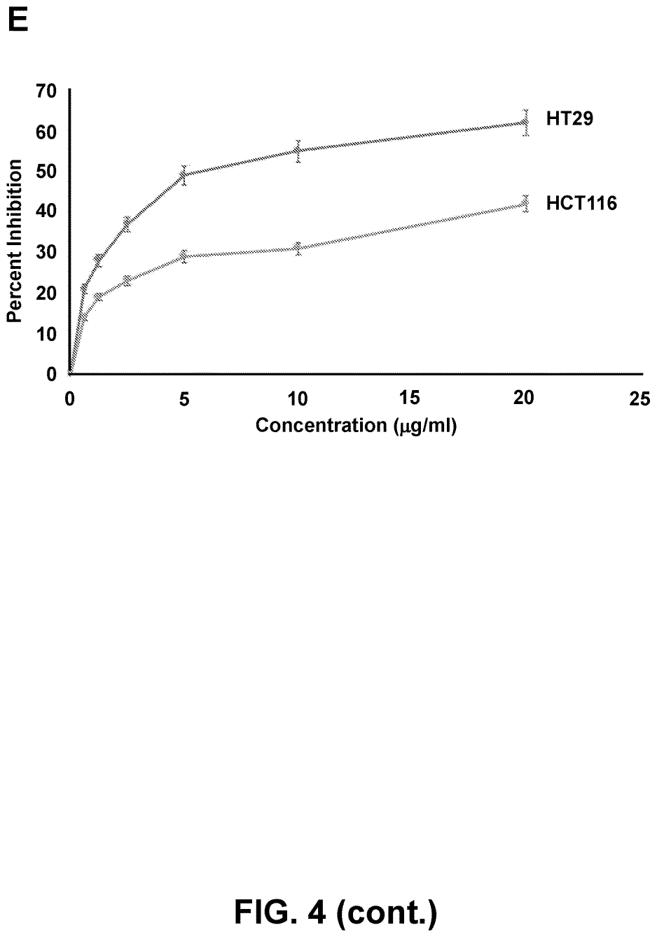

The Alamar blue assays show that 1H5 is more efficient in inhibiting Notch-dependent cancer cell lines, including LIM1215, COLO205 and U-87MG (60-70% inhibition at 20 μg/ml), as compared to EGFR/HER2 dependent lines, such as MDA-MB-231, SKBR-3, OVCAR-3, SKOV-3 or HCC-827 (2545% inhibition at 20 μg/ml) (see FIG. 4).

Using the Alamar blue assay, the anti-proliferative effects of 1H5 was further studied in two other colon cancer lines: HT-29, which contains a BR4F mutation (V600E), and HTC116, which harbors a mutation in the codon 13 of Kirsten rat sarcoma virus (KR4S) gene. The results of the assay shows 1H5 inhibited growth of both of the colon cancer cell lines (FIG. 4, Panel E).

### Example 4: 1H5 Causes Tumor Growth Inhibition of Colon Cancer Cell Line

The in vivo anti-tumor efficacy of mAb 1H5 in a mouse model of CRC using xenografted COLO205 cells was studied.

In a first study, both 1H5 alone and in combination with irinotecan, a chemotherapeutic drug used for the treatment of CRC (Atapattu et al., 2016), was used in the mouse model.

For the mouse model, COLO205 cells were grown in monolayer culture, harvested by trypsinization, and implanted subcutaneously into the right flank of 6- to 8-week-old female athymic nude mice. Approximately 10 million cells were injected per mouse. Mice were randomized into 4 groups (each n=4). When tumor volumes reached ˜100-150 mm³, the four groups were injected as follows:
Group 1: 1H5 (i.p.), 30 mg/kg, biweekly (total 7 doses)
Group 2: Irinotecan (i.p.), 20 mg/kg, three doses, once a week starting day 12
Group 3: Irinotecan (20 mg/kg, i.p., once a week starting day 12, total three doses)+continued 1H5 treatment (30 mg/kg, i.p., biweekly, total 7 doses).
Group 4: sterile PBS (as a control).

Tumor volume was determined by external caliper and calculated by the modified ellipsoidal formula: V=½(Length×Width²). Antitumor efficacy was calculated as (1-dT/dC)λ100, where dT is the final tumor volume minus the starting tumor volume from the treatment group, and dC is the final tumor volume minus the starting tumor volume of the control group (Rios-Doria et al., 2015). Error bars were calculated as SEM. The mouse body weight and general health were monitored daily.

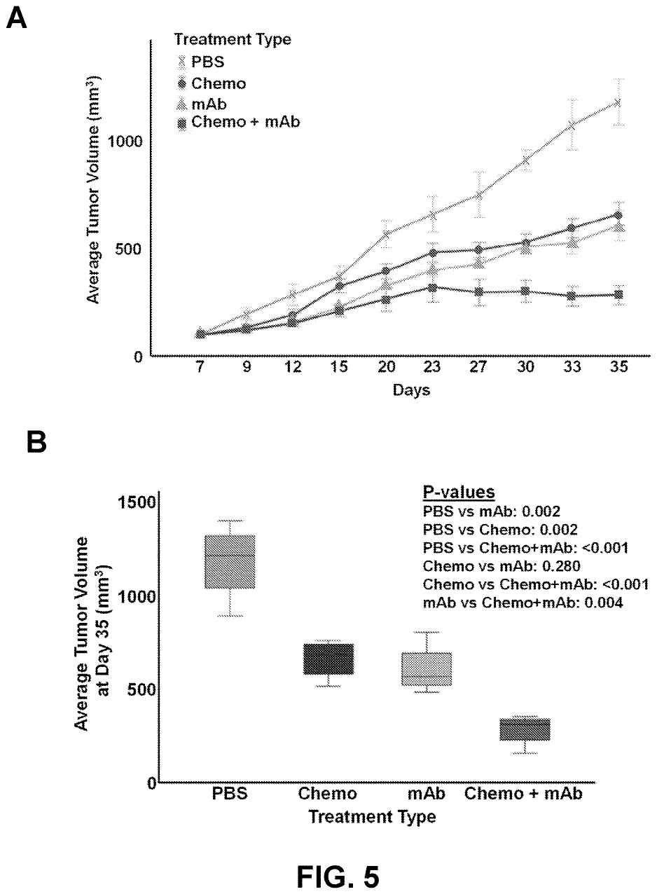

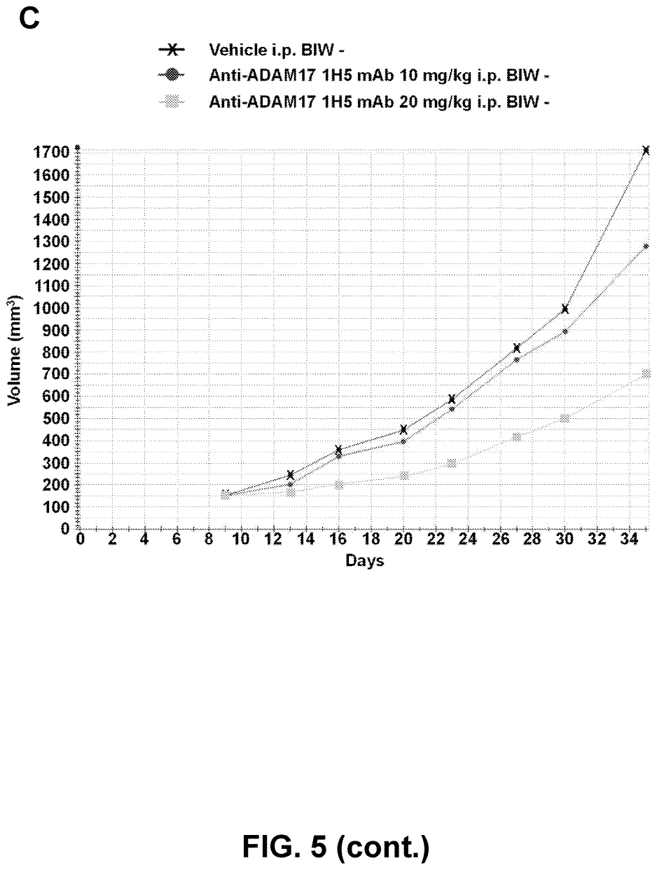

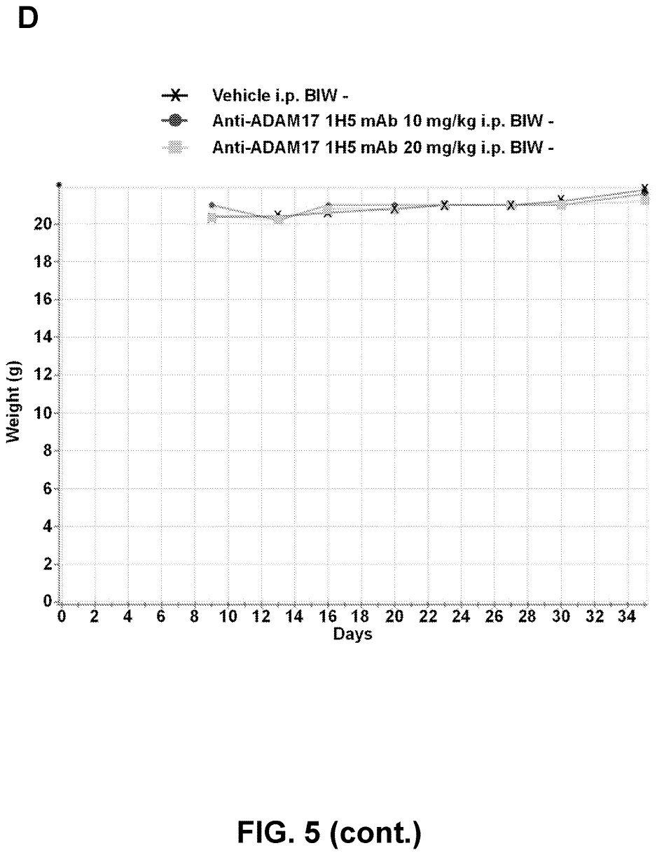

In the combination treatment, there was 83% tumor growth inhibition as compared to 54% for 1H5 alone and 48% for Irinotecan alone (FIG. 5. Panel A). Independent t test analysis showed significant difference in tumor volume reduction (day 35) between the control (sterile PBS) and all treatments (for 1H5 and irinotecan alone, p=0.002; for combined 1H5 and irinotecan treatment, p<0.001) (FIG. 5, Panel B). Significant tumor volume reduction differences were also observed between irinotecan alone and the combined treatment (p<0.001), and between 1H5 treatment alone and combined treatment (p=0.004). There were no toxicity effects in animals, gauged from no loss in mouse weight or presence of visible diarrhea, despite 1H5 binding equally well to human and mouse ADAM10 in ELISA-based assays (data not shown). This con firms that despite ADAM10 being present on a variety of cells, 1H5 selectively targets the tumors without any significant side effects or toxicity.

In a second study, 1H5 at two different doses was used in the mouse model. For the mouse model, approximately five million cells were implanted (subcutaneous with Matrigel) into 6- to 8-week-old female athymic nude mice. Mice were randomized into 3 groups (each n=5). When tumor volumes reached ˜150 mm³, the three groups were injected as follows:
Group 1: 1H5 (i.p.), 10 mg/kg, biweekly (total 8 doses)
Group 2: 1H5 (i.p.), 20 mg/kg, biweekly (total 8 doses)
Group 3: sterile PBS (as a control).

Both 1H5 doses inhibited tumor growth, with the 20 mg/kg dose demonstrating a greater inhibitory effect (64%) than the 10 mg/kg dose (26%) (FIG. 5. Panel C). In addition, both 11H5 doses did not affect the weight of the mice during the course of the experiment (FIG. 5, Panel D).

### Example 5: 1H5 Binds to ADAM10 Domain

To study the crystal structure of the ADAM10 ECD, a bovine ADAM10 fragment containing the disintregrin and cysteine-rich domains (ADAM10 D+C, residues 455-646) was produced as described previously (Janes et al., 2005). The 1H5 Fab fragment was prepared by digesting 1H5 with papain at pH 6.5 (enzyme/substrate ratio 1:100) for 2 hours at room temperature. The final purification was performed using gel filtration chromatography (SD-200 column, 20 mM Hepes, and 150 mM NaCl, pH 7.5). The protein eluted as a monomer of ˜50 kDa. For crystallization, ADAM10 D+C was mixed with the 1H5 Fab at 1:1 molar ratio (final concentration 22 mg/ml) in a buffer containing 20 mM HEPES, 150 mM NaCl, pH 7.4. The complex was crystallized in a hanging drop by vapor diffusion at room temperature against a reservoir containing 0.1M BICINE, pH 8.5, 20% PEG10,000. The initial thin plate like crystals were optimized using streak seeding. Sizeable crystals, in the space group P21221, were obtained and data was collected. The structure was determined using molecular replacement with the ADAM10 D+C/8C7-F(ab′)2 structure as a search model (PDBID 5L0Q). The ADAM10/1H5 structure model was built with the program Coot and refined with PHENIX_Refine. The final structure was validated with PROCHECK.

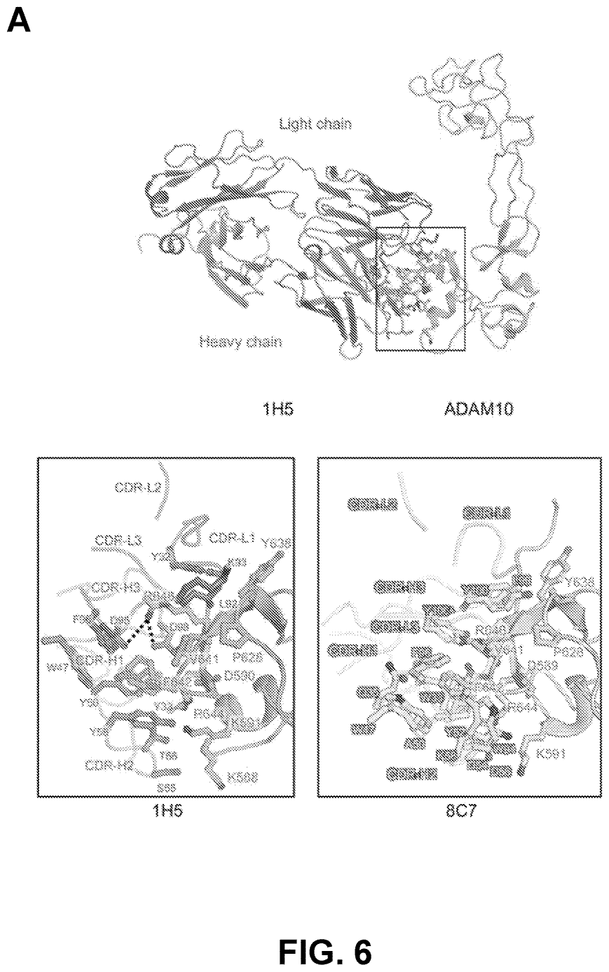

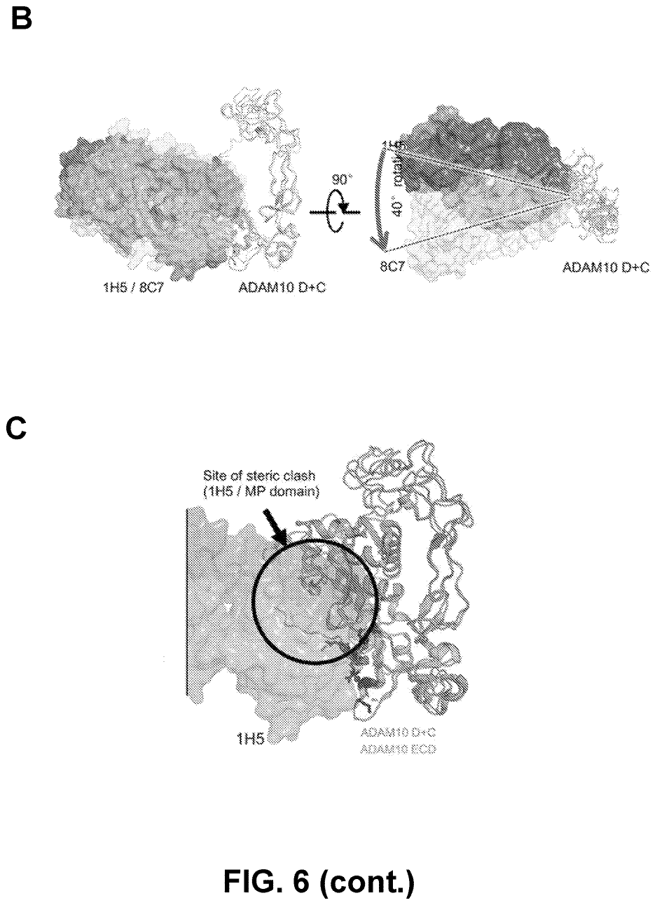

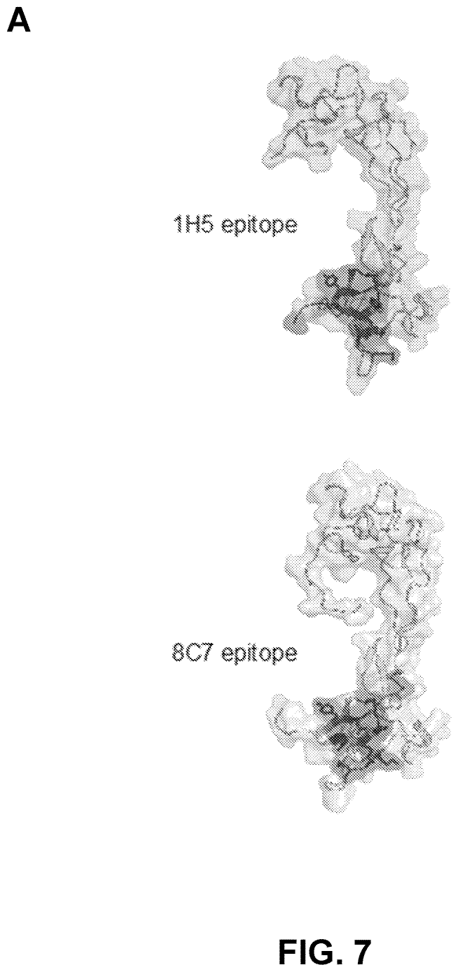

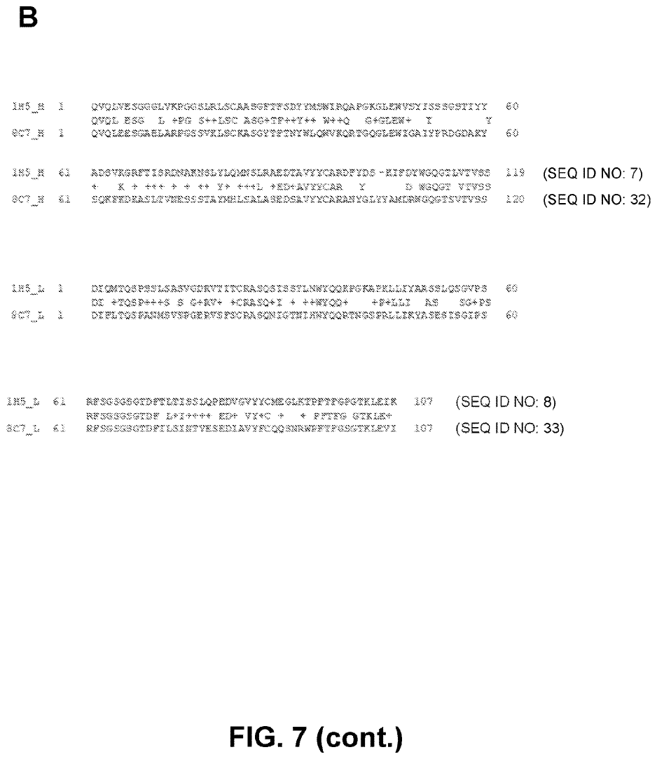

The crystal structure of the ADAM10 ECD revealed its ‘closed’, auto-inhibited conformation, where the MP and C domains are in contact, partially obscuring the catalytic cleft, and also the substrate-interacting C domain region (Seegar et al., 2017). Importantly, the previously determined crystal structure of the murine 8C7 bound to the isolated ADAM10 D+C domain region showed that 8C7 binds a defined epitope in the substrate-interacting C domain (Atapattu et al., 2012), which would be inaccessible in the auto-inhibited ADAM10 conformation. To address how 1H5 binds ADAM10, and to compare it to 8C7, the structure of ADAM10 D+C was determined in complex with the isolated Fab fragment of 1H5 at 3.8 Å resolution (see FIGS. 6 and 7; see also Table 4). The structure reveals that 1H5 binds ADAM10 at an epitope similar to 8C7 (FIG. 7), but with a distinct recognition strategy and approaching angle (FIG. 6, Panels A and B).

Formation of the 11H5 Fab/ADAM10 D+C complex buries ˜791 Å² of surface area in each molecule. The antibody CDRs target the C domain of ADAM10 as expected, via residues on the first and third CDR of the light chain (CDR-1 and CDR-3) and heavy-chain CDR-1 to CDR-3. The center of this interface is formed by embedding of three hydrophobic ADAM10 residues, V₆₄₁ and F₆₄₂ and P₆₂₈, into a hydrophobic groove defined by 1H5 light chain CDR-1 and CDR-3 residues (Y₃₂, L₉₂, K₉₃, and F₉₆) and by heavy chain CDR-1 and CDR-3 and framework residues (W₃₃, W₄₇, Y₅₀, and Y₅₈). Adjacent to this hydrophobic core, heavy chain CDR-3 residues D₉₅ and D₉₈ form salt-bridges with ADAM10 residue R₆₄₆. There are multiple hydrogen bonds in the surrounding regions that further stabilize the interaction, including hydrogen bonds between R₆₄₄ (ADAM10) and Y₃₃, Y₅₀, and T₅₆ (heavy chain CDR-1 and CDR-2); between K₅₉ (ADAM10) and Y₅₈ (heavy chain CDR-2); and between D₅₉₀ (ADAM10) and Y₃₃ (heavy chain CDR-1).

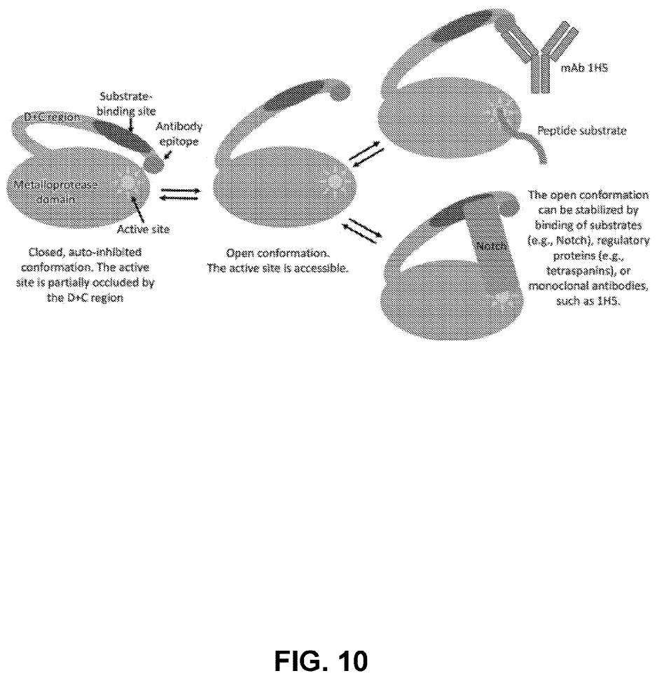

The data also reveals that the ADAM10 D+C structure in the 1H5/ADAM10 complex is very similar to that in the 8C7/ADAM10 complex structure (Atapattu et al., 2016), as well as in the structures of the unbound ADAM10 D+C (Janes et al., 2005) and ADAM10 ECD (Seegar et al, 2017). The 1H5-bound and 8C7-bound ADAM10 D+C structures can be superimposed with a root-mean-square deviation (r.m.s.d.) of 1.675 Å between 185 Cα atoms (FIG. 6, Panel B). Importantly, binding of 1H5, like 8C7, would not be compatible with the autoinhibited ADAM10 conformation, as it would sterically clash with the D+C region-interacting M domain in the autoinhibited state (Seegar et al., 2017) (FIG. 6, Panel C; vee also FIG. 10). Consistent with these observations, 1H5 binds the isolated recombinant ADAM10 D+C region with a K_{D} of 3.3 nM, and the full recombinant ADAM10 ECD, which predominantly adopts the autoinhibited conformation in solution (Seegar et al., 2017), with a K_{D} of ˜200 nM (as measured by biolayer interferometry, data not shown). For comparison, the murine 8C7, binds the ADAM10 D+C region with a K_{D} of 14 nM and the ADAM10 ECD, with a K_{D} of ˜100 nM (Atapattu et al., 2012).

TABLE 4
X-ray crystallography data collection and refinement statistics
(values in parentheses are for highest-resolution shell).

| | 1H5:ADAM10 D + C | |
|---|---|---|
| Data collection | | |
| Wavelength | 0.9792 | |
| Resolution range (Å) | 50-2.65 | (2.70-2.65) |
| Space group | P 2₁ 2 2₁ | |
| Cell dimensions | | |
| a, b, c (Å) | 78.58 85.69 129.93 | |
| a, b, g (°) | 90, 90, 90 | |
| Unique reflections | 7886 | (627) |
| Multiplicity | 21.2 | (16.9) |
| Completeness (%) | 99.8 | (99.9) |
| I/σI | 29.9 | (2.1) |
| Wilson B-factor | 91.75 | |
| R_{merge} | 0.200 | (1.306) |
| CC_{1/2} | 0.998 | (0.896) |
| Refinement | | |
| Reflections used in refinement | 7875 | (625) |
| Reflections used for R-free | 382 | (31) |
| R_{work} | 0.29 | (0.38) |
| R_{free} | 0.31 | (0.38) |
| Number of non-hydrogen atoms | | |
| macromolecules | 4780 | |
| ligands | 4752 | |
| solvent | 28 | |
| Protein residues | 634 | |
| RMS (bonds) (Å) | 0.003 | |
| RMS (angles) (°) | 0.67 | |
| Ramachandran favored (%) | 93.13 | |
| Ramachandran outliers (%) | 0.48 | |
| Average B-factor (Å²) | | |

| | |
|---|---|
| macromolecules | 111.83 |
| ligands | 111.55 |
| solvent | 158.66 |

### Example 6: 1H5 Recognizes an Activated ADAM10 Conformation Present on Cancer Cells

Cell-based ELISA assays (Smith et al. 1997) were performed to gauge the binding of 1H5, relative to the binding of the commercial anti-ADAM10 mAb1427 (monoclonal mouse IgG2B Clone #163003) to ADAM10 expressed on the cell surface of colon cancer cell lines LIM1215 and COLO205 as well as to HEK293 cells and HEK293 cells transfected with full-length human ADAM10. The mAb1427 was shown previously to not be conformation specific (Atapattu et al., 2012; Atapattu et al., 2016).

Briefly, 5×10⁴ cells/well were immobilized on 96-well ELISA plates (Greiner bio-one) with 1% paraformaldehyde for 2 hours at 37° C. The plate was washed thrice with PBS and blocked for 2 hours at room temperature with 4% non-fat dry milk. The anti-ADAM10 mAbs were added in varying concentrations. Mouse mAb conjugated to HRP and recognizing human IgG was used as a secondary antibody (Abcam) to detect 1H5 binding to ADAM10 expressed on the cells and color was developed using the TMB substrate kit. For the commercial mAb1427 recognizing human ADAM10, goat anti-mouse IgG (H+L) secondary antibody conjugated to HRP was used. The data was recorded at 450 nm.

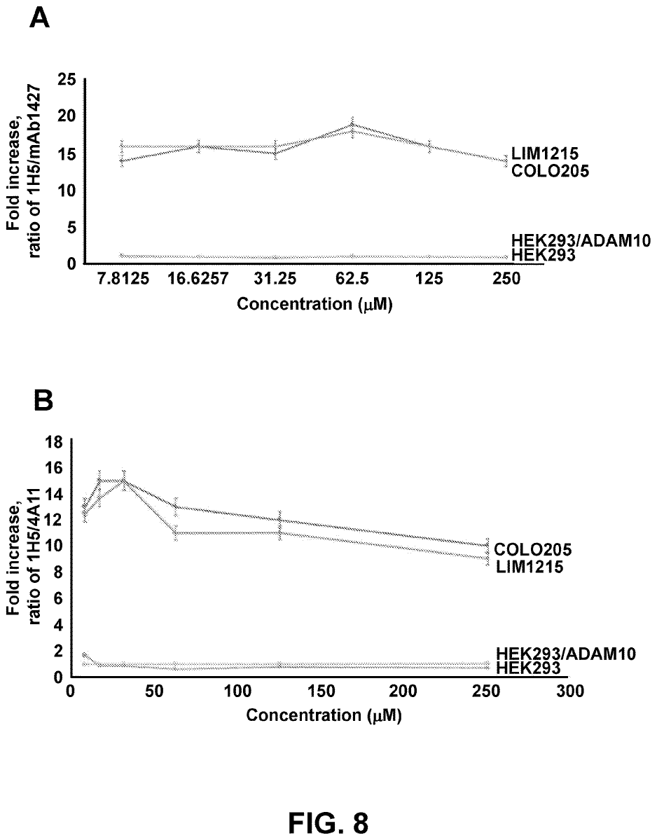

The cell-based ELISA results shown in FIG. 8 document that 1H5 preferentially binds to ADAM10 expressed on the human colon cancer cell lines, as compared to ADAM0 endogenously expressed or overexpressed on HEK293 cells. It is estimated that 1H5 binds to tumor-expressed ADAM10 approximately fourteen-fold better than to ADAM10 expressed on HEK293 cells.

### Example 7: 1H5 Stabilizes the Activated ADAM10 Conformation

The effect of 1H5 on ADAM10 catalytic activity was studied using a fluorogenic peptide cleavage assay, which used purified active human and bovine ADAM10 ECDs.

For the assay, purified 1H5, as well as 8C7 and mAb1427, were buffer-exchanged into 25 mM Tris, pH 9.0, containing 2 μM ZnCl₂, and 0.005% (w/v) Brij-35. ADAM10 ECD-antibody or ADAM10 ECD-inhibitor (broad spectrum metalloprotease inhibitor GM6001) complexes were formed at a 1:1 molar ratio prior to the assay. The assay was carried out by mixing 50 μM of a fluorogenic peptide substrate Mca-PLAQAV-Dpa with ADAM10-antibody/inhibitor complexes at 37° C., and monitoring the progress of the enzymatic reaction by fluorescence emission (excitation 320 nm and emission 405 nm) over a time course of 1 hour using a SpectraMax M5. ADAM10 ECD (bovine or human) alone was used as positive control. The substrate peptide was derived from TNFalpha and contains a highly fluorescent 7-methoxycoumarin group and a quencher group, 2,4-dinitrophenyl. ADAM10 cleaves the amide bond between the fluorescent and the quencher group causing an increase in fluorescence (Seegar et al., 2017; Black & Becherer, 1998).

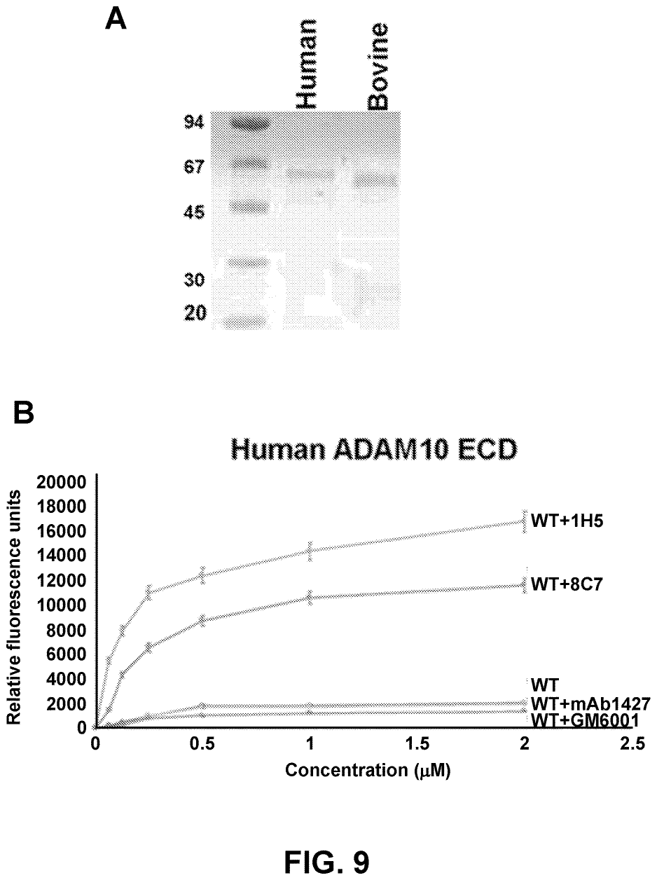

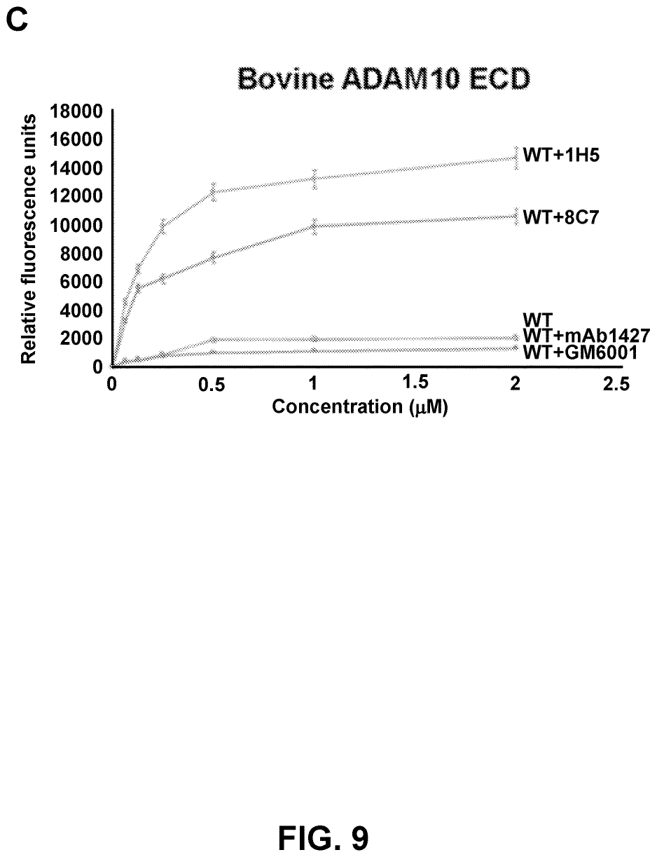

The results established that: (i) 1H5 binding stabilized activated ADAM10 and promoted the ADAM10 enzymatic activity (human or bovine): (ii) 1H5 was more efficient in augmenting the cleavage of substrate peptides than 8C7 (approximately two-folds); (iii) the commercial mAb1427 had no effect on substrate cleavage; (iv) the broad-spectrum inhibitor that is known to bind to the protease domain provided marginal inhibition (see FIG. 9). Overall, these results validated the structural observations with ADAM10 D+C bound to either 1H5 or 8C7, confirming that 1H5 binding relieves ADAM10 autoinhibition thus enhancing the catalytic activity of the enzyme.

These results support a proposed mechanism for ADAM10 activation and interactions with substrates and the 1H5 antibody (see FIG. 10). In the autoinhibited conformation, which is the predominant conformation of the ADAM10 ectodomain observed in solution (Seegar et al., 2017), the cysteine rich domain partially occludes the ADAM10 active site, hindering substrate binding. In the open ADAM10 conformation, the active site is fully accessible for substrate binding (Lipper et al., 2022). In addition to interacting with the active site, ADAM10 cell-surface substrates also interact with the D+C region of the molecule, which is required for substrate selection and/or proper substrate positioning for productive cleavage. The open ADAM10 conformation can be stabilized by binding of substrates, such as Notch, or other regulatory molecules, such as tetraspanins. The latter not only regulate the activity of ADAM10 by stabilizing the open conformation, but also selectively enhance the cleavage of certain substrate, while downregulating the cleavage of other substrates (Hartmann, et al., 2002; Murphy 2008; Janes et al., 2005, Seegar et al., 2017; Lipper et al., 2022). The 1H5 mAb, in this regard, acts in a similar fashion to the tetraspannins: it (i) selectively binds and stabilizes the activated, open ADAM10 conformation; and (ii) it selectively downregulates the cleavage of certain ADAM10 substates (Notch), while upregulating (or not affecting) the cleavage of other substrates (e.g., peptides, amyloid precursor protein (APP)).

### Example 8: 1H5 Inhibits Notch Cleavage in COLO205 Cells

Ligand-activated Notch signaling requires sequential cleavage of Notch by ADAM10 (which is the α-secretase for Notch) and by γ-secretase, releasing the Notch intracellular domain (NICD) in the cytoplasm (Hartmann et al., 2002). To confirm that 1H5 blocks Notch processing, the levels of cleaved and total (cleaved plus uncleaved) Notch1 in COLO205 cells were evaluated using sandwich ELISA.

The COLO205 cells were harvested in the log phase of growth (after 3 days of culturing), adjusted to 5×10⁴ cells/ml, and allowed to adhere and grow for 24 hours in six-well cell culture plates (Greiner bio-one). The cells were treated with 5 μg/ml of 1H5 mAb and harvested after 10, 18, and 36 hours of treatment. The PathScan® total Notch1 sandwich ELISA kit was used to measure total Notch1 according to the manufacturer's protocol (Cell Signaling Technologies). Briefly, the cells harvested at different time points were resuspended in 1X lysis buffer containing 1 mM PMSF, sonicated on ice and centrifuged for 10 min (×14,000 rpm) at 4° C. The supernatant, which is the cell lysate, was used for further study, 100 μl of the lysates (diluted to 1 mg/ml) from the different time intervals was added to the microwells (in quadruplicate) coated with a Notch1 rat antibody. This antibody captures the total Notch1 (cleaved plus uncleaved) from the lysates. After extensive washing, a Notch1 rabbit antibody was added to detect the total captured Notch1 protein. To detect Notch intracellular domain (NICD1), PathScan® cleaved Notch1 (Val1744) sandwich ELISA Kit (Cell Signaling Technologies) was used. Here, a cleaved-Notch1 rabbit detection antibody (Val1744) was used instead, to detect endogenous levels of Notch1 that is cleaved at Val1744. Anti-rabbit IgG, HRP-linked antibody was used in both cases to recognize the bound detection antibody. Color was developed using the TMB substrate. The data was recorded at 450 nm. Lysates from untreated cells served as controls. Comparison of Notch levels between treated and untreated groups was performed using independent t test.

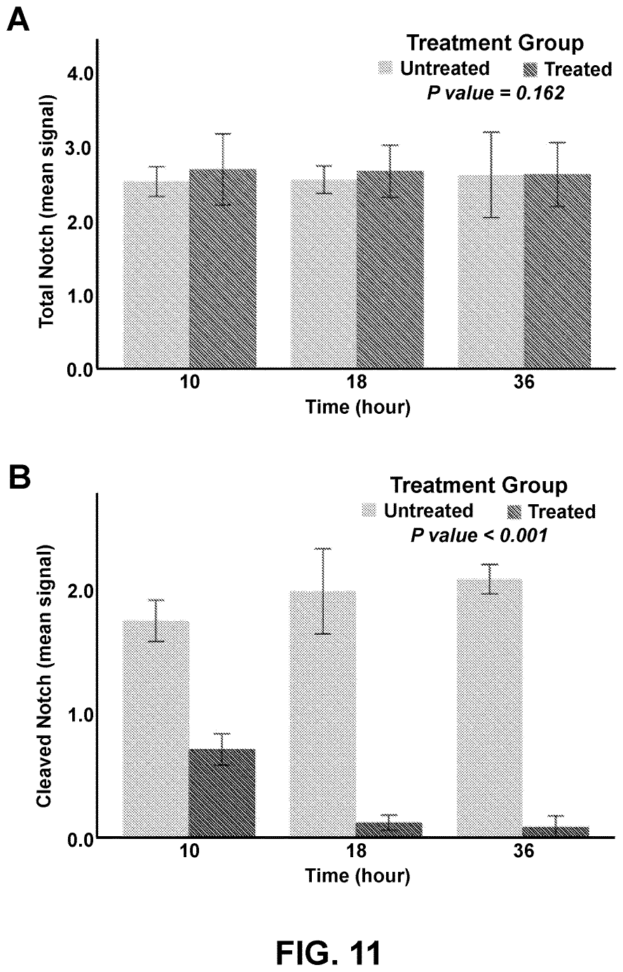

The results shown in FIG. 11 document that total notch levels did not significantly differ between the treatment and control groups (p=0.162). On the other hand, the 1H5-treated group showed significant decrease of the cleaved notch levels when compared with the untreated control (p<0.001), indicating that treatment with 1H5 significantly inhibits Notch1 cleavage without affecting the total Notch1 levels. This demonstrates the unique capacity of 1H5 to selectively inhibit notch cleavage.

### Example 9: 1H5 Augments the Cleavage of APP

The effect of 1H5 was evaluated on APP shedding using bone osteosarcoma cell line U2OS, which is a cell line that lack β-secretase but expresses endogenous ADAM10 and ADAM17 (a close relative to ADAM 10). U2OS cells transiently transfected with human APP releases the extracellular portion of APP to the media.

To investigate whether 1H5 or its Fab fragment can enhance the cleavage of the human APP ectodomain and the release of secreted amyloid precursor protein-alpha (sAPPα) in U2OS cells, the U2OS cells were transfected with N-terminally Flag-tagged full-length human APP. The release of sAPPα in the presence of varying concentrations of IHigG/Fab (31 nM to 1 μM) was evaluated using Western blotting. The Western blots were quantitated using the software Image J and glyceraldehyde 3-phosphate dehydrogenase (GAPDH) as loading control. A vector control was included and U20S cells transfected with APP, but untreated, was used to calculate the fold increase in shedding.

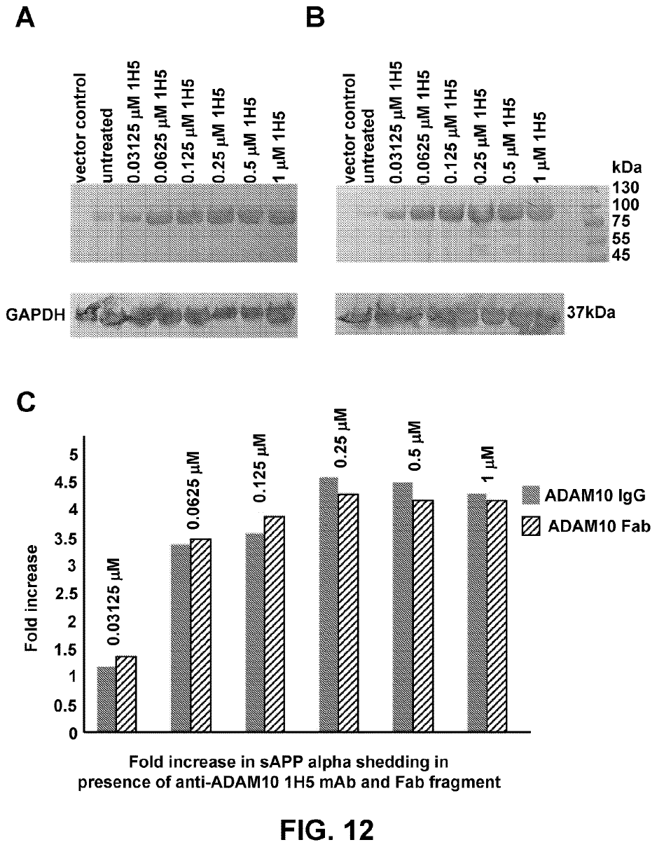

The results indicate that there is an increase in sAPPα shedding in presence of both 1H5 and its Fab fragment (FIG. 12).

### REFERENCES

Al-Lazikani B, et al. Standard conformations for the canonical structures of immunoglobulins. Journal of Molecular Biology, 1997, 273: 927-948.
Altschul S F, et al. Gapped BLAST and PSI-BLAST: a new generation of protein database search programs. Nucleic Acids Research, 1997, 25: 3389-3402.
Altschul S F & Gish W. Local alignment statistics. Methods in Enzymology, 1996, 266: 460-480.
Andersson E R & Lendahl U. Therapeutic modulation of Notch signalling—are we there yet?Nature Reviews: Drug Discovery, 2014, 13: 357-378.
Armour K L, et al. Recombinant human IgG molecules lacking Fcγ receptor I binding and monocyte triggering activities. European Journal of Immunology, 1999, 29: 2613-2624.
Atapattu L. et al. Antibodies binding the ADAM10 substrate recognition pocket inhibit Eph function. Journal of Cell Science, 2012, 125: 6084-6093.
Atapattu L, et al. An activated form of ADAM10 is tumor selective and regulates cancer stem-like cells and tumor growth. Journal of Experimental Medicine, 2016, 213(9): 1741-1757.
Back S A, et al. A new Alamar Blue viability assay to rapidly quantify oligodendrocyte death. Journal of Neuroscience Methods, 1999, 91: 47-54.
Baek D S, et al. A highly-specific fully-human antibody and CAR-T cells targeting CD66e/CEACAM5 are cytotoxic for CD66e-expressing cancer cells in vitro and in vivo. Cancer Letters, 2002, 525: 97-107.
Berzofsky J A, et al. Antibody-Antigen Interactions. In Fundamental Immunology, Paul W E, ed., Raven Press: New York, 1984.
Black R A & Becherer J D. Tumor Necrosis Factor alpha-Converting Enzyme. In Handbook of Proteolytic Emymes, Barrett A J, et al., eds., San Diego: Academic Press, 1998L 1315.
Boemer P, et al. Production of antigen-specific human monoclonal antibodies from in vitro-primed human splenocytes. Journal of Immunology, 1991, 147: 86-95.
Brennan M, et al., Preparation of bispecific antibodies by chemical recombination of monoclonal immunoglobulin G₁ fragments. Science, 1985, 229: 81-83.
Brummell D A, et al. Probing the combining site of an anti-carbohydrate antibody by saturation-mutagenesis: Role of the heavy-chain CDR3 residues. Biochemistry, 1993, 32: 1180-1187.
Burks E A, et al. In vitro scanning saturation mutagenesis of an antibody binding pocket. Proceedings of the National Academy of Sciences of the U.S.A, 1997, 94: 412-417.
Carter P, et al. Humanization of an anti-p185HER2 antibody for human cancer therapy. Proceedings of the National Academy of Science, 1992, 89: 4285-4289.
Chothia C & Lesk A M. Canonical structures for the hypervariable regions of immunoglobulins. Journal of Molecular Biology, 1987, 196: 901-917.
Clackson T, et al. Making antibody fragments using phage display libraries. Nature, 1991, 352: 624-628.
Cole et al. In Monoclonal Antibodies and Cancer Therapy, Alan R. Liss, p. 77, 1985.
Dall' Acqua W F, et al. Increasing the affinity of a human IgG1 for the neonatal Fc receptor: biological consequences. Journal of Immunology, 2002, 169: 5171-5180.
Dall' Acqua W F, et al. Properties of human IgG1s engineered for enhanced binding to the neonatal Fc receptor (FcRn). Journal of Biological Chemistry, 2006, 281: P23514-P23524.
Dikic I and Schmidt M H. Notch: implications of endogenous inhibitors for therapy. Bioessays, 2010, 32: 481-487.
Domingo-Domenech J, et al. Suppression of acquired docetaxel resistance in prostate cancer through depletion of Notch- and hedgehog-dependent tumor-initiating cells. Cancer Cell, 2012, 22: 373-388.
Fischer M, et al. Anti-DLL4 inhibits growth and reduces tumor-initiating cell frequency in colorectal tumors with oncogenic KRAS mutations. Cancer Research, 2011, 71: 1520-1525.
Gavert N, et al. Expression of L1-CAM and ADAM10 in human colon cancer cells induces metastasis. Cancer Research, 2007, 67: 7703-7712.
Gluzman Y. SV40-transformed simian cells support the replication of early SV40 mutants. Cell, 1981, 23: 175-182.
Goding. Monoclonal Antibodies: Principles and Practice, Academic Press, 1986.
Groth C & Fortini M E. Therapeutic approaches to modulating Notch signaling: current challenges and future prospects. Seminars in Cell & Developmental Biology, 2012, 23(4): 465-472.
Hartmann D B, et al. The disintegrin/metalloprotease ADAM 10 is essential for Notch signalling but not for alpha-secretase activity in fibroblasts. Human Molecular Genetics, 2002, 11: 2615-2624.
Hartmann M, et al. Who decides when to cleave an ectodomain?Trends in Biochemical Sciences. 2013, 38: 111-120.
Huse W D, et al. Generation of a large combinatorial library of the immunoglobulin repertoire in phage lambda. Science, 1989, 246: 1275-1281.
Jalkanen M, et al. Heparan sulfate proteoglycans from mouse mammary epithelial cells; localization on the cell surface with a monoclonal antibody. Journal of Cell Biology, 1985, 101: 976-985.
Jalkanen M, et al. Cell surface proteoglycan of mouse mammary epithelial cells is shed by cleavage of its matrix-binding ectodomain from its membrane-associated domain. Journal of Cell Biology, 1987, 105: 3087-3096.
Janes P W, et al. Adam meets Eph: an ADAM substrate recognition module acts as a molecular switch for ephrin cleavage in trans. Cell, 2005, 123(2): 291-304.
Janes P W, et al. Cytoplasmic relaxation of active Eph controls ephrin shedding by ADAM10, PLoS Biology, 2009, 7: e1000215.
Jones P T, et al. Replacing the complementarity-determining regions in a human antibody with those from a mouse. Nature, 1986, 321: 522-525.
Riechmann L, et al. Reshaping human antibodies for therapy. Nature, 1998, 332: 323-327.
Kabat E A, et al. Sequences of Proteins of Immunological Interest, 5th Ed. Public Health Service, National Institutes of Health, Bethesda, Md., 1991.
Karlin S & Altschul S F. Methods for assessing the statistical significance of molecular sequence features by using general scoring schemes. Proceedings of the National Academy of Science, 1190, 87: 2264-2268.
Karlin S & Altschul S F. Applications and statistics for multiple high-scoring segments in molecular sequences. Proceedings of the National Academy of Science. 1993, 90: 5873-5877.
Kobayashi H, et al. Tryptophan H33 plays an important role in pyrimidine (6-4) pyrimidone photoproduct binding by a high-affinity antibody. Protein Engineering, 1999, 12: 879-884.
Köhler G & Milstein C. Continuous cultures of fused cells secreting antibody of predefined specificity. Nature, 1975, 256: 495-497.
Kuby. Immunology, W. H. Freeman and Company: New York, 1992.
Lefranc M-P, et al. IMGT unique numbering for immunoglobulin and T cell receptor variable domains and Ig superfamily V-like domains. Developmental and Comparative Immunology. 2003, 27: 55-77.
Li J L, et al. DLL4-notch signaling mediates tumor resistance to anti-VEGF therapy in vivo. Cancer Research, 2011, 71: 6073-6083.
Lipper C H. et al. Structural basis for membrane-proximal proteolysis of substrates by ADAM10. Cell, 2023, 186: 3632-3641.
Long H, et al. Notch1 is a prognostic factor that is distinctly activated in the classical and proneural subtype of glioblastoma and that promotes glioma cell survival via the NF-κB(p65) pathway. Cell Death and Disease, 2018, 9: 158.
Luckow V A & Summers M D. Trends in the Development of Baculovirus Expression Vectors. Nature: Biotechnology, 1988, 6: 47-55.
Marks J D, et al. By-passing immunization: human antibodies from V-gene libraries displayed on phage. Journal of Molecular Biology, 1991, 222: 581-597.
Marks J D, et al. By-passing immunization: building high affinity human antibodies by chain shuffling. Bio/technology, 1992, 10: 779-783.
McAuliffe S M, et al. Targeting Notch, a key pathway for ovarian cancer stem cells, sensitizes tumors to platinum therapy. Proceedings of the National Academy of Science, 2012, 109: E2939-E2948.
McCafferty J, et al. Phage antibodies: filamentous phage displaying antibody variable domains. Nature, 1990, 348: 552-554.
Meng R D, et al. Gamma-Secretase inhibitors abrogate oxaliplatin-induced activation of the Notch-1 signaling pathway in colon cancer cells resulting in enhanced chemosensitivity. Cancer Research, 2009, 69: 573-582.
Morimoto K & Inouye K. Single-step purification of F(ab′)2 fragments of mouse monoclonal antibodies (immunoglobulins G1) by hydrophobic interaction high performance liquid chromatography using TSKgel Phenyl-5PW. Journal of Biochemical and Biophysical Methods, 1993, 24: 107-117.
Murphy G. The ADAMs: signalling scissors in the tumour microenvironment. Nature Reviews: Cancer, 2008, 8: 929-941.
Needleman S B & Wunsch C D. A general method applicable to the search for similarities in the amino acid sequence of two proteins. Journal of Molecular Biology, 1970, 48: 444-453.
O'Brien J, et al. Investigation of the Alamar Blue (resazurin) fluorescent dye for the assessment of mammalian cytotoxicity. European Journal of Biochemistry. 2000, 267(17): 5421-5426.
Oganesyan V, et al. Structural characterization of a human Fc fragment engineered for lack of effector functions. Acta Crystallographica. Section D. Biological Crystallography, 2008, 64: 700-704.
Pal S, et al. Targeting a chemorefractory COLO205 (BRAF V600E) cell line using substituted benzo[α]phenoxazines. RSC Advances, 2015, 5: 82549-82563.
Potts A H, et al. Ovarian cancer cell lines derived from non-serous carcinomas migrate and invade more aggressively than those derived from high-grade serous carcinomas. Scientific Reports, 2019, 9: 5515.
Presta L G, et al. Humanization of an antibody directed against IgE. Journal of Immunology, 1993, 151: 2623-2632.
Reddy P, et al. Functional analysis of the domain structure of tumor necrosis factor-alpha converting enzyme. Journal of Biological Chemistry, 2000, 275(19): 14608-14614.
Remington's Pharmaceutical Sciences, 20th ed., Mack Publishing Co., Easton. PA (2000)
Riechmann L, et al. Reshaping human antibodies for therapy. Nature, 1988, 332: 323-327.
Rios-Doria J. et al. A monoclonal antibody to ADAM17 inhibits tumor growth by inhibiting EGFR and non-EGFR-mediated pathways. Molecular Cancer Therapeutics, 2015, 14: 1637-1649.
Robbie G J, et al. A novel investigational Fc-modified humanized monoclonal antibody, motavizumab-YTE, has an extended half-life in healthy adults. Antimicrobial Agents and Chemotherapy, 2013, 57: 6147-6153.
Rothe C, et al. The human combinatorial antibody library HuCAL GOLD combines diversification of all six CDRs according to the natural immune system with a novel display method for efficient selection of high-affinity antibodies. Journal of Molecular Biology, 2008, 375: 1182-1200.
Rusch V. et al. Differential expression of the epidermal growth factor receptor and its ligands in primary nonsmall cell lung cancers and adjacent benign lung. Cancer Research, 1993, 53: 2379-2385.
Saha et al. Fully human monoclonal antibody targeting activated ADAM10 on colorectal cancer cells. Biomedicine & Pharmacotherapy, 2023, 161: 114494.
Seals D F & Courtneidge S A. The ADAMs family of metalloproteases: multidomain proteins with multiple functions. Genes & Development, 2003, 17: 7-30.
Seegar T C M, et al. Structural basis for regulated proteolysis by the α-Secretase ADAM10. Cell, 2017, 171(7): 1638-1648.
Sheets M D, et al. Efficient construction of a large nonimmune phage antibody library: the production of high-affinity human single-chain antibodies to protein antigens. Proceedings of the National Academy of Science, 1998, 95: 6157-6162.
Shields R L, et al. High resolution mapping of the binding site on human IgG1 for FcγRI, FcγRII, FcγRIII, and FcRn and design of IgG1 variants with improved binding to the FcγR. Journal of Biological Chemistry, 2001, 276: 6591-6604.
Sims M J. et al. A humanized CD18 antibody can block function without cell destruction. Journal of Immunology, 1993, 151: 2296-2308.
Smith D, et al. Optimization of cellular ELISA for assay of surface antigens on human synoviocytes. BioTechniques, 1997, 22: 952-957.
Smith K M. et al. The cysteine-rich domain regulates ADAM protease function in vivo. Journal of Cell Biology, 2002, 159(5): 893-902.
Smith T M, et al. Targeting ADAM10 in cancer and autoimmunity. Frontiers in Immunology. 2020, 11: 499.
Subik K, et al. The expression patterns of ER, PR, HER2, CK5/6, EGFRm Ki-67 and AR by immunohistochemical analysis in breast cancer cell lines. Breast Cancer: Basic and Clinical Research, 2010, 4: 35-41.
Timme C R, et al. Gamma-secretase inhibition attenuates oxaliplatin-induced apoptosis through increased Mcl-1 and/or Bcl-xL in human colon cancer cells. Apoptosis: An International Journal on Programmed Cell Death, 2013, 18: 1163-1174.
Vaughan T, et al. Human antibodies with sub-nanomolar affinities isolated from a large non-immunized phage display library. Nature: Biotechnology. 1996, 14: 309-314.
Verhoeyen M, et al. Reshaping human antibodies: grafting an antilysozyme activity. Science, 1988, 239: 1534-1536.
Wang Y Y, et al. ADAM 10 is associated with gastric cancer progression and prognosis of patients. Journal of Surgical Oncology, 2011, 103: 116-123.
White J M. ADAMs: modulators of cell-cell and cell-matrix interactions. Current Opinion in Cell Biology, 2003, 15(5): 598-606.
Wolfsberg T G & White J M. In Handbook of Proteolytic Enzymes, Second Edition. Barrett A J et al., eds., London: Academic Press, 2004: 709-14.
Wu Y, et al. Therapeutic antibody targeting of individual Notch receptors. Nature, 2010, 464: 1052-1057.
Xu K, et al. Host cell recognition by the henipaviruses: Crystal structures of the Nipah G attachment glycoprotein and its complex with ephrin-B3. Proceedings of the National Academy of Sciences, 2008, 105(29): 9953-9958.
Young P E, et al. Early detection of colorectal cancer recurrence in patients undergoing surgery with curative intent: current status and challenges. Journal of Cancer, 2014, 5(4): 262-271.

What is claimed is:

1. An ADAM10 binding molecule comprising:
(a) a heavy chain variable region comprising: a complementarity determining region (CDR)-1 domain comprising an amino acid sequence of SEQ ID NO. 1, a CDR-2 domain comprising an amino acid sequence of SEQ ID NO. 2, and a CDR-3 domain comprising an amino acid sequence of SEQ ID NO. 3, and
(b) a light chain variable region comprising: a CDR-1 domain comprising an amino acid sequence of SEQ ID NO. 4, a CDR-2 domain comprising an amino acid sequence of SEQ ID NO. 5, and a CDR-3 domain comprising an amino acid sequence of SEQ ID NO. 6.

2. The ADAM10 binding molecule of claim 1, wherein
(a) the heavy chain variable region comprises a CDR-1 domain consisting of an amino acid sequence of SEQ ID NO. 1, a CDR-2 domain consisting of an amino acid sequence of SEQ ID NO. 2, and a CDR-3 domain consisting of an amino acid sequence of SEQ ID NO. 3, and
(b) a light chain variable region comprising: a CDR-1 domain consisting of an amino acid sequence of SEQ ID NO. 4, a CDR-2 domain consisting of an amino acid sequence of SEQ ID NO. 5, and a CDR-3 domain consisting of an amino acid sequence of SEQ ID NO. 6.

3. The ADAM10 binding molecule of claim 1 or 2, wherein
(a) the heavy chain variable region comprises an amino acid sequence at least 95% identical to SEQ ID NO. 22, and
(b) the light chain variable region comprises an amino acid sequence at least 95% identical to SEQ ID NO. 23.

4. The ADAM10 binding molecule of any one of claims 1-3, wherein
(a) the heavy chain variable region comprises an amino acid sequence of SEQ ID NO. 22, and
(b) the light chain variable region comprises an amino acid sequence of SEQ ID NO. 23.

5. The ADAM10 binding molecule of any one of claims 1-4, wherein
(a) the heavy chain variable region consists of an amino acid sequence of SEQ ID NO. 22, and
(b) the light chain variable region consists of an amino acid sequence of SEQ ID NO. 23.

6. An ADAM10 binding molecule comprising:
(a) heavy chain complementarity determining region (CDR)-1, CDR-2, and CDR-3 domains contained within a heavy chain variable region comprising an amino acid sequence of SEQ ID NO. 22, and
(b) light chain CDR-1, CDR-2, and CDR-3 domains contained within a light chain variable region comprising an amino acid sequence of SEQ ID NO. 23.

7. The ADAM10 binding molecule of claim 6, wherein
(a) heavy chain CDR-1, CDR-2, and CDR-3 domains contained within a heavy chain variable region consisting of an amino acid sequence of SEQ ID NO. 22, and
(b) light chain CDR-1, CDR-2, and CDR-3 domains contained within a light chain variable region consisting of an amino acid sequence of SEQ ID NO. 23.

8. The ADAM 10 binding molecule of any one of claims 1-7, comprising
(a) a heavy chain comprising an amino acid sequence of SEQ ID NO. 29, and
(b) a light chain comprising an amino acid sequence of SEQ ID NO. 30.

9. The ADAM 10 binding molecule of any one of claims 1-8, comprising
(a) a heavy chain consisting of an amino acid sequence of SEQ ID NO. 29, and
(b) a light chain consisting of an amino acid sequence of SEQ ID NO. 30.

10. An ADAM10 binding molecule that specifically binds to the same epitope on human ADAM10 as an ADAM10 binding molecule according to any one of claims 1-9.

11. An ADAM10 binding molecule that competes with an ADAM10 binding molecule according to any one of claims 1-9 for binding to human ADAM10.

12. The ADAM10 binding molecule according to any one of claims 1-9, wherein the binding molecule is an antibody.

13. The ADAM10 binding molecule according to claim 12, wherein the antibody is a humanized antibody, a fully human antibody, a murine antibody, a chimeric antibody, a monoclonal antibody, a polyclonal antibody, a bi-specific antibody, or a multi-specific antibody.

14. The ADAM 10 binding molecule according to any one of claims 1-9, wherein the binding molecule is a Fv, a Fab, a F(ab)2, a Fab′, a dsFv fragment, a single chain Fv (scFV), an sc(Fv)2, a disulfide-linked (dsFv), a diabody, a triabody, a tetrabody, a minibody, or a single chain antibody.

15. The ADAM10 binding molecule according to any one of claims 1-9, comprising a heavy chain constant region.

16. The ADAM10 binding molecule according to claim 15, wherein the heavy-chain constant region is selected from the group consisting of alpha, delta, epsilon, gamma, and mu heavy chain constant regions.

17. The ADAM10 binding molecule according to claim 15, wherein the binding molecule is an IgA, IgD, IgE, IgG or IgM class immunoglobulin.

18. The ADAM10 binding molecule according to any one of claims 1-9, comprising a light chain constant region.

19. The ADAM10 binding molecule according to claim 18, wherein the light chain constant region is a lambda light chain constant region or a kappa light chain constant region.

20. A composition comprising an ADAM10 binding molecule according to any one of claims 1-19.

21. A pharmaceutical composition comprising an ADAM10 binding molecule according to any one of claims 1-19.

22. A host cell that produces an ADAM10 binding molecule according to any one of claims 1-19.

23. The host cell of claim 22, wherein the cell is a mammalian cell.

24. The host cell of claim 23, wherein the cell is a human cell.

25. The host cell of claim 23, wherein the cell is a murine cell.

26. An isolated nucleic acid molecule comprising a nucleotide sequence encoding an ADAM10 binding molecule according to of any one of claims 1-19.

27. A vector comprising a nucleic acid molecule according to claim 26.

28. A host cell comprising a nucleic acid molecule according to claim 26 or a vector according to claim 27.

29. The host cell of claim 28, wherein the cell is a mammalian cell.

30. The host cell of claim 29, wherein the cell is a human cell.

31. The host cell of claim 29, wherein the cell is a murine cell.

32. A method for inhibiting the proliferation of, or killing, tumor cells, the method comprising delivering to tumor cells an effective amount of an ADAM10 binding molecule according to any one of claims 1-19, or a composition according to claim 20, or a pharmaceutical composition according to claim 21.

33. The method of claim 32, wherein the tumor cells are selected from the group consisting of colorectal cancer cells, colon cancer cells, breast cancer cells, ovarian cancer cells, lung cancer cells, non-small cell lung cancer cells, brain cancer cells, glioma cells, glioblastoma cells, and neuroblastoma cells.

34. The method of claim 32 or 33, wherein the tumor cells overexpress, exhibit over-activity of, or are dependent on signaling of, Notch, epidermal growth factor receptor (EGFR), or erythropoietin-producing human hepatocellular (Eph) receptor.

35. The method of any one of claims 32-34, wherein the tumor cells are in vitro.

36. The method of any one of claims 32-34, wherein the tumor cells are in vivo.

37. A method of inhibiting a biological activity in cells or in a tissue, wherein the biological activity is selected from the group consisting of: (a) binding of ADAM10 to an ADAM10 substrate, (b) proteolytic cleavage of an ADAM10 substrate by ADAM10, (c) activation of an ADAM10 substrate, and (d), signaling by an ADAM10 substrate, wherein the method comprises delivering an effective amount of an ADAM10 binding molecule according to any one of claims 1-19, or a composition according to claim 20, or a pharmaceutical composition according to claim 21, to cells or a tissue that expresses or contains ADAM10, thereby inhibiting the biological activity in the cells or tissue.

38. The method of claim 37, wherein the cells or tissue are selected from the group consisting of colorectal cancer cells or tissue, colon cancer cells or tissue, breast cancer cells or tissue, ovarian cancer cells or tissue, lung cancer cells or tissue, non-small cell lung cancer cells or tissue, brain cancer cells or tissue, glioma cells or tissue, glioblastoma cells or tissue, and neuroblastoma cells or tissue.

39. The method of claim 37 or 38, wherein the cells or tissue are in vitro.

40. The method of any one of claims 37-39, wherein the cells or tissue are in vivo.

41. The method of any one of claims 37-40, wherein the ADAM10 substrate is a ligand of Notch, epidermal growth factor receptor (EGFR), or erythropoietin-producing human hepatocellular (Eph) receptor.

42. A method of treating cancer in a subject, the method comprising administering to a subject having cancer an effective amount of an ADAM10 binding molecule according to any one of claims 1-19, or a composition according to claim 20, or a pharmaceutical composition according to claim 21.

43. The method of claim 42, wherein the cancer is selected from the group consisting of colorectal cancer, colon cancer, breast cancer, ovarian cancer, lung cancer, non-small cell lung cancer, brain cancer, glioma, glioblastoma, and neuroblastoma.

44. The method of claim 42 or claim 43, wherein the cancer comprises tumor cells that overexpress, exhibit over-activity of, or are dependent on signaling of, Notch, epidermal growth factor receptor (EGFR), or erythropoietin-producing human hepatocellular (Eph) receptor.

45. The method of any one of claims 42-44, further comprising administering an additional active agent to the subject.

46. The method of claim 45, wherein the additional active agent is a chemotherapeutic agent.

47. The method of claim 45, wherein the additional active agent is an antibody, or antigen binding fragment thereof.

48. The method of claim 45, wherein the additional active agent is selected from the group consisting of afatinib, actinomycin, azacitidine, azathioprine, bevacizumab, bleomycin, bortezomib, carboplatin, capecitabine, cetuximab, cisplatin, chlorambucil, cyclophosphamide, cytarabine, daunorubicin, docetaxel, doxifluridine, doxorubicin, epirubicin, epothilone, erlotinib, etoposide, fluorouracil, gefitinib, gemcitabine, hydroxyurea, idarubicin, imatinib, irinotecan, mechlorethamine, mercaptopurine, methotrexate, mitoxantrone, olaparib, oxaliplatin, paclitaxel, panitumab, pazopanib, pemetrexed, poly(ADP-ribose) polymerase (PARP) inhibitors, tamoxifen, teniposide, tioguanine, topotecan, trastuzumab, tretinoin, valrubicin, vinblastine, vincristine, vindesine, vinorelbine, and vintafolide.

49. A method for detecting ADAM10 in a sample, the method comprising
(a) contacting a sample with an ADAM10 binding molecule according to any one of claims 1-19, or a composition according to claim 20, or a pharmaceutical composition according to claim 21; and
(b) detecting binding of the ADAM10 binding molecule to ADAM10, thereby detecting ADAM10 in the sample.

50. A method of determining whether a subject with a tumor is a candidate for treatment with an ADAM10 binding molecule, the method comprising:
(a) contacting a tumor sample from a subject, or cells therefrom, with an ADAM10 binding molecule according to any one of claims 1-19, or a composition according to claim 20, or a pharmaceutical composition according to claim 21; and
(b) performing an assay to determine whether the ADAM10 binding molecule binds to ADAM10 in the sample;
whereby if the ADAM10 binding molecule binds to ADAM0 in the sample the subject is a candidate for treatment with an ADAM10 binding molecule according to any one of claims 1-19, or a composition according to claim 20, or a pharmaceutical composition according to claim 21.

51. A method of determining whether a subject with a tumor is a candidate for treatment with an ADAM10 binding molecule, the method comprising:
(a) contacting a tumor sample from a subject, or cells therefrom, with an ADAM10 binding molecule according to any one of claims 1-19, or a composition according to claim 20, or a pharmaceutical composition according to claim 21; and
(b) performing an assay to determine whether the ADAM10 binding molecule inhibits proteolytic cleavage of an ADAM10 substrate in the sample:
whereby if the ADAM10 binding molecule inhibits proteolytic cleavage of the ADAM10 substrate in the sample the subject is a candidate for treatment with an ADAM10 binding molecule according to any one of claims 1-19, or a composition according to claim 20, or a pharmaceutical composition according to claim 21.

52. A method of determining whether a subject with tumor is a candidate for treatment with an ADAM10 binding molecule, the method comprising:
(a) contacting a tumor sample from a subject, or cells therefrom, with an ADAM10 binding molecule according to any one of claims 1-19, or a composition according to claim 20, or a pharmaceutical composition according to claim 21; and
(b) performing an assay to determine whether the ADAM10 binding molecule inhibits activation of or signaling of an ADAM10 substrate in the sample:
whereby if the ADAM10 binding molecule inhibits activation or signaling of the ADAM10 substrate in the sample the subject is a candidate for treatment with an ADAM10 binding molecule according to any one of claims 1-19, or a composition according to claim 20, or a pharmaceutical composition according to claim 21.

53. The method of any one of claims 50-52, further comprising administering an effective amount of an ADAM10 binding molecule according to any one of claims 1-19, or a composition according to claim 20, or a pharmaceutical composition according to claim 21, to the subject.

54. The method of any one of claims 50-53, wherein the tumor is selected from the group consisting of colorectal tumor, colon tumor, breast tumor, ovarian tumor, lung tumor, non-small cell lung tumor, brain tumor, glioma, glioblastoma, and neuroblastoma.

55. The method of claim 51 or 52, wherein the ADAM10 substrate is a ligand of Notch, epidermal growth factor receptor (EGFR), or erythropoietin-producing human hepatocellular (Eph) receptor.
